(19) United States
(12) Patent Application Publication
(10) Pub. No.: US 2024/0221942 A1
(43) Pub. Date: Jul. 4, 2024

(54) SYSTEMS AND METHODS FOR IDENTIFYING NOVEL AND DIVERGENT VIRUSES IN TRANSCRIPTOMES

(71) Applicant: The Wistar Institute of Anatomy and Biology, Philadelphia, PA (US)

(72) Inventors: Noam Auslander, Philadelphia, PA (US); Abdurrahman Elbasir, Philadelphia, PA (US)

(21) Appl. No.: 18/392,646

(22) Filed: Dec. 21, 2023

Related U.S. Application Data
(60) Provisional application No. 63/477,952, filed on Dec. 30, 2022.

(51) Int. Cl. G16H 50/20; G16B 30/10

(52) U.S. Cl. CPC G16H 50/20; G16B 30/10

(57) ABSTRACT

Systems and methods for identifying viral sequences in a subject of a species are provided. Sequence reads not associated with a reference genome of the species are obtained from a biological sample from the subject. At least a portion of each respective sequence read is encoded into a corresponding vector representing all or a portion of the sequence of the respective sequence read, thereby obtaining a plurality of vectors. Each sequence read is assigned a corresponding scalar model score by inputting a vector, in the plurality of vectors, corresponding to the sequence read into a model. Those sequence reads having a corresponding scalar model score that satisfies a first threshold score are selected as contig seeds. The plurality of sequence reads are aligned to these contig seeds through common k-mer sequences thereby forming a plurality of contigs which, in turn, are used to identify viral sequences in the subject.

### GOVERNMENT SUPPORT

This invention was made in part with government support under Project No. R00CA252025 awarded by the National Cancer Institute of the National Institutes of Health. The government has certain rights in the invention.

### INCORPORATION BY REFERENCE OF TABLES SUBMITTED AS TEXT FILES VIA EFS-WEB

The present Application contains Tables 1, 2, 3, 4, 5, 6, 7, 8, 9, and 10, which have each been submitted as a computer readable text file in ASCII format via EFS-Web and are hereby incorporated in their entirety by reference herein. The text files, which were created on Oct. 22, 2022, are named Table_1 (referred to in the present disclosure as “Table 1”), Table_2 (referred to in the present disclosure as “Table 2”), Table_3 (referred to in the present disclosure as “Table 3”), Table_4 (referred to in the present disclosure as “Table 4”), Table_5 (referred to in the present disclosure as “Table 5”), Table_6 (referred to in the present disclosure as “Table 6”), Table_7 (referred to in the present disclosure as “Table 7”), Table_8 (referred to in the present disclosure as “Table 8”), Table_9 (referred to in the present disclosure as “Table 9”), and Table_10 (referred to in the present disclosure as “Table 10”) are respectively 6 kilobytes, 1,389 kilobytes, 1,880 kilobytes, 4,396 kilobytes, 305 kilobytes, 26 kilobytes, 6 kilobytes, 4,332 kilobytes, 1,936 kilobytes, and 265 kilobytes in size.

### TECHNICAL FIELD

The present invention is directed to systems and methods for identifying novel and divergent viruses in transcriptomes.

### BACKGROUND

Viral infections have a causal role in approximately 15% of all cancer cases worldwide. See Morales-Sánchez et al., 2014, “Human viruses and cancer,” Viruses, (6), pg. 4047-4079, doi: 10.3390/v6104047. Viruses linked to cancer are generally divided into direct carcinogens, which drive an oncogenic transformation through viral oncogene expression, and indirect carcinogens, which may lead to cancer through mutagenesis associated with infection and inflammation. To date, seven viruses have been classified as direct carcinogenic agents in humans. See Krump et al., 2018, “Molecular mechanisms of viral oncogenesis in humans,” Nat Rev Microbiol (16), pg. 684-698. Among these, the high-risk subtypes of human papillomavirus (HPV) are the causative agent of approximately 5% of human cancers. Chronic hepatitis B virus (HBV) or hepatitis C virus (HCV) infections are associated with most hepatocellular carcinoma cases. More recently, advances in sequencing technologies have contributed to better appreciation of the high burden of viral infections in cancer, exemplified by the Kaposi's sarcoma herpesvirus and the Merkel cell polyomavirus, which were discovered based on nucleic acid subtraction to cause Kaposi's sarcoma and Merkel cell carcinoma, respectively. See Krump et al., 2018. The discovery of oncogenic viruses, starting with the Rous sarcoma virus, has been critical for understanding mechanisms driving cancer evolution and for improving cancer prevention and intervention strategies. However, the burden of viral infections in cancer is thought to remain underappreciated by much of the cancer research community. See Rous, 1911, “A Sarcoma of the fowl transmissible by an agent separable from the tumor cells,” J Exp Med (13), pg. 397-411; Moore et al., 2010, “Why do viruses cause cancer? Highlights of the first century of human tumour virology,” Nat Rev Cancer (10), pg. 878-889.

Since the advent of next-generation sequencing, new viral strains are typically identified from large-scale DNA or RNA sequencing data based on sequence similarity to known viruses. The Cancer Genome Atlas (TCGA) has become a principal resource for identification of viral sequences in cancer tissues. Several studies screened TCGA DNA sequencing data to characterize known viruses in cancers and analyze host integration sites for viruses such as HBV that integrate into the human genome. See Salyakina et al., 2013, “Viral expression associated with gastrointestinal adenocarcinomas in TCGA high-throughput sequencing data,” Hum Genomics (7), pg. 23, doi: 10.1186/1479-7364-7-23; Parfenov et al., 2014, “Characterization of HPV and host genome interactions in primary head and neck cancers,” Proc Natl Acad Sci USA (111), pg. 15544-15549. Other studies used RNA sequencing to screen for known viruses in the human transcriptome, and to discover novel viral isolates. See Cao et al., 2016, “Divergent viral presentation among human tumors and adjacent normal tissues,” Sci Rep (6), pg. 28294; Strong et al., 2013, “Differences in gastric carcinoma microenvironment stratify according to EBV infection intensity: implications for possible immune adjuvant therapy,” PLOS Pathog (9), pg. e1003341, doi: 10.1371/journal.ppat. 1003341; Khoury et al., 2013, “Landscape of DNA virus associations across human malignant cancers: analysis of 3,775 cases using RNA-Seq,” J Virol (87), pg. 8916-8926; Tang et al., 2013, “The landscape of viral expression and host gene fusion and adaptation in human cancer,” Nat Commun (4), pg. 2513, doi: 10.1038/ncomms3513. Most recently, a few studies combined DNA and RNA sequencing to quantify presence of known cancer-associated viruses in human cancers. See Cantalupo et al., 2018, “Viral sequences in human cancer,” Virology (513), pg. 208-216; Zapatka et al., 2020, “The landscape of viral associations in human cancers,” Nat Genet (52), pg. 320-330. However, the set of sequenced viral clades and the set of viral clades known to infect humans are both incomplete. Viruses and cancers have rapidly evolving genomes, and a new cancer-associated virus may have little sequence similarity to known viruses isolated outside of the tumor micro-environment. This issue is exacerbated when analyzing short reads, which are typical to RNA sequencing technologies. Therefore, discovery of new and divergent cancer viruses remains highly challenging with existing strategies. See Kellam, P., 1998, “Molecular identification of novel viruses,” Trends Microbiol (6), pg. 160-165. For detection of bacterial viruses from metagenomic DNA sequencing, several machine and deep learning techniques have been recently developed. These methods overcome some of the limitations associated with homology-based approaches and rapidly identify viral reads including novel and divergent viruses. See Ren et al., 2017, “VirFinder: a novel k-mer based tool for identifying viral sequences from assembled metagenomic data,” Microbiome (5), pg. 69; Ren et al., 2020, “Identifying viruses from metagenomic data using deep learning,” Quant Biol (8), pg. 64-77; Fang et al., 2019, “PPR-Meta: a tool for identifying phages and plasmids from metagenomic fragments using deep learning,” Gigascience (8), print doi: 10.1093/gigascience/giz066; Auslander et al., 2020, “Secker: alignment-free identification of bacteriophage genomes by deep learning,” Nucleic Acids Res (48), pg. e121, doi: 10.1093/nar/gkaa856. This suggests that deep learning methods to detect viral reads from RNA sequencing have a similar potential to uncover novel and divergent viruses in human tissues.

For instance, one conventional solution to characterize oncogenic processes operates by defining ‘mutation signatures’ that are associated with subsets of cancers and environmental factors, through the application of on non-negative matrix factorization (NMF). See Alexandrov et al., 2013, “Signatures of mutational processes in human cancer,” Nature 500(7463), pg. 415-421; Alexandrov et al., 2014, “Mutational signatures: the patterns of somatic mutations hidden in cancer genomes,” Current opinion in genetics & development, (24), pg. 52-60. Although this conventional technique has shown major success in characterizing mutational processes, NMF only facilitates linear modeling and may not capture complex mutational patterns.

Furthermore, viral infections have a causal role in approximately 15% of all cancer cases worldwide. See Morales-Sánchez et al., 2014, “Human viruses and cancer,” Viruses, 6(10), pg. 4047-4079. Viruses linked to cancer are generally divided into direct carcinogens, which drive an oncogenic transformation through viral oncogene expression, and indirect carcinogens, which may lead to cancer through mutagenesis associated with infection and inflammation. Conventionally, seven viruses have been classified as direct carcinogenic agents in humans. See Krump et al., 2018, “Molecular mechanisms of viral oncogenesis in humans,” Nat Rev Microbiol, 16(11), pg. 684-698. Among these, the high-risk subtypes of human papillomavirus (HPV) are the causative agent of approximately 5% of human cancers. Chronic hepatitis B virus (HBV) or hepatitis C virus (HCV) infections are associated with most hepatocellular carcinoma cases. More recently, advances in sequencing technologies have contributed to better appreciation of the high burden of viral infections in cancer, demonstrated by the Kaposi's sarcoma herpesvirus and the Merkel cell polyomavirus, which were discovered based on nucleic acid subtraction to cause Kaposi's sarcoma and Merkel cell carcinoma, respectively. See Krump et al., 2018. The discovery of oncogenic viruses, starting with the Rous sarcoma virus, has been critical for understanding mechanisms driving cancer evolution and for improving cancer prevention and intervention strategies. See Rous, 1911, “A Sarcoma of the Fowl Transmissible by an Agent Separable from the Tumor Cells,” J Exp Med, 13(4), pg. 397-411. However, the burden of viral infections in cancer is thought to be underappreciated by much of the cancer community. See Moore et al., 2010, “Why do viruses cause cancer? Highlights of the first century of human tumour virology,” Nat Rev Cancer, 10(12), pg. 878-889.

Given the above-background, what is needed in the art are improved systems and methods for identifying novel and divergent viruses in cancer transcriptomes.

### SUMMARY

The present disclosure addresses the shortcomings disclosed above by providing systems and methods for identifying novel and divergent viruses in cancer transcriptomes.

About 15% of human cancer cases are attributed to viral infections. Yet, virus expression in tumor tissues has been mostly studied by aligning tumor RNA sequencing reads to databases of known viruses. To allow identification of divergent viruses and rapid characterization of the tumor virome, the present disclosure provides systems and methods, also known as “viRNAtrap,” that provides an alignment-free pipeline to identify viral reads and assemble viral contigs. The present disclosure applies viRNAtrap, which is based on a deep learning model trained to discriminate viral RNAseq reads, to 14 cancer types from The Cancer Genome Atlas (TCGA). The present disclosure determined that expression of exogenous cancer viruses is associated with better overall survival. In contrast, expression of human endogenous viruses is associated with worse overall survival. Using viRNAtrap, the present disclosure uncovers expression of unexpected and divergent viruses that have not previously been implicated in cancer. The viRNAtrap pipeline provides a way forward to study viral infections associated with different clinical conditions.

More particularly, in some embodiments, the present disclosure provides a framework, also known as “viRNAtrap,” that employs model to accurately distinguish viral reads from RNA sequencing, and utilizes the model scores to assemble viral contigs. One embodiment of the present disclosure applies viRNAtrap to multiple cancer types from TCGA (selected based on potential viral relevance to oncogenesis), to characterize the landscape of viral infections in the human cancer transcriptome. The present disclosure provides an ability to identify different types of viruses that are expressed in tumors by constructing three viral databases and comparing findings to sequences in those databases.

In some embodiments, the present disclosure first evaluates known cancer-associated viruses that are expressed in different tumor types. A database of potentially functional human endogenous retroviruses (HERVs) is curated and expression patterns of different HERVs are analyzed across human cancers to find that HERV expression associated with poor survival rates. Finally, the present disclosure employs a model to identify divergent viruses that are expressed in tumor tissues. Notably, application of these disclosed techniques identifies Redondoviridae members that are expressed in head and neck carcinomas, a Siphoviridae member that is expressed in 10% high grade serious ovarian cancers, and a Betairdovirinae member that is expressed in more than 25% of endometrial cancer samples. Accordingly, the present disclosure provides a learning-based method to identify viruses from human RNA sequencing and demonstrates its ability to rapidly characterize viruses that are expressed in tumors and uncover viral instances that have not been previously found in these samples using alignment-based methods.

In some embodiments, the systems and methods of the present disclosure are applied to identify new viruses that are expressed in a variety of other malignancies, introducing new avenues to study viral diseases.

Accordingly, in some embodiments, the present disclosure is directed to providing systems and methods for modeling cancer evolution for prediction with neural networks.

In some embodiments, the systems and methods of the present disclosure are configured to characterize the interplay between driver mutations and aneuploidy in tumor evolution and identify determinants of clinical outcome and therapeutic vulnerabilities.

In some embodiments, the systems and methods of the present disclosure are configured to distinguish interactions between driver mutations and aneuploidy across cancer types and introduce tumor classification based on the landscape of driver-aneuploidy interactions.

In some embodiments, the systems and methods of the present disclosure are configured to derive the mutational signatures of DNA damage response (DDR) components that predict accumulation of oncogenic mutations and aneuploidy.

In some embodiments, the systems and methods of the present disclosure are configured to identify differences in therapeutic vulnerabilities and clinical outcomes based on the DDR patterns and the inferred tumor type classification.

In some embodiments, the systems and methods of the present disclosure are configured to introduce computational approaches to represent snapshot genomic data through temporal and functional ordering of genetic events to model tumor progression.

In some embodiments, the systems and methods of the present disclosure are configured to develop computational approaches to deconvolute the temporal order of genetic events observed in tumors from snapshot genomic data.

In some embodiments, the systems and methods of the present disclosure are configured to developed computational approaches to identify events and tumors types that follow linear versus clonal evolution with respect to mutations, copy number alterations and chromosomal aberrations.

In some embodiments, the systems and methods of the present disclosure are configured to classify genetic events with distinct contribution to tumor progression and introduce ordering of different classes of oncogenic events.

In some embodiments, the systems and methods of the present disclosure are configured to develop neural network frameworks to learn dynamics in tumor evolution from different data types and predict phenotypic features and clinical outcome.

In some embodiments, the systems and methods of the present disclosure are configured to develop a neural network technique to identify combinations of mutations that predict clinical outcomes.

In some embodiments, the systems and methods of the present disclosure are configured to develop an approach to identify complex combinations of genetic and epigenetic events that predict clinical outcomes.

In some embodiments, the systems and methods of the present disclosure are configured to identify and characterize unique viruses and bacteria that are associated with cancer.

Turning to more specific embodiments, an aspect of the present disclosure provides a method for identifying a viral sequence in a subject of a species. The method includes using a computer system. The computer system includes one or more processing cores and a memory. The method includes obtaining, in electronic form, a plurality of nucleic acid sequence reads from a biological sample obtained from the subject. The plurality of sequence reads is free of sequence reads associated with a reference genome of the species. Moreover, the method includes encoding at least a portion of each respective sequence read into a corresponding vector that represents a sequence of the respective sequence read, thereby obtaining a plurality of vectors. Each respective sequence read in the plurality of sequence reads is assigned a corresponding scalar model score by inputting the vector, in the plurality of vectors, corresponding to the respective sequence read, into a model. Additionally, a subset of the plurality of sequence reads is selected as a plurality of contig seeds. Each sequence read in the subset of sequence reads has a corresponding scalar model score that satisfies a first threshold score. Sequence reads in the plurality of sequence reads are aligned to the plurality of contig seeds through common k-mer sequences. The plurality of contigs is used to identify one or more viral sequences in the subject of the species.

In some such embodiments, the species is human.

In some such embodiments, each sequence read has a length of between 30 base pairs and 400 base pairs. In some such embodiments, the plurality of sequence reads is transcriptomic sequence reads. In some such embodiments, the plurality of sequence reads is genomic sequence reads. In some such embodiments, the plurality of sequence reads includes 10,000 or more sequence reads that each have a length of 35 nucleic acids or more. In some such embodiments, the plurality of sequence reads includes 100,000 or more sequence reads that each have a length of 35 nucleic acids or more.

In some such embodiments, the encoding is one hot encoding.

In some such embodiments, the model includes a one-dimensional convolutional layer followed by one or more fully connected layers. In some such embodiments, the model assigns the corresponding model score using an activation function in the final fully connected layer in the one or more fully connected layers. In some such embodiments, the activation function is a sigmoid activation function. In some such embodiments, the one or more fully connected layers is three or more fully connected layers.

In some such embodiments, each corresponding scalar model score is a value between zero and one and a sequence read in the plurality of sequence reads satisfies the first threshold score when it has a value that is between 0.60 and 1.0, or a value that is between 0.70 and 1.0.

In some such embodiments, the model includes 1,000 or more weights, or 10,000 or more weights, that are evaluated by the model during the assigning for each respective sequence read in the plurality of sequence reads.

In some such embodiments, the aligning to a respective contig seed in the plurality of contig seeds terminates when an average scalar model score for sequence reads aligning to the respective contig seed fails to satisfy a second threshold score.

In some such embodiments, each corresponding scalar model score is a value between zero and one and the average scalar model score for sequence reads aligning to the respective seed fails to satisfy the second threshold score when the average scalar model score is less than 0.60, or less than 0.50.

In some such embodiments, the common k-mer sequences have a length of 24 base pairs.

In some such embodiments, the common k-mer sequences have a common base pair length, in which the common base pair length is an integer between 12 and 45.

Another aspect of the present disclosure a computer system for identifying a viral sequence in a subject of a species. The computer system includes at least one processor and a memory. The memory stores at least one program for execution by the at least one processor. The at least one program includes instructions for obtaining, in electronic form, a plurality of nucleic acid sequence reads from a biological sample obtained from the subject, in which the plurality of sequence reads is free of sequence reads associated with a reference genome of the species. The at least one program further includes instructions for encoding at least a portion of each respective sequence read into a corresponding vector that represents a sequence of the respective sequence read, which obtains a plurality of vectors. The at least one program further includes instructions for assigning each respective sequence in the plurality of sequence reads a corresponding scalar model score by inputting the vector, in the plurality of vectors, corresponding to the respective sequence read into a model. Moreover, the at least one program further includes instructions for selecting a subset of the plurality of sequence reads as a plurality of contig seeds. Each sequence read in the subset of sequence reads has a corresponding scalar model score that satisfies a first threshold score. Additionally, the at least one program further includes instructions for aligning sequence reads in the plurality of sequence reads to the plurality of contig seeds through common k-mer sequences thereby forming a plurality of contigs. Moreover, the at least one program further includes instructions for using the plurality of contigs to identify one or more viral sequences in the subject of the species.

Yet another aspect of the present disclosure is directed to providing a non-transitory computer-readable storage medium having stored thereon program code instructions. When executed by a processor, the program code instructions cause the processor to perform a method for identifying a viral sequence in a subject of a species. The method includes obtaining, in electronic form, a plurality of nucleic acid sequence reads from a biological sample obtained from the subject. The plurality of sequence reads is free of sequence reads associated with a reference genome of the species. Moreover, the method includes encoding at least a portion of each respective sequence read into a corresponding vector that represents a sequence of the respective sequence read, which obtains a plurality of vectors. Furthermore, the method includes assigning each respective sequence read in the plurality of sequence reads a corresponding scalar model score by inputting the vector, in the plurality of vectors, corresponding to the respective sequence read into a model. Additionally, the method includes selecting a subset of the plurality of sequence reads as a plurality of contig seeds. Each sequence read in the subset of sequence reads has a corresponding scalar model score that satisfies a first threshold score. The method further includes aligning sequence reads in the plurality of sequence reads to the plurality of contig seeds through common k-mer sequences, which forms a plurality of contigs. Further, the method includes using the plurality of contigs to identify one or more viral sequences in the subject of the species.

Yet another aspect of the present disclosure is a method for determining a prognosis of a subject surviving a cancer.

The method includes using a computer system. The computer system includes one or more processing cores and a memory.

The method further includes obtaining, in electronic form, a plurality of nucleic acid sequence reads from a biological sample obtained from the subject. The plurality of sequence reads is free of sequence reads associated with a reference genome of the species of the subject.

Furthermore, the method includes determining whether sequence reads from an exogenous virus are present in the plurality of sequence reads. When sequence reads from an exogenous virus are present in the plurality of sequence reads, the method further includes up-weighting the prognosis that the subject will survive the cancer. When the plurality of sequence reads is free of sequences from an exogenous virus, the method further includes down-weighting the prognosis that the subject will survive the cancer.

In some such embodiments, the cancer is endometrial cancer.

In some such embodiments, the biological sample is a tumor biopsy.

In some such embodiments, the exogenous virus is an arthropod virus.

In some such embodiments, the arthropod virus is in the Betairidovirinae family.

In some such embodiments, the arthropod virus is Armadillidium vulgare iridescent virus.

Yet another aspect of the present disclosure is directed to providing a method for determining a prognosis of a subject surviving a cancer. The method includes using a computer system. The computer system includes one or more processing cores and a memory. The method includes obtaining, in electronic form, a plurality of nucleic acid sequence reads from a biological sample obtained from the subject, in which the plurality of sequence reads is free of sequence reads associated with a reference genome of the species of the subject. Moreover, the method includes determining whether sequence reads encoding YP_009046765, YP_009046752 and YP_009046774 are present in the plurality of sequence reads. When the plurality of sequence reads indicate that YP_009046765, YP_009046752 or YP_009046774 is highly expressed in the biological sample, the method further includes down-weighting the prognosis of the subject surviving the cancer. When the plurality of sequence reads indicate that YP_009046765, YP_009046752 or YP_009046774 is not highly expressed in the biological sample, the method includes up-weighting the prognosis of the subject surviving the cancer.

In some such embodiments, the cancer is endometrial cancer.

The systems, methods, devices, and non-transitory computer readable storage medium of the present invention have other features and advantages that will be apparent from, or are set forth in more detail in, the accompanying drawings, which are incorporated herein, and the following Detailed Description, which together serve to explain certain principles of exemplary embodiments of the present invention.

### BRIEF DESCRIPTION OF THE DRAWINGS

FIG. 1 illustrates an exemplary system topology including a computer system, in accordance with an exemplary embodiment of the present disclosure.

FIGS. 2A, 2B, 2C, and 2D collectively provide a flow chart illustrating exemplary methods for identifying a viral sequence in a subject of a species, in which dashed boxes indicate optional features, in accordance with some embodiments of the present disclosure;

FIG. 3 provides another flow chart illustrating exemplary methods for determining a prognosis of a subject surviving cancer, in which dashed boxes indicate optional features, in accordance with some embodiments of the present disclosure;

FIG. 4 provides yet another flow chart illustrating exemplary methods for determining a prognosis of a subject surviving cancer, in which dashed boxes indicate optional features, in accordance with some embodiments of the present disclosure;

FIGS. 5A, 5B, 5C, and 5D collectively illustrate training and evaluation of a viRNAtrap framework of the present disclosure, in which: FIG. 5A illustrates a schematic overview of the viRNAtrap framework, in which unmapped reads are extracted and given as input to the neural network, to extract the viral reads and assemble viral contigs, that were compared against three viral databases using blastn; FIG. 5B illustrate receiver-operating characteristic and precision-recall curves showing the model performance when viRNAtrap was applied to the test set; FIG. 5C illustrates bar plots showing different metrics to evaluate the model performance for the test set; and FIG. 5D illustrates a phylogenetic tree showing the model scores for sequences from different human viruses with the respective virus classification (using average assigned score for each virus).

FIGS. 6A, 6B, and 6C collectively illustrate reference human viruses expressed in different tumor types in accordance with embodiments of the present disclosure, in which: FIG. 6A illustrates a heatmap showing the total number of virus-positive samples identified from RNA-sequencing in different tumor tissues, top panels show the fraction of tumor and non-cancer samples in which viruses were identified, right panels show the number of viruses found in tumor and non-cancer samples; FIG. 6B illustrates violin plots comparing the tumor mutation burden (TMB) and the number of chromosome-level copy number alteration (CNA) between cancer patients where expression of specific viruses, the high-risk alpha papilloma or hepatitis B viruses, was detected vs those patients where expression of those viruses was not detected, in which black dots represent the medians, and the boundaries of the violin plots refer to the maximum and minimum values, respectively; and FIG. 6C illustrates Kaplan-Meier curves comparing the survival rates between patients where viral reads were detected (blue curves) vs those where viral reads were not detected (red curves), in which the log rank and proportional hazards (PH) p-values are reported.

FIGS. 7A, 7B. 7C, collectively illustrate human endogenous retroviruses (HERVs) expressed in different cancer types in accordance with embodiments of the present disclosure, in which: FIG. 7A illustrates a heatmap clustogram clustering the proportion of HERVs across different tumor types, where the rows are 14 TCGA tumor types, the 36 columns are the 36 distinct HERVs with the highest expression in human cancers, mapped to unique regions in the genome (Table 5); FIG. 7B illustrates Kaplan-Meier curves comparing the survival rates between patients in which any HERV reads were detected (blue curves) versus those in which no HERV reads were detected (red curves). The log rank and proportional hazards (PH) p-values are reported; and FIGS. 7C and 7D collectively illustrates heatmaps showing somatic mutations in major cancer driver genes (selected as the most frequency mutated driver genes in these samples, upper panel) and the expression of HERVs that are significantly associated with survival in colorectal and endometrial cancers of FIG. 7C and in renal and hepatocellular cancers of FIG. 7D.

FIGS. 8A, 8B, 8C, 8D, 8E, 8F, and 8G collectively illustrate unexpected and divergent viruses infecting different host taxa across TCGA samples in accordance with embodiments of the present disclosure, in which FIG. 8A illustrates unexpected and divergent viruses expressed in TCGA samples, where each row in the matrix represents one virus and the entry in each column indicates the number of cancer samples of each type in which each virus was detected, the canonical hosts of each virus are depicted at the left of the matrix, and to right, the aggregate number of tumor and normal samples including reads of each virus are shown in a bar plot; FIG. 8B illustrates Kaplan-Meier curves comparing the survival rates between patients in which IIV31 reads were detected (blue curves) vs those where viral reads were not detected (red curves), in which The log rank and proportional hazards (PH) p-values are reported; FIGS. 8C and 8D collectively illustrate box plots comparing the chromosome-level copy number alteration (FIG. 8C) and the tumor mutation burden (FIG. 8D) between cancer patients where IIV31 is found (blue) and patients where IIV31 is not found (red); FIG. 8E illustrates box plots comparing CIBERSORT-inferred proportions of regulatory T cells (Tregs) and CD8 T cells between patients positive and negative for IIV31; FIG. 8E illustrates Trichomonas vaginalis and mutations in PTEN, CTNNB1 and PIK3R1 are significantly associated with IIV31 presence, in which Fisher's exact test p-values are provided; and FIG. 8F illustrates a bar plot comparing the fold change (relative to GAPDH) between the COV318 cell line that was predicted as Geobacillus-positive, and the OVISE cell line that was used as control, in which the t-test p-value is provided.

FIG. 9 illustrates a chart that depicts proportions of TCGA samples that are identified as virus-positive by viRNAtrap that were also verified as virus-positive through TCGA clinical information in accordance with embodiments of the present disclosure, in which, from left to right: HR-HPV-positive in CESC, HR-HPV-positive in HNSC, HBV-positive in LIHC and HCV-positive in LIHC HR-HPV: high-risk human papilloma virus; HBV: hepatitis B virus; HCV: hepatitis C virus.

FIGS. 10A and 10B collectively illustrate charts in accordance with embodiments of the present disclosure depicting clinical and genomic correlates of Armadillidium vulgare iridescent virus (IIV31) expression in endometrial cancers, in which: FIG. 10A illustrates heatmaps showing IIV31 proteins expressed in different tumors, microsatellite instability, chromosomal aneuploidy, and tumor mutation burden (TMB) across endometrial cancer samples; and FIG. 10B illustrates Kaplan-Meier survival curves comparing survival based on presence (blue) or absence (red) of different IIV31 proteins in endometrial cancer samples.

It should be understood that the appended drawings are not necessarily to scale, presenting a somewhat simplified representation of various features illustrative of the basic principles of the invention.

### DETAILED DESCRIPTION

Identification of viruses from tumor RNA sequencing allows for the potential discovery of new carcinogenic agents and mechanisms. Discovery of novel and divergent viral species that contribute to cancer initiation and progression is crucial for development of new therapeutics, including vaccinations, screening practices, and antimicrobial treatments. Viruses are currently identified from sequencing reads based on similarity to known viruses. Additional details and information is found at Kostic, et al., 2011, “PathSeq: software to identify or discover microbes by deep sequencing of human tissue,” Nat Biotechnol (29), pg. 393-396, doi: 10.1038/nbt. 1868, which is hereby incorporated by reference in its entirety for all purposes. However, when studying viruses from short reads, typical with Illumina-based RNA sequencing, reads originating from divergent viruses may share little sequence similarity to known viruses, rendering the identification of novel viruses highly challenging.

To address this challenge, the present disclosure provides alignment-free models to identify viral reads from RNAseq and assemble viral contigs. These contigs can be aligned to different viral databases, as demonstrated by the present disclosure, to rapidly identify viral expression of interest in tumor samples. In one aspect, the present disclosure curates a database of HERVs that includes intact retroviral genes in the human genome and survey the expression of these viruses across different cancer tissues. Through a database of divergent viruses, the present disclosure demonstrates that the disclosed models identify viruses in TCGA samples that were not detected in previous studies. This is enabled through integrative systems and methods of the present disclosure that use model scores to assemble viral reads rather than aligning short divergent reads to viral databases or applying de-novo assembly to many unmapped reads. Importantly, in some embodiments, the output of the disclosed models are alternatively used as input to motif search tools, to potentially identify highly divergent viruses. Because the disclosed models are trained to distinguish viral from human sequences, model predictions for sequences derived from a range of other organisms is not defined. In some embodiments, the systems and methods of the present disclosure train models to identify viruses from a variety of other organisms, and achieve higher sensitivity for viral detection.

In one aspect, the present disclosure uses the disclosed models to characterize viruses that are expressed across multiple cancer tissues from TCGA and analyzes their genomic and survival correlates. Interestingly, using the disclosed models, the present disclosure determines that while the expression of exogenous cancer viruses is associated with improved survival, the expression of human endogenous viruses is strictly associated with poor survival rates. Expression of a virus of the subfamily Betairidovirinae, which are pathogens of insects, found in endometrial cancer tissues is similarly associated with significantly better overall patient survival. For all divergent viruses reported in the present disclosure, the presence and classification of multiple viral reads is verified by targeted blastn- and blastx-based sequence analyses in different samples.

One interesting divergent virus the models of the present disclosure found is IIV31 from the subfamily Betairidoovirinae, which was frequently detected in UCEC TCGA samples. Interestingly, IIV6, a very close relative of IIV31, can infect a variety of vertebrates including mice, and induces an immune response in mammalian tissues. Additional details and information is found at Newman et al., 2015, “Robust enumeration of cell subsets from tissue expression profiles,” Nat Methods (12), pg. 453-457; Ahlers et al., 2016, “Invertebrate Iridescent Virus 6, a DNA Virus, Stimulates a Mammalian Innate Immune Response through RIG-I-Like Receptors,” PLOS One (11), pg. e0166088, doi: 10.1371/journal.pone.0166088, each of which is hereby incorporated by reference in its entirety for all purposes. Thus, one possibility is that IIV31 is transmitted to the uterus through another insect, such as the crab louse. While the present disclosure has not yet confirmed the source of this virus, the present disclosure results imply that its presence may be a direct or indirect consequence of Trichomonas vaginalis infection. Therefore, the present disclosure illustrates that the disclosed models are sufficiently powerful to identify a previously unknown viral transcript in tumor samples, whether oncogenic or neutral. Through this analysis, the present disclosure also identified TV reads in multiple endometrial cancer samples, indicating a possible new association between TV and endometrial cancer, similar to the known association of TV with cervical cancer. Additional details and information is found at Yang et al., 2018, “Trichomonas vaginalis infection-associated risk of cervical cancer: A meta-analysis,” Eur J Obstet Gynecol Reprod Biol (228), pg. 166-173, which is hereby incorporated by reference in its entirety for all purposes. One of the established pathogenic mechanisms of TV infection in humans, which may also explain the frequent HPV coinfection, is that TV secretes exosomes that have the effect of suppressing CXCL8. Additional details and information is found at Twu et al., 2013, “Trichomonas vaginalis exosomes deliver cargo to host cells and mediate host:parasite interactions,” PLOS Pathog (9), pg. e1003482, doi: 10.1371/journal.ppat.1003482, which is hereby incorporated by reference in its entirety for all purposes. Interestingly, low expression of CXCL8, like infection with TV, has been associated with favorable prognosis in cervical cancer. Additional details and information is found at Wu et al., 2019, “Identification of Key Genes and Pathways in Cervical Cancer by Bioinformatics Analysis,” Int J Med Sci pg. (16), pg. 800-812, which is hereby incorporated by reference in its entirety for all purposes. Thus, it is possible that the presence of IIV31 is a secondary infection in patients already infected with TV or some other pathogen that suppresses the human anti-viral response.

Importantly, the present disclosure identified E2 Geobacillus virus in 10% of high-grade, serous ovarian cancers, making it the most frequently expressed virus in this cancer type. The present disclosure experimentally verified that E2 Geobacillus is indeed expressed in cell lines. The present disclosure also found expression of a Redondoviridae member in head and neck cancers that was not previously reported. Additional details and information is found at Taylor, et al., 2021, “Redondovirus Diversity and Evolution on Global, Individual, and Molecular Scales,” J Virol (95), pg. e0081721, doi: 10.1128/jvi.00817-21, which is hereby incorporated by reference in its entirety for all purposes. The present disclosure calls for a study of the role of Redondoviridae in tumor initiation and progression, as this family of viruses was only recently detected in humans and associated with different clinical conditions.

Accordingly, the present disclosure provides models for alignment free identification of viruses from data such as RNAseq, allowing rapid characterization of viral expression and detection of divergent viruses. The systems and methods of the present disclosure are applicable to tumor tissues from TCGA, in order to uncover expression patterns of different groups of viruses. The present disclosure provides previously unrecognized associations between several forms of cancer and several unexpected viral clades, including viral clades canonically found in produce and in insect parasites of humans. In some embodiments, the present disclosure employs the disclosed models to find viruses that contribute to other malignancies.

Reference will now be made in detail to embodiments, examples of which are illustrated in the accompanying drawings. In the following detailed description, numerous specific details are set forth in order to provide a thorough understanding of the present disclosure. However, it will be apparent to one of ordinary skill in the art that the present disclosure may be practiced without these specific details. In other instances, well-known methods, procedures, and components have not been described in detail so as not to unnecessarily obscure aspects of the embodiments.

It will also be understood that, although the terms first, second, etc. may be used herein to describe various elements, these elements should not be limited by these terms. These terms are only used to distinguish one element from another. For instance, a first subject could be termed a second subject, and, similarly, a second subject could be termed a first subject, without departing from the scope of the present disclosure. The first subject and the second subject are both subjects, but they are not the same subject.

The terminology used in the present disclosure is for the purpose of describing particular embodiments only and is not intended to be limiting of the invention. As used in the description of the invention and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

The foregoing description included example systems, methods, techniques, instruction sequences, and computing machine program products that embody illustrative implementations. For purposes of explanation, numerous specific details are set forth in order to provide an understanding of various implementations of the inventive subject matter. It will be evident, however, to those skilled in the art that implementations of the inventive subject matter may be practiced without these specific details. In general, well-known instruction instances, protocols, structures, and techniques have not been shown in detail.

The foregoing description, for purpose of explanation, has been described with reference to specific implementations. However, the illustrative discussions below are not intended to be exhaustive or to limit the implementations to the precise forms disclosed. Many modifications and variations are possible in view of the above teachings. The implementations are chosen and described in order to best explain the principles and their practical applications, to thereby enable others skilled in the art to best utilize the implementations and various implementations with various modifications as are suited to the particular use contemplated.

In the interest of clarity, not all of the routine features of the implementations described herein are shown and described. It will be appreciated that, in the development of any such actual implementation, numerous implementation-specific decisions are made in order to achieve the designer's specific goals, such as compliance with use case- and business-related constraints, and that these specific goals will vary from one implementation to another and from one designer to another. Moreover, it will be appreciated that such a design effort might be complex and time-consuming, but nevertheless be a routine undertaking of engineering for those of ordering skill in the art having the benefit of the present disclosure.

### Definitions

About or Approximately. As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. Where particular values are described in the application and claims, unless otherwise stated, the term “about” means within an acceptable error range for the particular value. The term “about” can have the meaning as commonly understood by one of ordinary skill in the art. The term “about” can refer to ±10%. The term “about” can refer to ±5%.

If. As used herein, the term “if” may be construed to mean “when” or “upon” or “in response to determining” or “in response to detecting,” depending on the context. Similarly, the phrase “if it is determined” or “if [a stated condition or event] is detected” may be construed to mean “upon determining” or “in response to determining” or “upon detecting [the stated condition or event]” or “in response to detecting [the stated condition or event],” depending on the context.

Microorganism. As used herein, the term “microorganism,” or “microbe,” refers to a microscopic organism. In some embodiments, the term “microorganism” will be understood to include bacteria, fungi, protozoa (e.g., protozoan parasites), viruses (e.g., DNA viruses and/or RNA viruses), algae, archaea, phages, and/or helminths (e.g., multicellular eukaryotic parasites). In some embodiments, a microorganism is a single-celled organism and/or a colony of single-celled organisms. In some embodiments, a microorganism is eukaryotic or prokaryotic. In some embodiments, a microorganism is a pathogen (e.g., disease-causing), such as a human, animal, or plant-infective pathogen.

Examples of bacteria include, but are not limited to, disease-causing agents such as Acinetobacter baumanii, Actinobacillus sp., Actinomycetes, Actinomyces sp. (such as Actinomyces israelii and Actinomyces naeslundii), Aeromonas sp. (such as Aeromonas hydrophila, Aeromonas veronii biovar sobria (Aeromonas sobria), and Aeromonas caviae), Anaplasma phagocytophilum, Anaplasma marginale Alcaligenes xylosoxidans, Acinetobacter baumanii, Actinobacillus actinomycetemcomitans, Bacillus sp. (such as Bacillus anthracis, Bacillus cereus, Bacillus subtilis, Bacillus thuringiensis, and Bacillus stearothermophilus), Bacteroides sp. (such as Bacteroides fragilis), Bartonella sp. (such as Bartonella bacilliformis and Bartonella henselae), Bifidobacterium sp., Bordetella sp. (such as Bordetella pertussis, Bordetella parapertussis, and Bordetella bronchiseptica), Borrelia sp. (such as Borrelia recurrentis, and Borrelia burgdorferi), Brucella sp. (such as Brucella abortus, Brucella canis, Brucella melintensis and Brucella suis), Burkholderia sp. (such as Burkholderia pseudomallei and Burkholderia cepacia), Campylobacter sp. (such as Campylobacter jejuni, Campylobacter coli, Campylobacter lari and Campylobacter fetus), Capnocytophaga sp., Cardiobacterium hominis, Chlamydia trachomatis, Chlamydophila pneumoniae, Chlamydophilapsittaci, Citrobacter sp. (such as Coxiella burnetii, Corynebacterium sp. (such as, Corynebacterium diphtheriae, Corynebacterium jeikeum and Corynebacterium), Clostridium sp. (such as Clostridium perfringens, Clostridium dificile, Clostridium botulinum and Clostridium tetani), Eikenella corrodens, Enterobacter sp. (such as Enterobacter aerogenes, Enterobacter agglomerans, Enterobacter cloacae and Escherichia coli, including opportunistic Escherichia coli, such as enterotoxigenic E. coli, enteroinvasive E. coli, enteropathogenic E. coli, enterohemorrhagic E. coli, enteroaggregative E. coli and uropathogenic E. coli), Enterococcus sp. (such as Enterococcus faecalis and Enterococcus faecium), Ehrlichia sp. (such as Ehrlichia chafeensia and Ehrlichia canis), Epidermophyton floccosum, Erysipelothrix rhusiopathiae, Eubacterium sp., Francisella tularensis, Fusobacterium nucleatum, Gardnerella vaginalis, Gemella morbillorum, Haemophilus sp. (such as Haemophilus influenzae, Haemophilus ducreyi, Haemophilus aegyptius, Haemophilus parainfluenzae, Haemophilus haemolyticus and Haemophilus parahaemolyticus), Helicobacter sp. (such as Helicobacter pylori, Helicobacter cinaedi and Helicobacter fennelliae), Kingella kingii, Klebsiella sp. (such as Klebsiella pneumoniae, Klebsiella granulomatis and Klebsiella oxytoca), Lactobacillus sp., Listeria monocytogenes, Leptospira interrogans, Legionella pneumophila, Leptospira interrogans, Peptostreptococcus sp., Mannheimia hemolytica, Microsporum canis, Moraxella catarrhalis, Morganella sp., Mobiluncus sp., Micrococcus sp., Mycobacterium sp. (such as Mycobacterium leprae, Mycobacterium tuberculosis, Mycobacterium paratuberculosis, Mycobacterium intracellulare, Mycobacterium avium, Mycobacterium bovis, and Mycobacterium marinum), Mycoplasm sp. (such as Mycoplasma pneumoniae, Mycoplasma hominis, and Mycoplasma genitalium), Nocardia sp. (such as Nocardia asteroides, Nocardia cyriacigeorgica and Nocardia brasiliensis), Neisseria sp. (such as Neisseria gonorrhoeae and Neisseria meningitidis), Pasteurella multocida, Pityrosporum orbiculare (Malassezia furfur), Plesiomonas shigelloides. Prevotella sp., Porphyromonas sp., Prevotella melaninogenica, Proteus sp. (such as Proteus vulgaris and Proteus mirabilis), Providencia sp. (such as Providencia alcalifaciens, Providencia rettgeri and Providencia stuartii), Pseudomonas aeruginosa, Propionibacterium acnes, Rhodococcus equi, Rickettsia sp. (such as Rickettsia rickettsii, Rickettsia akari and Rickettsia prowazekii, Orientia tsutsugamushi (formerly: Rickettsia tsutsugamushi) and Rickettsia typhi), Rhodococcus sp., Serratia marcescens, Stenotrophomonas maltophilia, Salmonella sp. (such as Salmonella enterica, Salmonella typhi, Salmonella paratyphi, Salmonella enteritidis, Salmonella cholerasuis and Salmonella typhimurium), Serratia sp. (such as Serratia marcesans and Serratia liquifaciens), Shigella sp. (such as Shigella dysenteriae, Shigella flexneri, Shigella boydii and Shigella sonnei), Staphylococcus sp. (such as Staphylococcus aureus, Staphylococcus epidermidis, Staphylococcus hemolyticus, Staphylococcus saprophyticus), Streptococcus sp. (such as Streptococcus pneumoniae (for example chloramphenicol-resistant serotype 4 Streptococcus pneumoniae, spectinomycin-resistant serotype 6B Streptococcus pneumoniae, streptomycin-resistant serotype 9V Streptococcus pneumoniae, erythromycin-resistant serotype 14 Streptococcus pneumoniae, optochin-resistant serotype 14 Streptococcus pneumoniae, rifampicin-resistant serotype 18C Streptococcus pneumoniae, tetracycline-resistant serotype 19F Streptococcus pneumoniae, penicillin-resistant serotype 19F Streptococcus pneumoniae, and trimethoprim-resistant serotype 23F Streptococcus pneumoniae, chloramphenicol-resistant serotype 4 Streptococcus pneumoniae, spectinomycin-resistant serotype 6B Streptococcus pneumoniae, streptomycin-resistant serotype 9V Streptococcus pneumoniae, optochin-resistant serotype 14 Streptococcus pneumoniae, rifampicin-resistant serotype 18C Streptococcus pneumoniae, penicillin-resistant serotype 19F Streptococcus pneumoniae, or trimethoprim-resistant serotype 23F Streptococcus pneumoniae), Streptococcus agalactiae, Streptococcus mutans, Streptococcus pyogenes, Group A streptococci, Streptococcus pyogenes, Group B streptococci, Streptococcus agalactiae, Group C streptococci, Streptococcus anginosus, Streptococcus equismilis, Group D streptococci, Streptococcus bovis, Group F streptococci, and Streptococcus anginosus Group G streptococci), Spirillum minus, Streptobacillus moniliformi, Treponema sp. (such as Treponema carateum, Treponema petenue, Treponema pallidum and Treponema endemicum), Trichophyton rubrum, T. mentagrophytes, Tropheryma whippelii, Ureaplasma urealyticum, Veillonella sp., Vibrio sp. (such as Vibrio cholerae, Vibrio parahemolyticus, Vibrio vulnificus, Vibrio parahaemolyticus, Vibrio vulnificus, Vibrio alginolyticus, Vibrio mimicus, Vibrio hollisae, Vibrio fluvialis, Vibrio metchnikovii, Vibrio damsela and Vibrio furnisii), Yersinia sp. (such as Yersinia enterocolitica, Yersinia pestis, and Yersinia pseudotuberculosis) and Xanthomonas maltophilia.

Examples of fungi include, but are not limited to, Aspergillus sp., Candida auris, Candida albicans, Candida dubliniensis, Candida famata, Candida glabrata, Candida guilliermondii, Candida kefyr, Candida lusitaniae, Candida krusei, Candida parapsilosis, Candida tropicalis, Cryptococcus gattii, Cryptococcus neoformans, Fusarium sp., Malassezia furfur, Rhodotorula sp., Trichosporon sp., Histoplasma capsulatum, Coccidioides immitis, and Pneumocystis carinii, as well as the causative agents of Apergillosis, Balsomycosis, Candidiasis, Coccidioidomycosis, fungal eye infections, fungal nail infections, histoplasmosis, mucormycosis, mycetoma, Pneuomcystis pneumonia, ringworm, sporotrichosis, crypococcosis, and Talaromycosis.

Examples of protozoan parasites include, but are not limited to, Plasmodium falciparum, P. vivax, P. ovals, P. malariae, P. berghei, Leishmania donovani, L. infantum, L. chagasi, L. mexicana, L. amazonensis, L. venezuelensis, L. tropica, L. major, L. minor, L. aethiopica, L. Biana braziliensis, L. (V.) guyanensis, L. (V.) panamensis, L. (V.) periviana, Trypanosoma brucei rhodesiense, T. brucei gambiense, T. cruzi, Giardia intestinalis, G. lamblia, Toxoplasma gondii, Entamoeba histolytica, Trichomonas vaginalis, Pneumocystis carinii, and Cryptosporidium parvum.

Examples of helminths include, but are not limited to, Filarioidea sp., Wuchereria sp. (such as Wuchereria bancrofti), Brugia sp. (such as Brugia malayi and Brugia timori), Loa sp. (such as Loa loa), Mansonella sp. (such as Mansonella streptocerca, Mansonella perstans, and Monsonella ozzardi), Onchocerca sp. (such as Onchocerca volvulus), Enterobius vermicularis, Ascaris sp. (such as Ascaris lumbricoides), Dracunculus (such as Dracunculus medinensis), Ancylostoma sp. (such as Ancylostoma duodenale, Ancylostoma braziliense, Ancylostoma tubaeforme, and Ancylostoma caninum), Necator sp. (such as Necator americanus), Trichuris sp. (such as Trichuris trichiura, Trichuris vulpis, Trichuris campanula, Trichuris suis, and Trichuris muris), Strongyloides sp. (such as Strongyloides stercoralis, Strongyloides canis, Strongyloides fuelleborni, Strongyloides cebus, and Strongyloides kellyi), Nematodirus sp., Moniezia sp., Oesophagostomum sp. (such as Oesophagostomum bifurcum, Oesophagostomum aculeatum, Oesophagostomum brumpti, Oesophagostomum stephanostomum, and Oesophagostomum stephanostomum var thomasi), Cooperia sp. (such as Cooperia ostertagi and Cooperia oncophora), Haemonchus sp., Ostertagia sp. (such as Ostertagia ostertagi), Trichostrongylus sp. (such as Trichostrongylus axei), Dirofilaria sp. (such as Dirofilaria immitis, Dirofilaria tenuis and Dirofilaria repens), and Schistosoma sp. (such as Schistosoma incognitum, Schistosoma ovuncatum, Schistosoma sinensium. Schistosoma indicum, Schistosoma nasale, Schistosoma spindale, Schistosoma japonicam, Schistosoma malayensis, Schistosoma mekongi, Schistosoma haematobium. Schistosoma bovis, Schistosoma curassoni, Schistosoma guineensis, Schistosoma haematobium, Schistosoma intercalatum, Schistosoma leiperi, Schistosoma margrebowiei, Schistosoma mattheei, Schistosoma mansoni, Schistosoma edwardiense, Schistosoma hippotami, and Schistosoma rodhaini)

Examples of viruses include, but are not limited to, disease-causing agents such as Adeno-associated virus, Aichi virus, Australian bat lyssavirus, BK polyomavirus, Banna virus, Barmah forest virus, Bunyamwera virus, Bunyavirus La Crosse, Bunyavirus snowshoe hare, Cercopithecine herpesvirus, Chandipura virus, Chikungunya virus, Coronavirus, Cosavirus A, Cowpox virus, Coxsackievirus, Crimean-Congo hemorrhagic fever virus, Dengue virus, Dhori virus, Dugbe virus, Duvenhage virus, Eastern equine encephalitis virus, Ebolavirus, Echovirus, Encephalomyocarditis virus, Epstein-Barr virus, European bat lyssavirus, GB virus C/Hepatitis G virus, Hantaan virus, Hendra virus, Hepatitis A virus, Hepatitis B virus, Hepatitis C virus, Hepatitis E virus, Hepatitis delta virus, Horsepox virus, Human adenovirus, Human astrovirus, Human coronavirus, Human cytomegalovirus, Human enterovirus 68, 70, Human herpesvirus 1, Human herpesvirus 2, Human herpesvirus 6, Human herpesvirus 7, Human herpesvirus 8, Human immunodeficiency virus, Human papillomavirus 1, Human papillomavirus 2, Human papillomavirus 16,18, Human parainfluenza, Human parvovirus B19, Human respiratory syncytial virus, Human rhinovirus, Human SARS coronavirus, Human spumaretrovirus, Human T-lymphotropic virus, Human torovirus, Influenza A virus, Influenza B virus, Influenza C virus, Isfahan virus, JC polyomavirus, Japanese encephalitis virus, Junin arenavirus, KI Polyomavirus, Kunjin virus, Lagos bat virus, Lake Victoria Marburgvirus, Langat virus, Lassa virus, Lordsdale virus, Louping ill virus, Lymphocytic choriomeningitis virus, Machupo virus, Mayaro virus, MERS coronavirus, Measles virus, Mengo encephalomyocarditis virus, Merkel cell polyomavirus, Mokola virus, Molluscum contagiosum virus, Monkeypox virus, Mumps virus, Murray valley encephalitis virus, New York virus, Nipah virus, Norwalk virus, Norovirus, O′nyong-nyong virus, Orf virus, Oropouche virus, Pichinde virus, Poliovirus, Punta toro phlebovirus, Puumala virus, Rabies virus, Rift valley fever virus, Rosavirus A, Ross river virus, Rotavirus A, Rotavirus B, Rotavirus C, Rubella virus, Sagiyama virus, Salivirus A, Sandfly fever sicilian virus, Sapporo virus, Semliki forest virus, Seoul virus, Severe acute respiratory syndrome coronavirus 2, Simian foamy virus, Simian virus 5, Sindbis virus, Southampton virus, St. louis encephalitis virus, Tick-borne powassan virus, Torque teno virus, Toscana virus, Uukuniemi virus, Vaccinia virus, Varicella-zoster virus, Variola virus, Venezuelan equine encephalitis virus, Vesicular stomatitis virus, Western equine encephalitis virus, WU polyomavirus, West Nile virus, Yaba monkey tumor virus, Yaba-like disease virus, Yellow fever virus, and Zika virus.

In some embodiments, the term “microorganism” will be understood to include any one or more bacteria, fungi, protozoa, viruses, algac, archaea, phages, and/or helminths represented in a database (e.g., a microbial genome database, a transcriptomic database, a proteomic database, a metabolomics database, a taxonomic database, and/or a clinical database). In some embodiments, the database comprises one or more entries corresponding to and/or identifying a microorganism (e.g., an annotation, for a respective microorganism, to a genome, transcriptome, nucleic acid sequence, protein sequence, metabolite, taxonomic record and/or clinical record). In some embodiments, a microorganism is in a national and/or international database. Examples of such databases include, but are not limited to, NCBI, BLAST, EMBL-EBI, GenBank, Ensembl, EuPathDB, The Human Microbiome Project, Pathogen Portal, RDP, SILVA, GREENGENES, EBI Metagenomics, EcoCyc, PATRIC, TBDB, PlasmoDB, the Microbial Genome Database (MBGD), and/or the Microbial Rosetta Stone Database. For example, MBGD comprises all complete genome sequences of bacteria, archaea, and unicellular eukaryotes, including fungi and protozoa, available at the NCBI genomes site. The Microbial Rosetta Stone is a database that provides information on disease-causing organisms (e.g., bacteria, fungi, protozoa, DNA viruses, RNA viruses, plants, and animals) and the toxins produced therefrom. See, Zhulin, 2015, “Databases for Microbiologists,” J Bacteriol 197:2458-2467, doi: 10.1128/JB.00330-15; Uchiyama et al., 2019, “MBGD update 2018: microbial genome database based on hierarchical orthology relations covering closely related and distantly related comparisons,” Nuc Acids Res., 47 (D1), D382-D389, doi: 10.1093/nar/gkyl054; and Ecker et al., 2005, “The Microbial Rosetta Stone Database: A compilation of global and emerging infectious microorganisms and bioterrorist threat agents,” BMC Microbiology 5, 19, doi: 10.1186/1471-2180-5-19; each of which is hereby incorporated by reference herein in its entirety.

In some embodiments, a microorganism is a commensal organism (e.g., is commonly associated with the source or site of sample collection and/or is not considered to be harmful). For example, hundreds of microorganisms are known to co-exist in the oral microbiome, and their existence in a sample collected from the oral cavity of a subject may not be indicative of a disease state. In some embodiments, a microorganism exists in a symbiotic (e.g., endosymbiotic) relationship with a subject (e.g., a host organism). In some embodiments, a microorganism is considered healthy, normal, and/or beneficial to health, such as a probiotic. Other suitable alternatives include various microorganisms that are known or have been shown to contribute to immune health, synthesize useful vitamins, and/or ferment indigestible carbohydrates.

In some embodiments, a microorganism is a pathogen (e.g., disease-causing), such as a human, animal, or plant-infective pathogen. In some embodiments, a microorganism is associated with a disease and/or is known or has been shown to be otherwise harmful to a population, such as a human population. For example, in some embodiments, a microorganism is a pathogen that is a causative agent in an infectious disease. In some embodiments, a microorganism is present in a sample (e.g., a subject, source and/or site of collection) at an asymptomatic level (e.g., at a level unlikely to induce disease or infection). In some embodiments, a microorganism is present in a sample (e.g., a subject, source and/or site of collection) at a symptomatic level (e.g., a chronic and/or acute symptomatic level).

Model. In some embodiments, as used herein, the term “model” refers to a machine learning model or algorithm. In some embodiments, a model is supervised machine learning. Nonlimiting examples of supervised learning algorithms include, but are not limited to, logistic regression, neural networks, support vector machines, Naive Bayes algorithms, nearest neighbor algorithms, random forest algorithms, decision tree algorithms, boosted trees algorithms, multinomial logistic regression algorithms, linear models, linear regression, GradientBoosting, mixture models, hidden Markov models, Gaussian NB algorithms, linear discriminant analysis, or any combinations thereof. In some embodiments, a model is a multinomial classifier algorithm. In some embodiments, a model is a 2-stage stochastic gradient descent (SGD) model. In some embodiments, a model is a deep neural network (e.g., a deep-and-wide sample-level classifier). In some embodiments, a model comprises 100 or more, 1000 or more, 10,000 or more, 100,000 or more or 1×10⁶ or more parameters.

Parameter. Moreover, as used herein, the term “parameter” refers to any coefficient or, similarly, any value of an internal or external element (e.g., a weight and/or a hyperparameter) in an algorithm, model, regressor, and/or classifier that can affect (e.g., modify, tailor, and/or adjust) one or more inputs, outputs, and/or functions in the algorithm, model, regressor and/or classifier. For example, in some embodiments, a parameter refers to any coefficient, weight, and/or hyperparameter that can be used to control, modify, tailor, and/or adjust the behavior, learning, and/or performance of an algorithm, model, regressor, and/or classifier. In some instances, a parameter is used to increase or decrease the influence of an input (e.g., a feature) to an algorithm, model, regressor, and/or classifier. As a nonlimiting example, in some embodiments, a parameter is used to increase or decrease the influence of a node (e.g., of a neural network), where the node includes one or more activation functions. Assignment of parameters to specific inputs, outputs, and/or functions is not limited to any one paradigm for a given algorithm, model, regressor, and/or classifier but can be used in any suitable algorithm, model, regressor, and/or classifier architecture for a desired performance. In some embodiments, a parameter has a fixed value. In some embodiments, a value of a parameter is manually and/or automatically adjustable. In some embodiments, a value of a parameter is modified by a validation and/or training process for an algorithm, model, regressor, and/or classifier (e.g., by error minimization and/or backpropagation methods). In some embodiments, an algorithm, model, regressor, and/or classifier of the present disclosure includes a plurality of parameters. In some embodiments, the plurality of parameters is n parameters, where: n≥2; n≥5; n≥10; n≥25; n≥40; n≥50; n≥75; n≥100; n≥125; n≥150; n≥200; n≥225; n≥250; n≥350; n≥500; n≥600; n≥750; n≥1,000; n≥2,000; n≥4,000; n≥5,000; n≥7,500; n≥10,000; n≥20,000; n≥40,000; n≥75,000; n≥100,000; n≥200,000; n≥500,000, n≥1×10⁶, n≥5×10⁶, or n≥1×10⁷. In some embodiments n is between 10,000 and 1×10⁷, between 100,000 and 5×10⁶, or between 500,000 and 1×10⁶. In some embodiments, the algorithms, models, regressors, and/or classifier of the present disclosure operate in a k-dimensional space, where k is a positive integer of 5 or greater (e.g., 5, 6, 7, 8, 9, 10, etc.). As such, the algorithms, models, regressors, and/or classifiers of the present disclosure cannot be mentally performed.

Sequence Read and/or Read. As used herein, the term “sequence read” or “read” refers to a sequence read from a portion of a nucleic acid sample. Typically, though not necessarily, a read represents a short sequence of contiguous base pairs in the sample. In some embodiments, a read is represented symbolically by the base pair sequence (in ATCG) of the sample portion. In some cases, a read is stored in a memory device and processed as appropriate to determine whether it matches a reference sequence or meets other criteria. In some instances, a read is obtained directly from a sequencing apparatus or indirectly from stored sequence information concerning the sample. In some cases, a read is a DNA sequence of sufficient length (e.g., at least about 25 bp) that can be used to identify a larger sequence or region, e.g., that can be aligned and mapped to a chromosome or genomic region or gene.

In some embodiments, sequence reads are produced by any sequencing process described herein or known in the art. In some cases, reads are generated from one end of nucleic acid fragments (“single-end reads”) or from both ends of nucleic acids (e.g., paired-end reads, double-end reads). The length of the sequence read is often associated with the particular sequencing technology. High-throughput methods, for example, provide sequence reads that can vary in size from tens to hundreds of base pairs (bp). In some embodiments, the sequence reads are of a mean, median or average length of about 15 bp to 900 bp long (e.g., about 20 bp, about 25 bp, about 30 bp, about 35 bp, about 40 bp, about 45 bp, about 50 bp, about 55 bp, about 60 bp, about 65 bp, about 70 bp, about 75 bp, about 80 bp, about 85 bp, about 90 bp, about 95 bp, about 100 bp, about 110 bp, about 120 bp, about 130, about 140 bp, about 150 bp, about 200 bp, about 250 bp, about 300 bp, about 350 bp, about 400 bp, about 450 bp, or about 500 bp. In some embodiments, the sequence reads are of a mean, median or average length of about 1000 bp, 2000 bp, 5000 bp, 10,000 bp, or 50,000 bp or more. Nanopore sequencing, for example, can provide sequence reads that can vary in size from tens to hundreds to thousands of base pairs. Illumina parallel sequencing can provide sequence reads that do not vary as much, for example, most of the sequence reads can be smaller than 200 bp. In some embodiments, sequence reads are obtained in a variety of ways, e.g., using sequencing techniques or using probes, e.g., in hybridization arrays or capture probes, or amplification techniques, such as the polymerase chain reaction (PCR) or linear amplification using a single primer or isothermal amplification.

Subject. As used herein, the term “subject” refers to a human subject as well as a non-human subject such as a mammal, an invertebrate, a vertebrate, a fungus, a yeast, a bacterium, and a virus. Although the examples herein concern humans and the language is primarily directed to human concerns, the concepts disclosed herein are applicable to genomes from any plant or animal, and are useful in the fields of veterinary medicine, animal sciences, research laboratories and such.

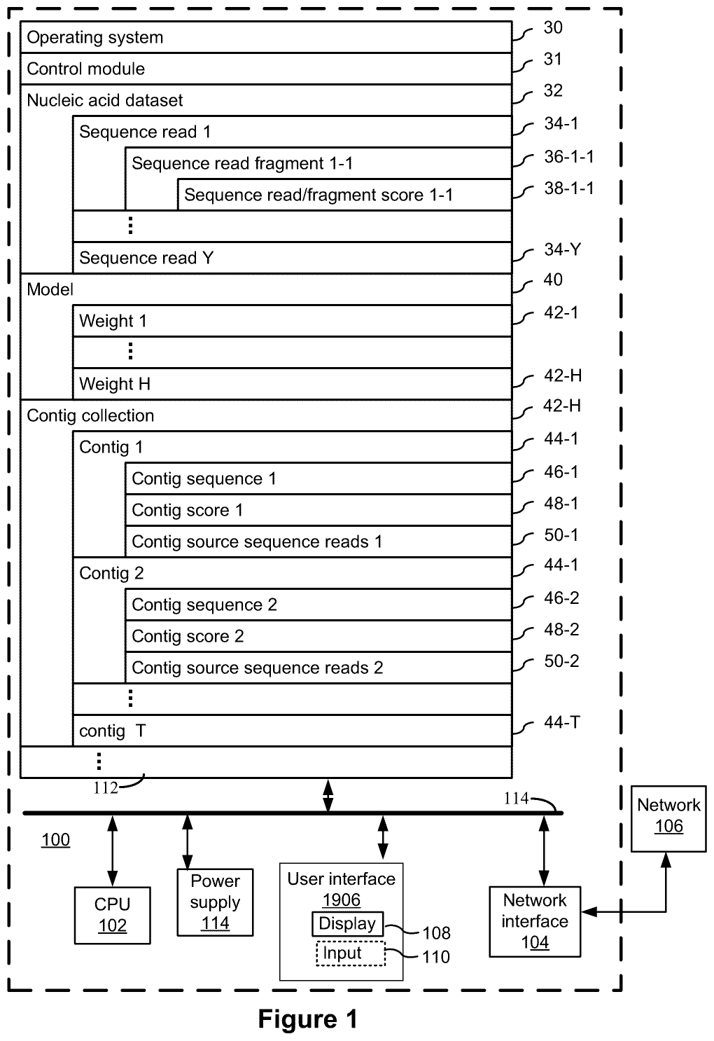

EXAMPLE SYSTEM. In the present disclosure, unless expressly stated otherwise, descriptions of devices and systems will include implementations of one or more computers. For instance, and for purposes of illustration in FIG. 1, a computer system 100 is represented as single device that includes all the functionality of the computer system 100. However, the present disclosure is not limited thereto. For instance, in some embodiments, the functionality of the computer system 100 is spread across any number of networked computers and/or reside on each of several networked computers and/or by hosted on one or more virtual machines and/or containers at a remote location accessible across a communications network (e.g., communications network 106 of FIG. 1). One of skill in the art will appreciate that a wide array of different computer topologies is possible for the computer system 100, and other devices and systems of the preset disclosure, and that all such topologies are within the scope of the present disclosure. Moreover, rather than relying on a physical communications network 106, the illustrated devices and systems may wirelessly transmit information between each other. As such, the exemplary topology shown in FIG. 1 merely serves to describe the features of an embodiment of the present disclosure in a manner that will be readily understood to one of skill in the art.

FIG. 1 depicts a block diagram of a distributed computer system (e.g., computer system 100) according to some embodiments of the present disclosure. The computer system 100 at least facilitates communicating one or more instructions for identifying a viral sequence in a subject of a species.

In some embodiments, the communication network 106 optionally includes the Internet, one or more local area networks (LANs), one or more wide area networks (WANs), other types of networks, or a combination of such networks.

Examples of communication networks 106 include the World Wide Web (WWW), an intranet and/or a wireless network, such as a cellular telephone network, a wireless local area network (LAN) and/or a metropolitan area network (MAN), and other devices by wireless communication. The wireless communication optionally uses any of a plurality of communications standards, protocols and technologies, including Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VOIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS), or any other suitable communication protocol, including communication protocols not yet developed as of the filing date of this document.

In various embodiments, the computer system 100 includes one or more processing units (CPUs, processing cores, etc.) 102, a network or other communications interface 104, and memory 112.

In some embodiments, the computer system 100 includes a user interface 106. The user interface 106 typically includes a display 108 for presenting media. In some embodiments, the display 108 is integrated within the computer systems (e.g., housed in the same chassis as the CPU 102 and memory 112). In some embodiments, the computer system 100 includes one or more input device(s) 110, which allow a subject to interact with the computer system 100. In some embodiments, input devices 110 include a keyboard, a mouse, and/or other input mechanisms. Alternatively, or in addition, in some embodiments, the display 108 includes a touch-sensitive surface (e.g., where display 108 is a touch-sensitive display or computer system 100 includes a touch pad).

In some embodiments, the computer system 100 presents media to a user through the display 108. Examples of media presented by the display 108 include one or more images, a video, audio (e.g., waveforms of an audio sample), or a combination thereof. In typical embodiments, the one or more images, the video, the audio, or the combination thereof is presented by the display 108 through a client application. In some embodiments, the audio is presented through an external device (e.g., speakers, headphones, input/output (I/O) subsystem, etc.) that receives audio information from the computer system 100 and presents audio data based on this audio information. In some embodiments, the user interface 106 also includes an audio output device, such as speakers or an audio output for connecting with speakers, earphones, or headphones.

Memory 112 includes high-speed random access memory, such as DRAM, SRAM, DDR RAM, or other random access solid state memory devices, and optionally also includes non-volatile memory, such as one or more magnetic disk storage devices, optical disk storage devices, flash memory devices, or other non-volatile solid state storage devices. Memory 112 may optionally include one or more storage devices remotely located from the CPU(s) 102. Memory 112, or alternatively the non-volatile memory device(s) within memory 112, includes a non-transitory computer readable storage medium. Access to memory 112 by other components of the computer system 100, such as the CPU(s) 102, is, optionally, controlled by a controller. In some embodiments, the memory 112 include mass storage that is remotely located with respect to the CPU(s) 102. In other words, some data stored in memory 112 may in fact be hosted on devices that are external to the computer system 100, but that can be electronically accessed by the computer system 100 over an Internet, intranet, or other form of network 106 or electronic cable using communication interface 104.

In some embodiments, the memory 112 of the computer system 100 for identifying a viral sequence in a subject of a species stores:
an optional operating system 30 (e.g., ANDROID, IOS, DARWIN, RTXC, LINUX, UNIX, OS X, WINDOWS, or an embedded operating system such as VxWorks) that includes procedures for handling various basic system services;
a control module 31 for controlling one or more processes (e.g., method) associated with the computer system 100;
a nucleic acid dataset 32 comprising a plurality of sequences reads 34, optionally fragmented 36 along with a corresponding score 38;
a model 40 comprising a plurality of weights 42; and
a contig collection 42, each contig 44 in the contig collection including a contig sequence 46, a contig score, and the identity 50 of the sequence reads used to form the contig.

In some embodiments, the control module 31 includes one or more models that is configured to perform one or more steps of a method of the present disclosure.

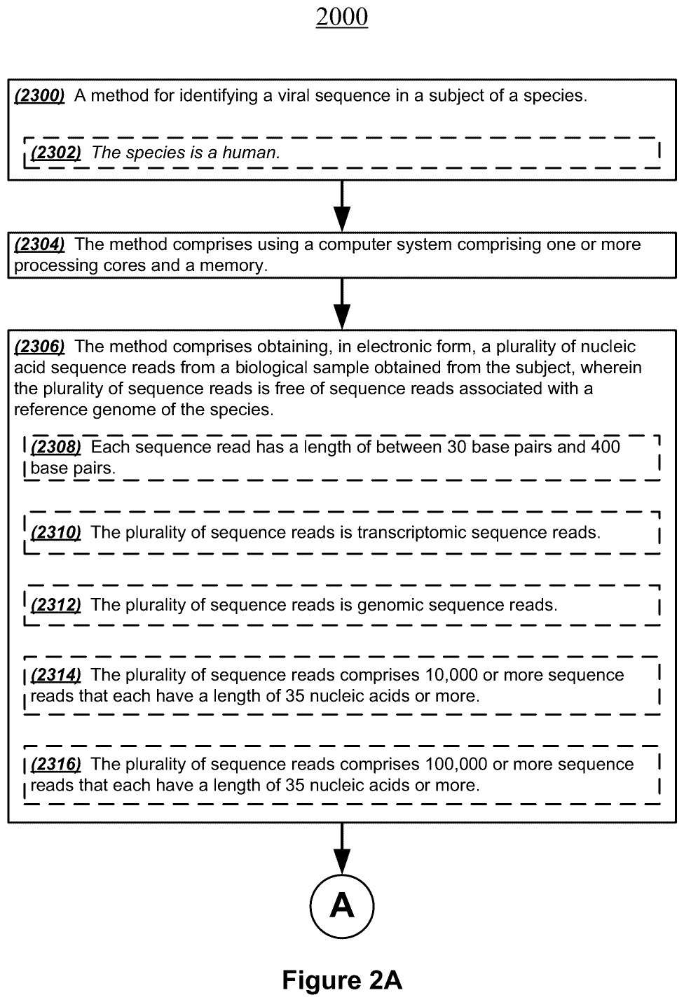

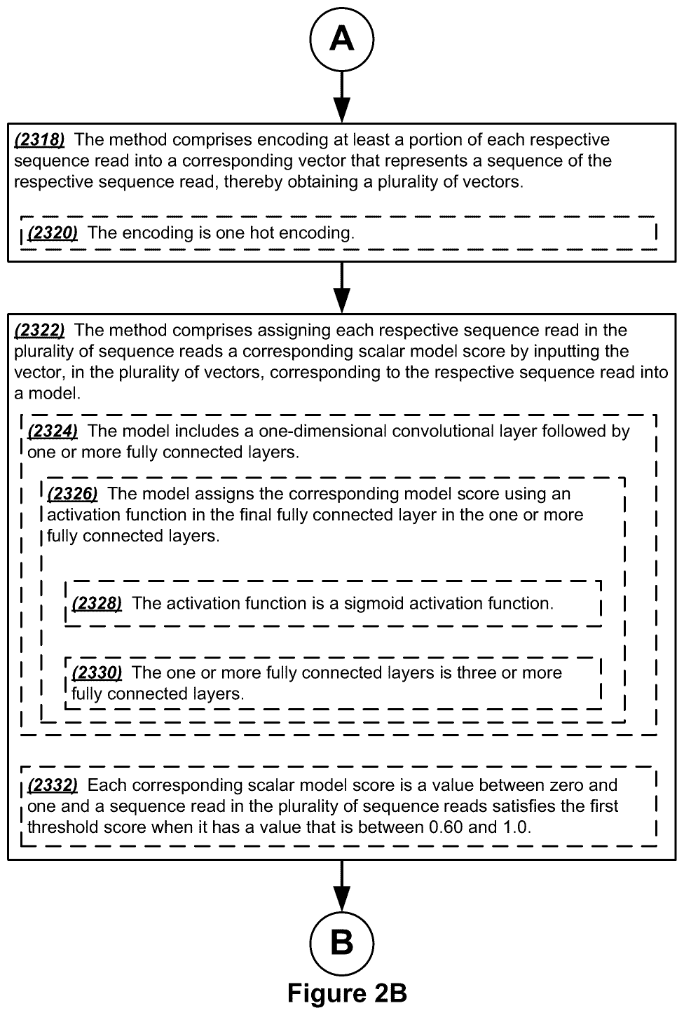

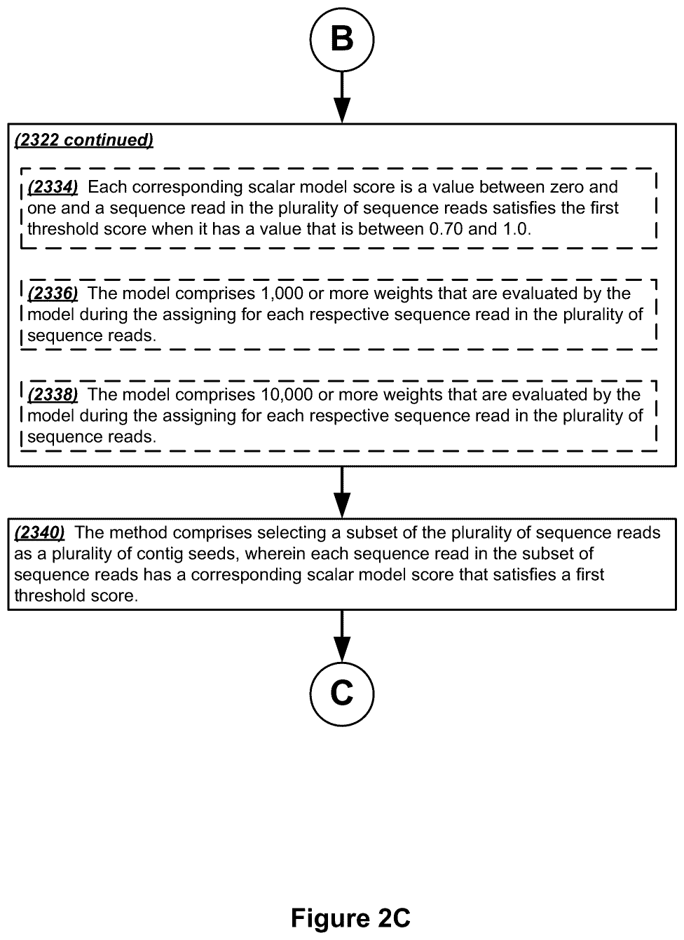

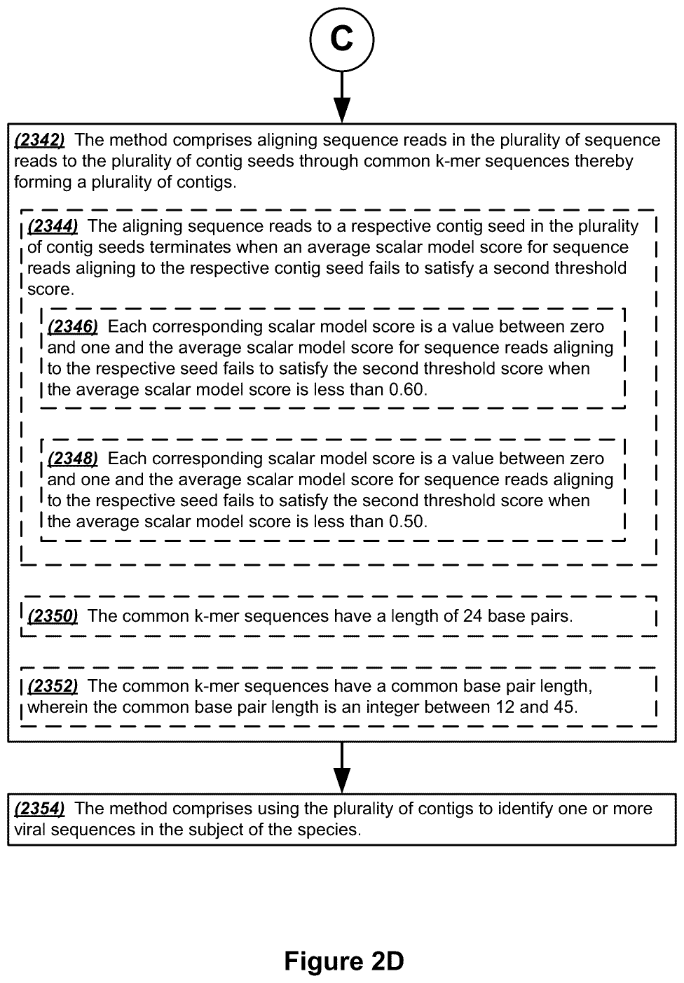

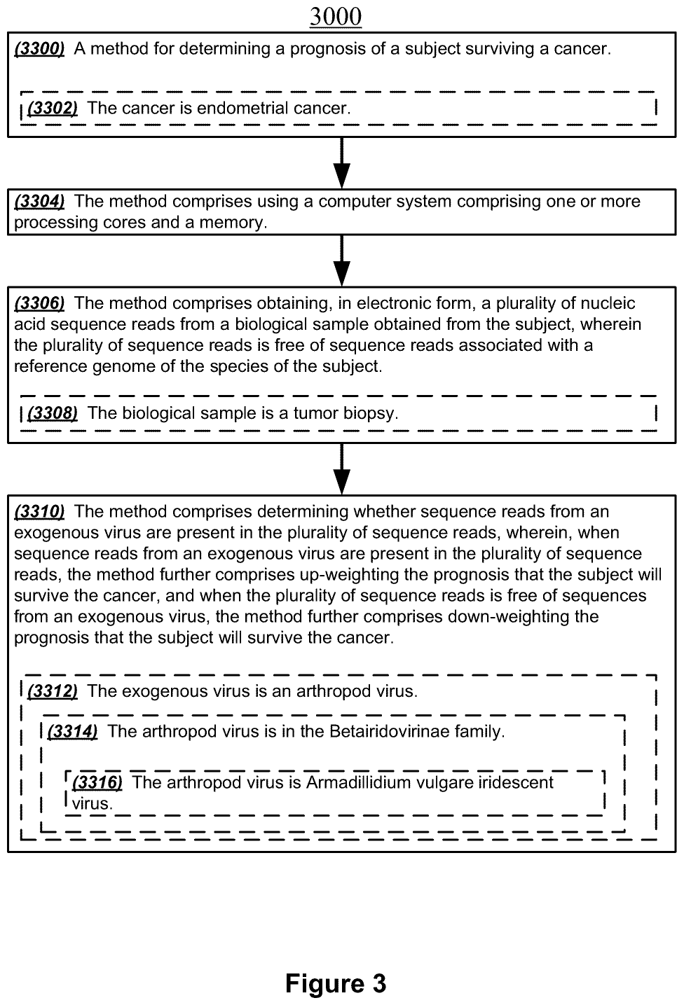

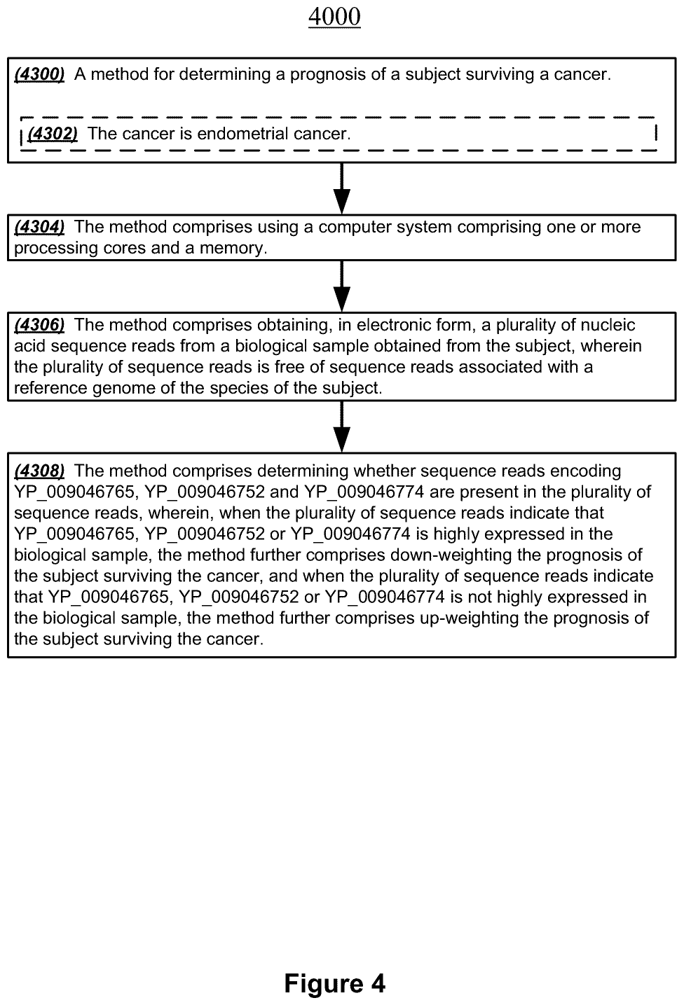

Each of the above identified modules and applications correspond to a set of executable instructions for performing one or more functions described above and the methods described in the present disclosure (e.g., the computer-implemented methods and other information processing methods described herein, method 2000 of FIGS. 2A through 2D, method 3000 of FIG. 3, method 4000 of FIG. 4, etc.). These modules (e.g., sets of instructions) need not be implemented as separate software programs, procedures or modules, and thus various subsets of these modules are, optionally, combined or otherwise re-arranged in various embodiments of the present disclosure. In some embodiments, the memory 112 optionally stores a subset of the modules and data structures identified above. Furthermore, in some embodiments, the memory 112 stores additional modules and data structures not described above.

It should be appreciated that the computer system of FIG. 1 is only one example of a computer system 100, and that the computer system 100 optionally has more or fewer components than shown, optionally combines two or more components, or optionally has a different configuration or arrangement of the components. The various components shown in FIG. 1 are implemented in hardware, software, firmware, or a combination thereof, including one or more signal processing and/or application specific integrated circuits.

EXAMPLE METHODS. Now that a general topology of the computer system 100 has been described in accordance with various embodiments of the present disclosures, details regarding some processes in accordance with FIGS. 2A, 2B, 2C, 2D, 3, and 4 will be described.

FIGS. 2A, 2B, 2C, and 2D collectively illustrates a flow chart of methods (e.g., method 2000) for identifying a viral sequence in a subject of a species, in accordance with embodiments of the present disclosure. Specifically, an exemplary method 2000 for identifying a viral sequence in a subject of a species is provided, in accordance with some embodiments of the present disclosure. In the flow charts, the preferred parts of the methods are shown in solid line boxes, whereas optional variants of the methods, or optional equipment used by the methods, are shown in dashed line boxes. As such, FIGS. 2A through 2D collectively illustrate methods for identifying a viral sequence in a subject of a species.

Various modules in the memory 112 of the computer system 100 perform certain processes of the methods described in FIGS. 2A through 2D, unless expressly stated otherwise. Furthermore, it will be appreciated that the processes in FIGS. 2A through 2D can be encoded in a single module or any combination of modules.

Block 2300. Referring to block 2300 of FIG. 2A, a method for identifying a viral sequence in a subject of a species is provided. In alternative embodiments, rather than a viral sequence, the sequence is the sequence of any microorganism in a host species. For instance, in some embodiments the microorganism is a bacteria, fungi, protozoa, viruses, algae, archaea, phages, or helminth and the host species is an animal or a plant. In some embodiments the microorganism is a bacteria, fungi, protozoa, viruses, algae, archaea, phages, or helminth and the host species is a mammal. In some embodiments the microorganism is a bacteria, fungi, protozoa, viruses, algae, archaea, phages, or helminth and the host species is human.

Block 2302. Referring to block 2302, in some embodiments, the species is human. As such, any viral sequence found using the systems and methods of the present disclosure in such embodiments would represent an infection of the human by the virus associated with the viral sequence.

Block 2304. Referring to block 2304, in some embodiments, the method includes using a computer system (e.g., computer system 100 of FIG. 1). The computer system includes one or more processing cores (e.g., CPU 102 of FIG. 1) and a memory (e.g., memory 112 of FIG. 1).

Block 2306. Referring to block 2306, in some embodiments, the method includes obtaining, in electronic form, a plurality of nucleic acid sequence reads from a biological sample obtained from the subject. In some embodiments, the plurality of sequence reads is free of sequence reads associated with a reference genome of the species. For instance, in some embodiments, the plurality of nucleic acid sequence reads are mapped using a mapping program, such as Bowtie2 against hg19 (1000 Genomes version) and PhiX phage (NC_001422), and only the unmapped reads are kept in the plurality of nucleic acid sequence reads.

In some embodiments, the biological sample is derived from a biological fluid, cell, tissue, organ, or organism, that includes a nucleic acid or a mixture of nucleic acids having at least one nucleic acid sequence that is to be assayed. Such samples include, but are not limited to sputum/oral fluid, amniotic fluid, blood, blood fractions, fine needle biopsy samples, urine, peritoneal fluid, pleural fluid, and the like. In some embodiments, the biological sample is used directly as obtained from the subject or following a pretreatment to modify the character of the sample. For example, in some implementations, such pretreatment includes preparing plasma from blood, diluting viscous fluids, and so forth. In some embodiments, methods of pretreatment also involve, but are not limited to, filtration, precipitation, dilution, distillation, mixing, centrifugation, freezing, lyophilization, concentration, amplification, nucleic acid fragmentation, inactivation of interfering components, the addition of reagents, and/or lysing. If such methods of pretreatment are employed with respect to the biological sample, such pretreatment methods are typically such that the nucleic acid(s) of interest remain in the biological sample, sometimes at a concentration proportional to that in an untreated biological sample (e.g., namely, a sample that is not subjected to any such pretreatment method(s)). Such “treated” or “processed” biological samples are still considered to be biological samples with respect to the methods described herein.

Block 2308. Referring to block 2308, in some embodiments, each sequence read has a length of between 30 base pairs and 400 base pairs. In some embodiments, each sequence reads has a length of 40 base pairs.

In some embodiments, each sequence read has a length of between 25 base pairs and 450 base pairs, between 25 base pairs and 425 base pairs, between 25 base pairs and 400 base pairs, between 25 base pairs and 375 base pairs, between 25 base pairs and 350 base pairs, between 25 base pairs and 325 base pairs, between 25 base pairs and 300 base pairs, between 25 base pairs and 275 base pairs, between 25 base pairs and 250 base pairs, between 25 base pairs and 225 base pairs, between 25 base pairs and 200 base pairs, between 30 base pairs and 450 base pairs, between 30 base pairs and 425 base pairs, between 30 base pairs and 400 base pairs, between 30 base pairs and 375 base pairs, between 30 base pairs and 350 base pairs, between 30 base pairs and 325 base pairs, between 30 base pairs and 300 base pairs, between 30 base pairs and 275 base pairs, between 30 base pairs and 250 base pairs, between 30 base pairs and 225 base pairs, between 30 base pairs and 200 base pairs, between 35 base pairs and 450 base pairs, between 35 base pairs and 425 base pairs, between 35 base pairs and 400 base pairs, between 35 base pairs and 375 base pairs, between 35 base pairs and 350 base pairs, between 35 base pairs and 325 base pairs, between 35 base pairs and 300 base pairs, between 35 base pairs and 275 base pairs, between 35 base pairs and 250 base pairs, between 35 base pairs and 225 base pairs, between 35 base pairs and 200 base pairs, between 40 base pairs and 450 base pairs, between 40 base pairs and 425 base pairs, between 40 base pairs and 400 base pairs, between 40 base pairs and 375 base pairs, between 40 base pairs and 350 base pairs, between 40 base pairs and 325 base pairs, between 40 base pairs and 300 base pairs, between 40 base pairs and 275 base pairs, between 40 base pairs and 250 base pairs, between 40 base pairs and 225 base pairs, between 40 base pairs and 200 base pairs, between 50 base pairs and 450 base pairs, between 50 base pairs and 425 base pairs, between 50 base pairs and 400 base pairs, between 50 base pairs and 375 base pairs, between 50 base pairs and 350 base pairs, between 50 base pairs and 325 base pairs, between 50 base pairs and 300 base pairs, between 50 base pairs and 275 base pairs, between 50 base pairs and 250 base pairs, between 50 base pairs and 225 base pairs, or between 50 base pairs and 200 base pairs.

In some embodiments, each sequence read has a length of at least 25 base pairs, at least 30 base pairs, at least 35 base pairs, at least 40 base pairs, at least 45 base pairs, at least 50 base pairs, at least 55 base pairs, at least 60 base pairs, at least 65 base pairs, at least 70 base pairs, at least 75 base pairs, at least 80 base pairs, at least 85 base pairs, at least 90 base pairs, at least 95 base pairs, at least 100 base pairs, at least 105 base pairs, at least 110 base pairs, at least 115 base pairs, at least 120 base pairs, at least 125 base pairs, at least 130 base pairs, at least 135 base pairs, at least 140 base pairs, at least 145 base pairs, at least 150 base pairs, at least 155 base pairs, at least 160 base pairs, at least 165 base pairs, at least 170 base pairs, at least 175 base pairs, at least 180 base pairs, at least 185 base pairs, at least 190 base pairs, at least 195 base pairs, at least 200 base pairs, at least 205 base pairs, at least 210 base pairs, at least 215 base pairs, at least 220 base pairs, at least 225 base pairs, at least 230 base pairs, at least 235 base pairs, at least 240 base pairs, at least 245 base pairs, at least 250 base pairs, at least 255 base pairs, at least 260 base pairs, at least 265 base pairs, at least 270 base pairs, at least 275 base pairs, at least 280 base pairs, at least 285 base pairs, at least 290 base pairs, at least 295 base pairs, at least 300 base pairs, at least 305 base pairs, at least 310 base pairs, at least 315 base pairs, at least 320 base pairs, at least 325 base pairs, at least 330 base pairs, at least 335 base pairs, at least 340 base pairs, at least 345 base pairs, at least 350 base pairs, at least 355 base pairs, at least 360 base pairs, at least 365 base pairs, at least 370 base pairs, at least 375 base pairs, at least 380 base pairs, at least 385 base pairs, at least 390 base pairs, at least 395 base pairs, at least 400 base pairs, at least 405 base pairs, at least 410 base pairs, at least 415 base pairs, at least 420 base pairs, at least 425 base pairs, at least 430 base pairs, at least 435 base pairs, at least 440 base pairs, at least 445 base pairs, or at least 450 base pairs. In some embodiments, each sequence read has a length of at most 25 base pairs, at most 30 base pairs, at most 35 base pairs, at most 40 base pairs, at most 45 base pairs, at most 50 base pairs, at most 55 base pairs, at most 60 base pairs, at most 65 base pairs, at most 70 base pairs, at most 75 base pairs, at most 80 base pairs, at most 85 base pairs, at most 90 base pairs, at most 95 base pairs, at most 100 base pairs, at most 105 base pairs, at most 110 base pairs, at most 115 base pairs, at most 120 base pairs, at most 125 base pairs, at most 130 base pairs, at most 135 base pairs, at most 140 base pairs, at most 145 base pairs, at most 150 base pairs, at most 155 base pairs, at most 160 base pairs, at most 165 base pairs, at most 170 base pairs, at most 175 base pairs, at most 180 base pairs, at most 185 base pairs, at most 190 base pairs, at most 195 base pairs, at most 200 base pairs, at most 205 base pairs, at most 210 base pairs, at most 215 base pairs, at most 220 base pairs, at most 225 base pairs, at most 230 base pairs, at most 235 base pairs, at most 240 base pairs, at most 245 base pairs, at most 250 base pairs, at most 255 base pairs, at most 260 base pairs, at most 265 base pairs, at most 270 base pairs, at most 275 base pairs, at most 280 base pairs, at most 285 base pairs, at most 290 base pairs, at most 295 base pairs, at most 300 base pairs, at most 305 base pairs, at most 310 base pairs, at most 315 base pairs, at most 320 base pairs, at most 325 base pairs, at most 330 base pairs, at most 335 base pairs, at most 340 base pairs, at most 345 base pairs, at most 350 base pairs, at most 355 base pairs, at most 360 base pairs, at most 365 base pairs, at most 370 base pairs, at most 375 base pairs, at most 380 base pairs, at most 385 base pairs, at most 390 base pairs, at most 395 base pairs, at most 400 base pairs, at most 405 base pairs, at most 410 base pairs, at most 415 base pairs, at most 420 base pairs, at most 425 base pairs, at most 430 base pairs, at most 435 base pairs, at most 440 base pairs, at most 445 base pairs, or at most 450 base pairs.

Block 2310. Referring to block 2310, in some embodiments, the plurality of sequence reads is transcriptomic sequence reads. For example, in some embodiments the plurality of sequence reads is a whole transcriptome RNA-seq panel.

Block 2312. Referring to block 2312, in some embodiments, the plurality of sequence reads is genomic sequence reads.

Blocks 2314-2316. Referring to blocks 2314-2316, in some embodiments, the plurality of sequence reads includes 10,000 or more sequence reads that each have a length of 35 nucleic acids or more.

In some embodiments, the plurality of sequence reads includes 5,000 or more sequence reads, 10,000 or more sequence reads, 15,000 or more sequence reads, 20,000 or more sequence reads, 25,000 or more sequence reads, 30,000 or more sequence reads, 35,000 or more sequence reads, 40,000 or more sequence reads, 45,000 or more sequence reads, 50,000 or more sequence reads, 55,000 or more sequence reads, 60,000 or more sequence reads, 65,000 or more sequence reads, 70,000 or more sequence reads, 75,000 or more sequence reads, 80,000 or more sequence reads, 85,000 or more sequence reads, 90,000 or more sequence reads, 95,000 or more sequence reads, 100,000 or more sequence reads, 105,000 or more sequence reads, 110,000 or more sequence reads, or 115,000 or more sequence reads that each have a length of 30 nucleic acids or more.

In some embodiments, the plurality of sequence reads includes 5,000 or more sequence reads, 10,000 or more sequence reads, 15,000 or more sequence reads, 20,000 or more sequence reads, 25,000 or more sequence reads, 30,000 or more sequence reads, 35,000 or more sequence reads, 40,000 or more sequence reads, 45,000 or more sequence reads, 50,000 or more sequence reads, 55,000 or more sequence reads, 60,000 or more sequence reads, 65,000 or more sequence reads, 70,000 or more sequence reads, 75,000 or more sequence reads, 80,000 or more sequence reads, 85,000 or more sequence reads, 90,000 or more sequence reads, 95,000 or more sequence reads, 100,000 or more sequence reads, 105,000 or more sequence reads, 110,000 or more sequence reads, or 115,000 or more sequence reads that each have a length of 35 nucleic acids or more.

In some embodiments, the plurality of sequence reads includes 5,000 or more sequence reads, 10,000 or more sequence reads, 15,000 or more sequence reads, 20,000 or more sequence reads, 25,000 or more sequence reads, 30,000 or more sequence reads, 35,000 or more sequence reads, 40,000 or more sequence reads, 45,000 or more sequence reads, 50,000 or more sequence reads, 55,000 or more sequence reads, 60,000 or more sequence reads, 65,000 or more sequence reads, 70,000 or more sequence reads, 75,000 or more sequence reads, 80,000 or more sequence reads, 85,000 or more sequence reads, 90,000 or more sequence reads, 95,000 or more sequence reads, 100,000 or more sequence reads, 105,000 or more sequence reads, 110,000 or more sequence reads, or 115,000 or more sequence reads that each have a length of 40 nucleic acids or more.

Block 2318. Referring to block 2318 of FIG. 2B, in some embodiments, the method includes encoding at least a portion of each respective sequence read into a corresponding vector that represents a sequence of the respective sequence read. In some embodiments this vector takes the form of a sequence read fragment 36. Accordingly, the disclosed systems and methods obtain a plurality of vectors, where each such vector contains all or a portion of a corresponding sequence read. The encoding is used to ensure that each input into model 40 has a fixed length (e.g., 40 base parts, etc.). In some embodiments, the sequence reads are 40 base pair sequence reads and the model inputs 40 base pair sequence reads. In such embodiments, the encoding stores the name of each residue of the sequence read in a corresponding element in a 40 element vector. In some embodiments, the sequence read is less than the fixed input of the model 40. As an example, consider a sequence read that is 37 residues long and a model that requires a 40 residue input. In this example, the encoding encodes the 37 residues into the first 37 elements of a 40 element vector and the remaining 3 terminal elements are coded as missing. In some embodiments, the sequence read 34 is longer than the fixed input field of the model. In some such embodiments, the encoding segments the sequence read into sequence read fragments 36, where each sequence read fragment 36 is the length (in sequence read base pairs) required for model input. In some such embodiments, each sequence read fragment 35 is treated as a sequence read for purposes of model scoring and contig creation. In some embodiments, the segmentation is performed using a segmentation window. For example, in some embodiments the segmentation window is advanced after segmentation by the segmentation length. In an example where the segmentation window is 40 residues and the advance is 40 residues, the first forty residues of the sequence read 34 are placed in a first sequence read fragment 36 by the encoding, the second forty residues (starting just after the first forty residues) in the sequence read are placed in a second sequence read fragment 36 by the encoding, and so forth. Each of these sequence read fragments can be considered a vector that is individually scored by the model 40 and used to look for contigs 42.

In the case where the segmentation window is advanced by a distance that is the same as the segmentation length, such as in the example above, each sequence read fragment 36 corresponding to a sequence read 34 represents a unique portion of the sequence read. In embodiments where the segmentation window is advanced by a length that is smaller than the segmentation length, it is possible for sequence read fragments to have overlapping sequences from the sequence read. For instance, in the case where the sequence read is 100 residue long, the segmentation length (segmentation window) is forty residues, and the segmentation advance is two residues, residues 1-40 are segmented into a first sequence read fragment 36, residues 2-42 are segmented into a second sequence read fragment 36, residues 4-44 are segmented into a third sequence read fragment 36, and so forth until the end of the sequence read is reached. Each of these sequence read fragments can be considered a vector that is individually scored by the model 40 and used to look for contigs 42. In some embodiments, the segmentation segments a sequence read into sequence read fragments that are between 10 residues and 200 residues long, with a segmentation advance that is between 1 residues and 100 residues. In some such embodiments, each sequence read fragment is the same length. In some instances where there is insufficient sequence read to fill a sequence read fragment, the sequence read fragment is zero filled (e.g., the missing residues are scored as missing) so that all sequence read fragments are the same length. Alternatively, in other instances where there is insufficient sequence read to fill a sequence read fragment, the incomplete sequence read fragment is not used (discarded) so that all sequence read fragments are the same length. In some embodiments, there is only a single sequence read fragment 36 for each sequence read 34. In some embodiments, there are 2, 3, 4, 5, 6, 7, 8, 9, 10, 11, 12, 13, 14, 15 or more than 15 sequence read fragments 36 for a sequence read 34.

Block 2320. Referring to block 2320, in some embodiments, the encoding is one hot encoding. In other words, each element of the vector is assigned a value that represents a corresponding residue position in the sequence read. In such embodiments, each of the twenty naturally occurring residues is assigned a unique integer value (e.g., between 1 and 20) in a lookup table and this lookup table is used to convert residue names to the corresponding integer. Such one hot encoding is distinguished from other forms of encoding that consider not only the identify of a residue at a given residue position in the sequence read, but other properties as well, such as the identity of residues neighboring the residue or chemical properties of residues neighboring the residue.

Block 2322. Referring to block 2322, in some embodiments, the method includes assigning each respective sequence read in the plurality of sequence reads a corresponding scalar model score by inputting the vector, in the plurality of vectors, corresponding to the respective sequence read into a model (e.g., model 1924 of FIG. 1).

In some embodiments, the model 40 is a convolutional neural network. In some embodiments the model 40 is a neural network is composed of one 1D-convolutional layer and three fully connected layers, one of which is the final output layer.

In some embodiments, the model 40 is a neural network (e.g., a convolutional neural network and/or a residual neural network). Neural network algorithms, also known as artificial neural networks (ANNs), include convolutional and/or residual neural network algorithms (deep learning algorithms). Neural networks can be machine learning algorithms that may be trained to map an input data set to an output data set, where the neural network includes an interconnected group of nodes organized into multiple layers of nodes. For example, the neural network architecture may include at least an input layer, one or more hidden layers, and an output layer. The neural network may include any total number of layers, and any number of hidden layers, where the hidden layers function as trainable feature extractors that allow mapping of a set of input data to an output value or set of output values. As used herein, a deep learning algorithm (DNN) can be a neural network including a plurality of hidden layers, e.g., two or more hidden layers. Each layer of the neural network can include a number of nodes (or “neurons”). A node can receive input that comes either directly from the input data or the output of nodes in previous layers, and perform a specific operation, e.g., a summation operation. In some embodiments, a connection from an input to a node is associated with a parameter (e.g., a weight and/or weighting factor). In some embodiments, the node may sum up the products of all pairs of inputs, xi, and their associated parameters. In some embodiments, the weighted sum is offset with a bias, b. In some embodiments, the output of a node or neuron may be gated using a threshold or activation function, f, which may be a linear or non-linear function. The activation function may be, for example, a rectified linear unit (ReLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

The weighting factors, bias values, and threshold values, or other computational parameters of the neural network, may be “taught” or “learned” in a training phase using one or more sets of training data. For example, the parameters may be trained using the input data from a training data set and a gradient descent or backward propagation method so that the output value(s) that the ANN computes are consistent with the examples included in the training data set. The parameters may be obtained from a back propagation neural network training process.

Any of a variety of neural networks may be suitable for use in performing the methods disclosed herein. Examples can include, but are not limited to, feedforward neural networks, radial basis function networks, recurrent neural networks, residual neural networks, convolutional neural networks, residual convolutional neural networks, and the like, or any combination thereof. In some embodiments, the machine learning makes use of a pre-trained and/or transfer-learned ANN or deep learning architecture. Convolutional and/or residual neural networks can be used for analyzing an image of a subject in accordance with the present disclosure.

For instance, a deep neural network model includes an input layer, a plurality of individually parameterized (e.g., weighted) convolutional layers, and an output scorer. The parameters (e.g., weights) of each of the convolutional layers as well as the input layer contribute to the plurality of parameters (e.g., weights) associated with the deep neural network model. In some embodiments, at least 100 parameters, at least 1000 parameters, at least 2000 parameters or at least 5000 parameters are associated with the deep neural network model. As such, deep neural network models require a computer to be used because they cannot be mentally solved. In other words, given an input to the model, the model output needs to be determined using a computer rather than mentally in such embodiments. See, for example, Krizhevsky et al., 2012, “Imagenet classification with deep convolutional neural networks,” in Advances in Neural Information Processing Systems 2, Pereira, Burges, Bottou, Weinberger, eds., pp. 1097-1105, Curran Associates, Inc.; Zeiler, 2012 “ADADELTA: an adaptive learning rate method,” CoRR, vol. abs/1212.5701; and Rumelhart et al., 1988, “Neurocomputing: Foundations of research,” ch. Learning Representations by Back-propagating Errors, pp. 696-699, Cambridge, MA, USA: MIT Press, each of which is hereby incorporated by reference in its entirety for all purposes.

Neural network algorithms, including convolutional neural network algorithms, suitable for use as models 40 are disclosed in, for example, Vincent et al., 2010, “Stacked denoising autoencoders: Learning useful representations in a deep network with a local denoising criterion,” J Mach Learn Res 11, pp. 3371-3408; Larochelle et al., 2009, “Exploring strategies for training deep neural networks,” J Mach Learn Res 10, pp. 1-40; and Hassoun, 1995, Fundamentals of Artificial Neural Networks, Massachusetts Institute of Technology, each of which is hereby incorporated by reference. Additional example neural networks suitable for use as models are disclosed in Duda et al., 2001, Pattern Classification, Second Edition, John Wiley & Sons, Inc., New York; and Hastie et al., 2001, The Elements of Statistical Learning, Springer-Verlag, New York, each of which is hereby incorporated by reference in its entirety for all purposes. Additional example neural networks suitable for use as models are also described in Draghici, 2003, Data Analysis Tools for DNA Microarrays, Chapman & Hall/CRC; and Mount, 2001, Bioinformatics: sequence and genome analysis, Cold Spring Harbor Laboratory Press, Cold Spring Harbor, New York, each of which is hereby incorporated by reference in its entirety for all purposes.

In some embodiments the model 40 comprises one or more convolutional layers, where the respective parameters (e.g., weights) for each convolutional layer are filters. Each filter has a corresponding height and width. In typical embodiments, a respective filter is smaller than the input image to the corresponding convolutional layer.

In some embodiments, the model 40 comprises one or more pooling layers (e.g., downsampling layers) that are used to reduce the number of parameters (e.g., to reduce the computational complexity). In some embodiments, a pooling layer is interspersed between two other layers (e.g., two convolutional layers).

In some embodiments, a respective stride for a corresponding convolutional or pooling layer is at least 1, at least 2, at least 3, at least 4, at least 5, at least 6, at least 7, at least 8, at least 9, or at least 10. In some embodiments, the respective stride for a corresponding convolutional or polling layer is at most 10, 9, 8, 7, 6, 5, 4, 3, 2, or 1.

In some embodiments, a respective size for a corresponding convolutional or pooling layer is a respective matrix (n×n) of pixels. In some embodiments, n is at least 1, at least 2, at least 3, at least 4, at least 5, at least 6, at least 7, at least 8, at least 9, or at least 10. In some embodiments, n is at most 10, 9, 8, 7, 6, 5, 4, 3, 2, or 1. In some embodiments, a size of a corresponding pooling layer is smaller (e.g., has a smaller n) than the size of an upstream convolutional or pooling layer.

In some embodiments, the model 40 comprises one or more fully connected embedding layers that each comprises a corresponding set of weights. In some embodiments, a respective fully connected embedding layer directly or indirectly receives an output of the input layer. In some embodiments, an output of the respective embedding layer comprises a second number of dimensions different from the first number of dimensions. That is, in some embodiments, the second number of dimensions is less than the first number of dimensions.

In some embodiments, the model 40 comprises at least one hidden layer. Hidden layers are located between input and output layers (e.g., to capture additional complexity). In some embodiments, where there is a plurality of hidden layers, each hidden layer may have a same respective number of neurons.

In some embodiments, the model 40 comprises one or more classifying layers (e.g., output layers). In some embodiments, a classifying layer provides binary output (e.g., decides between two options such as “is a parasitic sequence” and “is not a parasitic sequence” or “is a viral sequence” or “is not a viral sequence”).

In some embodiments, the model 40 comprises a corresponding plurality of inputs, where each input in the corresponding plurality of inputs is for a one hot-encoded value of a corresponding residue in the sequence read fragment 36, a corresponding first hidden layer comprising a corresponding plurality of hidden neurons, where each hidden neuron in the corresponding plurality of hidden neurons (i) is fully connected to each input in the plurality of inputs, (ii) is associated with a first activation function type, and (iii) is associated with a corresponding weight in a plurality of weights for the model 40, and one or more corresponding neural network outputs, where each respective neural network output in the corresponding one or more neural network outputs (i) directly or indirectly receives, as input, an output of each hidden neuron in the corresponding plurality of hidden neurons, and (ii) is associated with a second activation function type. In some such embodiments, the model 40 is a fully connected network. In some such embodiments, the first activation function type and the second activation function type are the same or different and are each one of tanh, sigmoid, softmax, Gaussian, Boltzmann-weighted averaging, absolute value, linear, rectified linear unit (ReLU), bounded rectified linear, soft rectified linear, parameterized rectified linear, average, max, min, sign, square, square root, multiquadric, inverse quadratic, inverse multiquadric, polyharmonic spline, or thin-plate spline. In some embodiments, the model 40 is trained using a regularization on the corresponding weight of each hidden neuron in the plurality of hidden neurons. In some embodiments, the regularization includes an L1 or L2 penalty.

Block 2324. Referring to block 2324, in some embodiments, the model 40 includes a one-dimensional convolutional layer followed by one or more fully connected layers.

Block 2326. Referring to block 2326, in some embodiments, the model 40 assigns the corresponding model score using an activation function in the final fully connected layer in the one or more fully connected layers.

Block 2328. Referring to block 2328, in some embodiments, the activation function is a sigmoid activation function. In some embodiments, the activation function is a rectified linear unit (RcLU) activation function, a Leaky ReLU activation function, or other function such as a saturating hyperbolic tangent, identity, binary step, logistic, arcTan, softsign, parametric rectified linear unit, exponential linear unit, softPlus, bent identity, softExponential, Sinusoid, Sine, Gaussian, or sigmoid function, or any combination thereof.

Block 2330. Referring to block 2330, in some embodiments, the one or more fully connected layers is three or more fully connected layers. In some embodiments, the one or more fully connected layers is 4, 5, 6, 7, 8, 9, 10, or more fully connected layers.

Blocks 2332 and 2334. Referring to block 2332, in some embodiments, each corresponding scalar model score is a value between zero and one and a sequence read in the plurality of sequence reads satisfies the first threshold score when it has a model value that is between 0.60 and 1.0. Referring to block 2334 of FIG. 2C, in some embodiments, each corresponding scalar model score is a value between zero and one and a sequence read in the plurality of sequence reads satisfies the first threshold score when it has a value that is between 0.70 and 1.0. For instance, in some embodiments, each corresponding scalar model score is a value between zero and one and a sequence read in the plurality of sequence reads satisfies the first threshold score when it has a value that is between 0.60 and 1.0, between 0.65 and 1.0, between 0.70 and 1.0, between 0.75 and 1.0, between 0.80 and 1.0, between 0.85 and 1.0, between 0.90 and 1.0, or between 0.95 and 1.0.

In some embodiments, each corresponding scalar model score is a value between zero and one and a sequence read in the plurality of sequence reads satisfies the first threshold score when it has a value of at least 0.60, at least 0.65, at least 0.70, at least 0.75, at least 0.80, at least 0.85, at least 0.90, at least 0.95, or at least 1.0. In some embodiments, each corresponding scalar model score is a value between zero and one and a sequence read in the plurality of sequence reads satisfies the first threshold score when it has a value of at most 0.60, at most 0.65, at most 0.70, at most 0.75, at most 0.80, at most 0.85, at most 0.90, at most 0.95, or at most 1.0.

Blocks 2336-2338. Referring to blocks 2336-2338, in some embodiments, the model 40 includes 1,000 or more weights that are evaluated by the model during the assigning for each respective sequence read in the plurality of sequence reads. For instance, in some embodiments, the model includes 1,000 or more weights, 1,500 or more weights, 2,000 or more weights, 2,500 or more weights, 3,000 or more weights, 3,500 or more weights, 4,000 or more weights, 4,500 or more weights, 5,000 or more weights, 5,500 or more weights, 6,000 or more weights, 6,500 or more weights, 7,000 or more weights, 7,500 or more weights, 8,000 or more weights, 8,500 or more weights, 9,000 or more weights, 9,500 or more weights, 10,000 or more weights 10,500 or more weights, or 11,000 or more weights that are evaluated by the model during the assigning for each respective sequence read in the plurality of sequence reads.

In some embodiments, the model 40 includes at least 1,000 weights, at least 1,500 weights, at least 2,000 weights, at least 2,500 weights, at least 3,000 weights, at least 3,500 weights, at least 4,000 weights, at least 4,500 weights, at least 5,000 weights, at least 5,500 weights, at least 6,000 weights, at least 6,500 weights, at least 7,000 weights, at least 7,500 weights, at least 8,000 weights, at least 8,500 weights, at least 9,000 weights, at least 9,500 weights, at least 10,000 weights, at least 10,500 weights, at least 11,000 weights, that are evaluated by the model during the assigning for each respective sequence read in the plurality of sequence reads. In some embodiments, the model includes at most 1,000 weights, at most 1,500 weights, at most 2,000 weights, at most 2,500 weights, at most 3,000 weights, at most 3,500 weights, at most 4,000 weights, at most 4,500 weights, at most 5,000 weights, at most 5,500 weights, at most 6,000 weights, at most 6,500 weights, at most 7,000 weights, at most 7,500 weights, at most 8,000 weights, at most 8,500 weights, at most 9,000 weights, at most 9,500 weights, at most 10,000 weights, at most 10,500 weights, at most 11,000 weights, that are evaluated by the model during the assigning for each respective sequence read in the plurality of sequence reads.

Block 2340. Referring to block 2340, in some embodiments, the method includes selecting a subset of the plurality of sequence reads as a plurality of contig seeds. Each sequence read in the subset of sequence reads has a corresponding scalar model score that satisfies a first threshold score. In some embodiments the first threshold score is one that selects the 30 percent or less, 28 percent or less, 26 percent or less, 24 percent or less, 22 percent or less, 20 percent or less, 18 percent or less, 16 percent or less, 14 percent or less, 12 percent or less, 10 percent or less, 8 percent or less, 6 percent or less, 4 percent or less, 2 percent or less, 1 percent or less, or 0.5 percent or less of the initial plurality of sequence reads, scored by the model, that receive top model scores. In some alternative embodiments, the first threshold score is a threshold model score on a scale between 0 and 1, such as 0.30, 0.35, 0.40, 0.45, 0.50, 0.55, 0.60, 0.65, 0.70, 0.75, 0.80, 0.85, 0.90, 0.95, or 0.98.

Block 2342 and 2344. Referring to block 2342 of FIG. 2D, in some embodiments, the method includes aligning sequence reads in the plurality of sequence reads to the plurality of contig seeds through common k-mer sequences thereby forming a plurality of contigs. Referring to block 2344, in some embodiments, the aligning to a respective contig seed in the plurality of contig seeds terminates when an average scalar model score for sequence reads aligning to the respective contig seed fails to satisfy a second threshold score. For instance, in some embodiments, viral contigs are assembled using iterative search for substrings with exact matches between k-mers. In some embodiments these are 24 base pair k-mers. In some embodiments these are 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, or 42 base pair k-mers. Each seed is complemented from the left and right ends using its left-most and right-most k-mers. For both the left and right assembly, reads containing the left or right most k-mers in a different position from the read that is being searched are identified. The sequence read adding the maximal number of bases to the assembled contig is used to complement the left and right contigs. The model scores that were assigned to sequence reads (or to sequence read fragments) that are used to assemble each contig are averaged, and the assembly terminates if the average score is below a second threshold value (e.g., such as 0.5). Finally, the right and left contigs are concatenated, to yield a complete viral contig.

Block 2346. Referring to block 2346, in some embodiments, each corresponding scalar model score is a value between zero and one and the average scalar model score for sequence reads aligning to the respective seed fails to satisfy the second threshold score when the average scalar model score is less than 0.60. In some embodiments, each corresponding scalar model score is a value between zero and one and the average scalar model score for sequence reads aligning to the respective seed fails to satisfy the second threshold score when the average scalar model score is less than 0.50, 0.55, 0.60, 0.65, 0.70, 0.75, 0.80, 0.85, or 0.90.

Block 2348. Referring to block 2348, in some embodiments, each corresponding scalar model score is a value between zero and one and the average scalar model score for sequence reads aligning to the respective seed fails to satisfy the second threshold score when the average scalar model score is less than 0.50. For instance, some embodiments, each corresponding scalar model score is a value between zero and one and the average scalar model score for sequence reads aligning to the respective seed fails to satisfy the second threshold score when the average scalar model score is less than 0.60, less than 0.55, less than 0.50, less than 0.45, less than 0.40, less than 0.35, less than 0.30, less than 0.25, less than 0.20, or less than 0.15.

Block 2350. Referring to block 2350, in some embodiments, the common k-mer sequences have a length of 24 base pairs.

Block 2352. Referring to block 2352, in some embodiments, the common k-mer sequences have a common base pair length. In some embodiments, the common base pair length is an integer between 12 and 45. For instance, in some embodiments, the common base pair length is an integer between 12 and 45, between 12 and 40, between 12 and 35, between 12 and 30, between 12 and 25, between 12 and 20, between 12 and 15, between 17 and 45, between 17 and 40, between 17 and 35, between 17 and 30, between 17 and 25, between 17 and 20, between 22 and 45, between 22 and 40, between 22 and 35, between 22 and 30, between 22 and 25, between 27 and 45, between 27 and 40, between 27 and 35, between 27 and 30, between 32 and 45, between 32 and 40, between 32 and 35, between 37 and 45, between 37 and 40, or between 42 and 45. In some embodiments, the common base pair length is an integer of at least 12, at least 15, at least 17, at least 20, at least 22, at least 25, at least 27, at least 30, at least 32, at least 35, at least 37, at least 40, at least 42, or at least 45. In some embodiments, the common base pair length is an integer of at most 12, at most 15, at most 17, at most 20, at most 22, at most 25, at most 27, at most 30, at most 32, at most 35, at most 37, at most 40, at most 42, or at most 45.

Block 2354. Referring to block 2354, in some embodiments, the method includes using the plurality of contigs to identify one or more viral sequences in the subject. For instance, in some embodiments the contigs yielded by the assembly component ac used as inputs to blastn. Scc Altschul et al., 1997, “Gapped BLAST and PSI-BLAST: a new generation of protein database search programs,” Nucleic Acids Res 25, 3389-3402, which is hereby incorporated by reference. In some embodiments blastn is used to search each contig 44 against the RefSeq reference human viruses database, National Center for Biotechnology Information (NCBI), see Sayers et al., “Database resources of the National Center for Biotechnology Information,” Nucleic Acids Res 49, D10-d17 which is hereby incorporated by reference in its entirety, to which was added human papillomaviruses strains that are not in RefSeq from PAVE (see Van Doorslaer et al., 2017, “The Papillomavirus Episteme: a major update to the papillomavirus sequence database,” Nucleic Acids Res 45, D499-d506. In some such embodiments, reference viruses were searched using blastn, with default parameters except for a word size of 15 (lower than the default of 28), which was chosen to allow identification from short contigs.

In some embodiments blastn is used to search each contig 44 against more divergent viruses obtained from RVDB75 (https://hive.biochemistry.gwu.edu/rvdb/) which was then filtered to remove non-viral elements, endogenous viruses, and accessions that were consistently not verified using blastn against the nonredundant (nr) blast nucleotide database.

In some embodiments blastn is used to search each contig 44 against human endogenous viruses. For instance, in one embodiment a database of potentially functional HERVs was curated through evaluation of viral protein completeness (in contrast to a previous study that evaluated HERV expression in distinct RNAseq datasets as disclosed in Tokuyama et al., 2018, “ERVmap analysis reveals genome-wide transcription of human endogenous retroviruses,” Proc Natl Acad Sci USA 115, 12565-12572). The initial genomic locations of reported HERV elements were downloaded from the HER Vd HERV annotation database (https://herv.img.cas.cz) disclosed in Paces et al., 2004, “HERVd: the Human Endogenous Retro Viruses Database: update, Nucleic Acids Res 32, D50, doi: 10.1093/nar/gkh075 (2004), which is hereby incorporated by reference. The nucleotide sequences in hg19 for each reported HERV were extracted using twoBitToFa (See Karolchik et al., “The UCSC Table Browser data retrieval tool,” Nucleic Acids Res 32, D493-496. Then applied blastx is applied against NR with E-value cutoff of 1E-4, as well as a profile search (Yutin et al., 2012, “Phylogenomics of prokaryotic ribosomal proteins,” PLOS One 7, e36972, doi: 10.1371/journal.pone.0036972) against collected POL proteins, where the profile was obtained by collecting POL genes annotated in GenBank in lentiviruses (as of September 2016) and aligning their amino acid sequences using MAFFT (Katoh Standley, 2013, “MAFFT multiple sequence alignment software version 7: improvements in performance and usability,” Mol Biol Evol 30, 772-780, doi: 10.1093/molbev/mst010.

In some embodiments, the method includes using the plurality of contigs to identify one or more pathogenic sequences in the subject. In some embodiments such pathogenic sequences are those of a microorganism, such as a bacteria, fungus, protozoa (e.g., protozoan parasites), virus (e.g., DNA viruses and/or RNA viruses), algae, archaea, phage, or helminth (e.g., multicellular eukaryotic parasites). In some embodiments, when such sequences from a microorganism are discovered, the method further includes treating the subject for an infection (induced by the presence of the detected microorganism in the subject).

In some embodiments the discovered microorganism is a bacteria and, upon its discovery, the systems and methods further comprise treating the subject with an anti-bacterial composition to treat a bacterial infection in the subject.

In some embodiments the discovered microorganism is a virus and, upon its discovery, the systems and methods further comprise treating the subject with an anti-viral composition to treat a viral infection in the subject.

In some embodiments the discovered microorganism is a protozoa and, upon its discovery, the systems and methods further comprise treating the subject with an anti-protozoa composition to treat a protozoa infection in the subject.

In some embodiments the discovered microorganism is a fungus and, upon its discovery, the systems and methods further comprise treating the subject with an anti-fungal composition to treat a fungal infection in the subject.

In some embodiments the discovered microorganism is algae and, upon its discovery, the systems and methods further comprise treating the subject with an anti-algae composition to treat an algae infection (e.g., Protothecosis,) in the subject.

In any of the above embodiments the treatment is a composition comprising one or more active ingredients and one or more excipient and/or one or more pharmaceutically acceptable carrier and/or one or more diluent. These include all conventional solvents, dispersion media, fillers, solid carriers, coatings, antifungal and antibacterial agents, dermal penetration agents, surfactants, isotonic and absorption agents and the like. It will be understood that the compositions of the invention may also include other supplementary physiologically active agents.

An exemplary carrier is pharmaceutically “acceptable” in the sense of being compatible with the other ingredients of the composition (e.g., the one or more active ingredients) and not injurious to the patient. The compositions may conveniently be presented in unit dosage form and may be prepared by any methods well known in the art of pharmacy. Such methods include the step of bringing into association the one or more active ingredients with the carrier that constitutes one or more accessory ingredients. In general, the compositions are prepared by uniformly and intimately bringing into association the one or more active ingredients with liquid carriers or finely divided solid carriers or both, and then if necessary shaping the product.

Exemplary compounds, compositions or combinations of the present disclosure (e.g., the composition comprising the one or more active ingredients) formulated for intravenous, intramuscular or intraperitoneal administration, and may be administered by injection or infusion.

Injectables for such use can be prepared in conventional forms, either as a liquid solution or suspension or in a solid form suitable for preparation as a solution or suspension in a liquid prior to injection, or as an emulsion. Carriers can include, for example, water, saline (e.g., normal saline (NS), phosphate-buffered saline (PBS), balanced saline solution (BSS)), sodium lactate Ringer's solution, dextrose, glycerol, ethanol, and the like; and if desired, minor amounts of auxiliary substances, such as wetting or emulsifying agents, buffers, and the like can be added. Proper fluidity can be maintained, for example, by using a coating such as lecithin, by maintaining the required particle size in the case of dispersion and by using surfactants.

The compound, composition or combinations of the present disclosure (e.g., the one or more active ingredients) may also be suitable for oral administration and may be presented as discrete units such as capsules, sachets or tablets each containing a predetermined amount of the one or more active ingredients; as a powder or granules; as a solution or a suspension in an aqueous or non-aqueous liquid; or as an oil-in-water liquid emulsion or a water-in-oil liquid emulsion. The one or more active ingredients may also be presented as a bolus, electuary or paste.

A tablet may be made by compression or molding, optionally with one or more accessory ingredients. Compressed tablets may be prepared by compressing in a suitable machine the one or more active ingredients (e.g., the composition comprising the modified polymer) in a free-flowing form such as a powder or granules, optionally mixed with a binder (e.g inert diluent, preservative disintegrant (e.g. sodium starch glycolate, cross-linked polyvinyl pyrrolidone, cross-linked sodium carboxymethyl cellulose) surface-active or dispersing agent. Molded tablets may be made by molding in a suitable machine a mixture of the powdered compound moistened with an inert liquid diluent. The tablets may optionally be coated or scored and may be formulated so as to provide slow or controlled release of the one or more active ingredients therein using, for example, hydroxypropylmethyl cellulose in varying proportions to provide the desired release profile. Tablets may optionally be provided with an enteric coating, to provide release in parts of the gut other than the stomach.

The compound, composition or combinations of the present disclosure (e.g., the one or more active ingredients) may be suitable for topical administration in the mouth including lozenges comprising the one or more active ingredients in a flavored base, usually sucrose and acacia or tragacanth gum; pastilles comprising the one or more active ingredients in an inert basis such as gelatine and glycerin, or sucrose and acacia gum; and mouthwashes comprising the one or more active ingredients in a suitable liquid carrier.

The compound, composition or combinations of the present disclosure (e.g., the one or more active ingredients) may be suitable for topical administration to the skin may comprise the compounds dissolved or suspended in any suitable carrier or base and may be in the form of lotions, gel, creams, pastes, ointments and the like. Suitable carriers include mineral oil, propylene glycol, polyoxyethylene, polyoxypropylene, emulsifying wax, sorbitan monostearate, polysorbate 60, cetyl esters wax, cetearyl alcohol, 2-octyldodecanol, benzyl alcohol and water. Transdermal patches may also be used to administer the compounds of the present disclosure.

The compound, composition or combination of the present disclosure (e.g., the one or more active ingredients) may be suitable for parenteral administration include aqueous and non-aqueous isotonic sterile injection solutions which may contain anti-oxidants, buffers, bactericides and solutes which render the compound, composition or combination isotonic with the blood of the intended recipient; and aqueous and non-aqueous sterile suspensions which may include suspending agents and thickening agents. The compound, composition or combination may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules and tablets of the kind previously described.

It should be understood that in addition to the one or more active ingredients particularly mentioned above, the composition or combination of this present disclosure (e.g., the one or more active ingredients) may include other agents conventional in the art having regard to the type of composition or combination in question, for example, those suitable for oral administration may include such further agents as binders, sweeteners, thickeners, flavoring agents disintegrating agents, coating agents, preservatives, lubricants and/or time delay agents. Suitable sweeteners include sucrose, lactose, glucose, aspartame or saccharine. Suitable disintegrating agents include cornstarch, methylcellulose, polyvinylpyrrolidone, xanthan gum, bentonite, alginic acid or agar. Suitable flavoring agents include peppermint oil, oil of wintergreen, cherry, orange or raspberry flavoring. Suitable coating agents include polymers or copolymers of acrylic acid and/or methacrylic acid and/or their esters, waxes, fatty alcohols, zein, shellac or gluten. Suitable preservatives include sodium benzoate, vitamin E, alpha-tocopherol, ascorbic acid, methyl paraben, propyl paraben or sodium bisulphite. Suitable lubricants include magnesium stearate, stearic acid, sodium oleate, sodium chloride or talc. Suitable time delay agents include glyceryl monostearate or glyceryl distearate.

FIG. 3 illustrates a flow chart of methods (e.g., method 3000) for identifying a cancer, in accordance with embodiments of the present disclosure. Specifically, an exemplary method 3000 for determining a prognosis of a subject surviving a cancer is provided, in accordance with some embodiments of the present disclosure. In the flow charts, the preferred parts of the methods are shown in solid line boxes, whereas optional variants of the methods, or optional equipment used by the methods, are shown in dashed line boxes. As such, FIG. 3 illustrates methods for determining a prognosis of a subject surviving a cancer.

Various modules in the memory 112 of the computer system 100 perform certain processes of the methods described in FIG. 3, unless expressly stated otherwise. Furthermore, it will be appreciated that the processes in FIG. 3 can be encoded in a single module or any combination of modules.

Block 3300. Referring to block 3300 of FIG. 3, a method for determining a prognosis of a subject surviving a cancer is provided.

Block 3302. Referring to block 3302, in some embodiments, the cancer is endometrial cancer.

Block 3304. Referring to block 3304, in some embodiments, the method includes using a computer system (e.g., computer system 100 of FIG. 1). The computer system includes one or more processing cores (e.g., CPU 102 of FIG. 1) and a memory (e.g., memory 112 of FIG. 1).

Block 3306. Referring to block 3306, in some embodiments, the method includes obtaining, in electronic form, a plurality of nucleic acid sequence reads from a biological sample obtained from the subject. The plurality of sequence reads is free of sequence reads associated with a reference genome of the species of the subject. For instance, in some embodiments, the plurality of nucleic acid sequence reads are mapped using a mapping program, such as Bowtie2 against hg19 (1000 Genomes version) and PhiX phage (NC_001422), and only the unmapped reads are kept in the plurality of nucleic acid sequence reads.

Block 3308. Referring to block 3308, in some embodiments, the biological sample is a tumor biopsy. In other embodiments, the biological sample is any of the samples disclosed herein.

Block 3310. Referring to block 3310, in some embodiments, the method includes determining whether sequence reads from an exogenous virus are present in the plurality of sequence reads. When sequence reads from an exogenous virus are present in the plurality of sequence reads, the method further includes up-weighting the prognosis that the subject will survive the cancer. When the plurality of sequence reads is free of sequences from an exogenous virus, the method further includes down-weighting the prognosis that the subject will survive the cancer.

Block 3312. Referring to block 3312, in some embodiments, the exogenous virus is an arthropod virus.

Block 3314. Referring to block 3314, in some embodiments, the arthropod virus is in the Betairidovirinae family.

Block 3316. Referring to block 3316, in some embodiments, the arthropod virus is Armadillidium vulgare iridescent virus.

FIG. 4 illustrates a flow chart of methods (e.g., method 4000) for identifying a cancer, in accordance with embodiments of the present disclosure. Specifically, an exemplary method 4000 for determining a prognosis of a subject surviving a cancer is provided, in accordance with some embodiments of the present disclosure. In the flow charts, the preferred parts of the methods are shown in solid line boxes, whereas optional variants of the methods, or optional equipment used by the methods, are shown in dashed line boxes. As such, FIG. 4 illustrates methods for determining a prognosis of a subject surviving a cancer.

Various modules in the memory 112 of the computer system 100 perform certain processes of the methods described in FIG. 4, unless expressly stated otherwise. Furthermore, it will be appreciated that the processes in FIG. 4 can be encoded in a single module or any combination of modules.

Block 4300. Referring to block 4300 of FIG. 4, a method for determining a prognosis of a subject surviving a cancer is provided.

Block 4302. Referring to block 4302, in some embodiments, the cancer is endometrial cancer.

Block 4304. Referring to block 4304, in some embodiments, the method includes using a computer system (e.g., computer system 100 of FIG. 1). The computer system includes one or more processing cores (e.g., CPU 102 of FIG. 1) and a memory (e.g., memory 112 of FIG. 1).

Block 4306. Referring to block 4306, in some embodiments, the method includes obtaining, in electronic form, a plurality of nucleic acid sequence reads from a biological sample obtained from the subject. The plurality of sequence reads is free of sequence reads associated with a reference genome of the species of the subject. For instance, in some embodiments, the plurality of nucleic acid sequence reads are mapped using a mapping program, such as Bowtie2 against hg19 (1000 Genomes version) and PhiX phage (NC_001422), and only the unmapped reads are kept in the plurality of nucleic acid sequence reads.

Block 4308. Referring to block 4308, in some embodiments, the method includes determining whether sequence reads encoding YP_009046765, YP_009046752 and YP_009046774 are present in the plurality of sequence reads. When the plurality of sequence reads indicate that YP_009046765, YP_009046752 or YP_009046774 is highly expressed in the biological sample, the method further includes down-weighting the prognosis of the subject surviving the cancer. When the plurality of sequence reads indicate that YP_009046765, YP_009046752 or YP_009046774 is not highly expressed in the biological sample, the method further includes up-weighting the prognosis of the subject surviving the cancer.

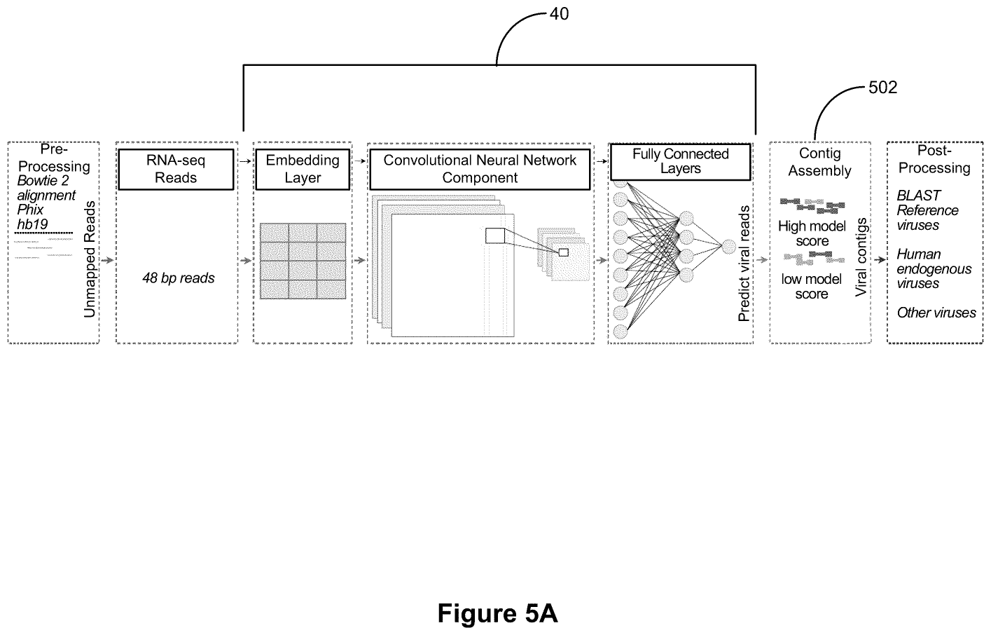

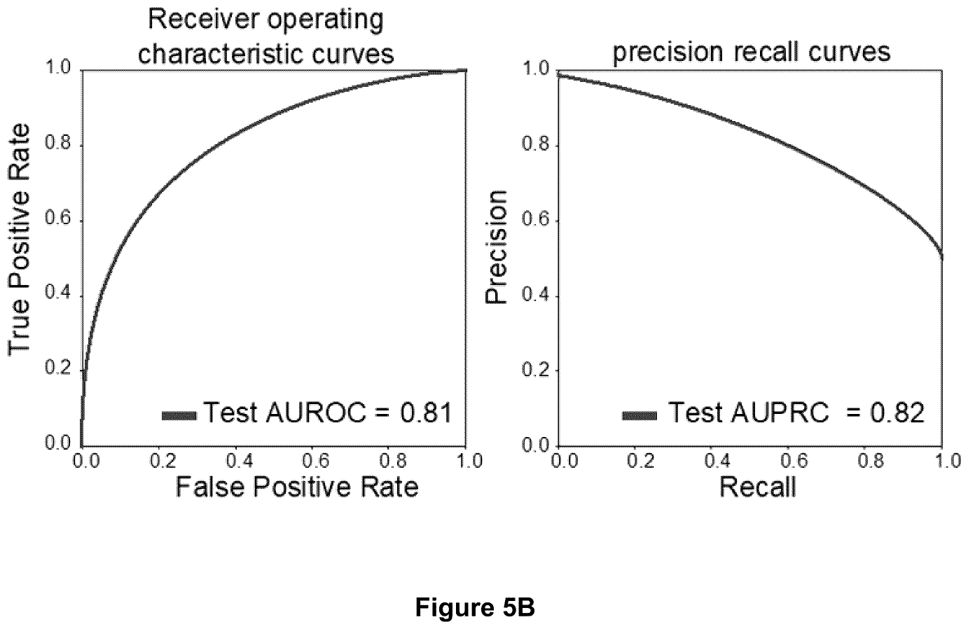

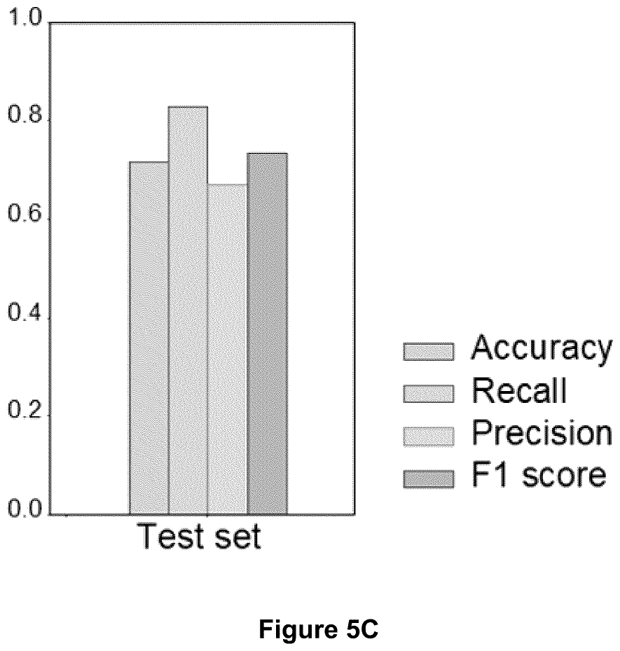

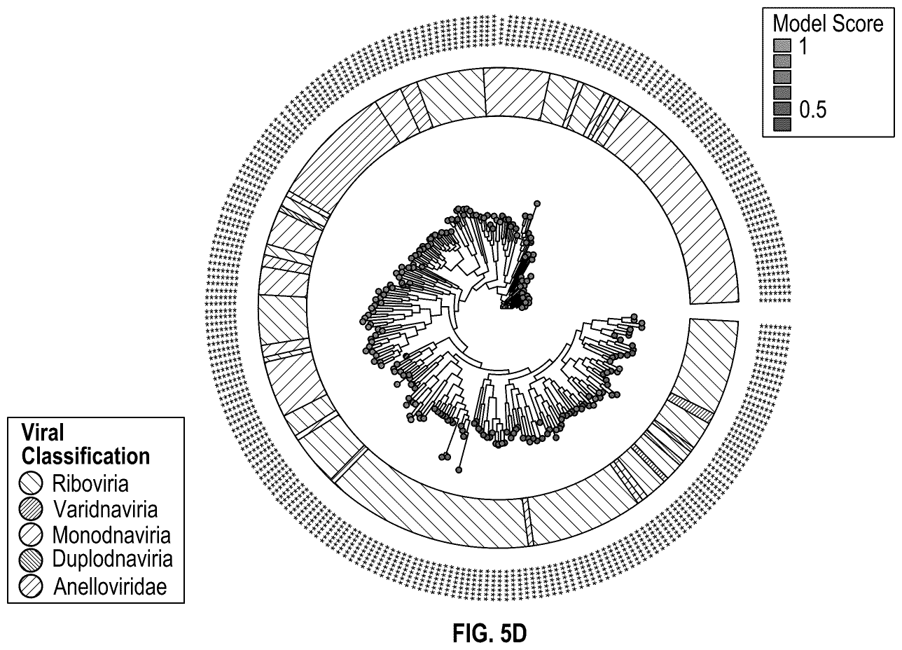

Referring to Table 1, Table 1 provides average model scores assigned to different human viruses plotted in FIG. 5D. Model scores were averaged across all 48 bp segments for each virus, using 2 bp window size.

Referring to Table 2, Table 2 provides TCGA sample information of 7272 samples from 14 cancer types used throughout the systems and methods of the present disclosure.

Referring to Table 3, Table 3 provides RefSeq viruses identified in 7272 TCGA samples from 14 cancer types, together with tumor mutation burden (TMB), chromosome level copy number alterations (CNA) overall survival time and death (0=survival, 1=death) for each TCGA sample considered by the present disclosure.

Referring to Table 4, Table 4 provides human endogenous viruses identified in 7272 TCGA samples from 14 cancer types. The ERV identifier can be mapped via Table 5 to the hg19 genomic interval that includes the ERV.

Referring to Table 5, Table 5 provides Intact retroviral genes, chromosomal location (hg19 assembly) and human gene and distance (measured as minimum between the number of bp from the start and end of each gene, where intronic HERVs are not distinguished) of each HERV to the nearest gene identified in TCGA tumor samples. The distance from the nearest SNP (dist_from_SNP) and the phenotype associated with the nearest SNP (SNP distance) are provided, and −1 values are assigned if no disease associated SNP was found located near a HERV.

Referring to Table 6, Table 6 provides Associations between HERV presence and tumor mutation burden or chromosomal aneuploidy across the 14 cancer types from TCGA. The values correspond to one sided Wilcoxon rank-sum p-values. TMB_greater and TCNA_greater test whether the TMB or CNA is greater in the presence of each HERV, and TMB_less and TCNA_less test whether the TMB or CNA is lower in the presence of each HERV.

Referring to Table 7, Table 7 provides Hyper-geometric enrichment p-values evaluating enrichment between somatic mutations in 10 frequently mutated cancer driver genes, and the expression of 36 HERVs that were found frequently expressed in cancer tissues.

Referring to Table 8, Table 8 provides Somatic mutations in frequently mutated cancer driver genes for cancer types in which HERV expression was associated with poor survival, and HERVs identified in TCGA samples within these cancer types.

Referring to Table 9, Table 9 provides Divergent unexpected viruses found expressed in 7272 samples from 14 cancer types from TCGA used throughout the present disclosure.

Referring to Table 10, Table 10 provides The IIV31 proteins identified in endometrial cancer (UCEC) samples with the tumor mutation burden and chromosomal ancuploidy scores. −1 values are assigned to samples with RNA sequencing data that did not have mutation or copy number information to evaluate the TMB or CNA.

### Example 1: Systems and Methods of the Present Disclosure

### Example 1.1: Training a Neural Network to Distinguish Viral RNA Sequencing Reads

A model 40 in accordance with this example was composed of two main components, illustrated in FIG. 5A. The first was a deep learning model 40, which was trained to accurately distinguish viral from human reads using RNA-sequencing. The second model 502 assembles the predicted viral reads into contigs. The trained neural network 40 was composed of one 1D-convolutional layer and three fully connected layers, one of which was the final output layer. The RNA sequences were one-hot encoded to vectors that were given as input to the model. The learning rate was set to 0.0005, in which the present disclosure used 64 filters with ReLU as an activation function in the convolutional layer, followed by one pooling layer for feature extraction. The global extracted features from the convolutional layer are passed to three fully connected layers, to make a prediction based on a sigmoid activation function in the output layer.

To train the model 40, the human and viral sequencing data was collected. Coding sequences of human and other placentals viruses downloaded from the Virus Variation Resource. Additional details and information is found at Hatcher et al., 2017, “Virus Variation Resource—improved response to emergent viral outbreaks,” Nucleic Acids Res (45), pg. D482-d490, which is hereby incorporated by reference in its entirety for all purposes. Human transcripts for hg19 were downloaded from NCBI Human Genome Resources. Additional details and information is found at Sayers et al., 2021, “Database resources of the National Center for Biotechnology Information,” Nucleic Acids Res (49), pg. D10-d17, which is hereby incorporated by reference in its entirety for all purposes. These sequences were segmented into 48 bp segments, which is the read length for the RNAseq in almost all tumor types in TCGA; only a few tumor types that were added chronologically last to TCGA used longer reads. This example utilized a 48 bp window size for human transcripts, and a 2 bp window size for viral sequences, to balance the positive and negative data. Then, these were randomly split (where all segments of each transcript were considered together) into balanced train, validation, and test sets (n=8,000,000, 800,000 and 2,558,044, respectively).

The performance of the model was evaluated using the Area Under the Receiver Operating Characteristic Curve (AUROC), the Area Under the Precision Recall Curve (AUPRC), as well as accuracy, precision, recall, and F1-score, for the test dataset. Multiple models were trained with different architectures and hyperparameters and then the model with highest average between the validation-set AUROC and recall was selected. The model was trained using TensorFlow 2.6.0 and Keras. Additional details and information is found at Chollet, F., 2015, “Keras,” Github, Github repository, github.com/fchollet/keras (accessed Oct. 19, 2022), which is hereby incorporated by reference in its entirety for all purposes.

### Example 1.2: Assembling Viral Contigs from Neural Network Predicted Viral Reads

Once the model predicted the probability of a viral origin of each read, reads with model scores more than 0.7 were used as seeds to assemble viral contigs. Viral contigs were assembled using iterative search for substrings with exact matches between 24 bp k-mers. Each seed was complemented from the left and right ends using its left-most and right-most 24 bp k-mers. For both the left and right assembly, reads including the left or right most k-mers in a different position from the read that was searched were identified. The read adding the maximal number of bases to the assembled contig was used to complement the left and right contigs. The model scores that were assigned to reads that are used to assemble each contig were averaged, and the assembly was terminated when the average score was below 0.5. Finally, the right and left contigs were concatenated, to yield a complete viral contig. This example was implemented in Python 3 and subsequently in C, which improved the running time by more than an order of magnitude for inputs with large numbers of reads.

### Example 1.3: Data Pre-Processing

RNA-sequencing data from Genomic Data Commons (GDC; portal.gdc.cancer.gov/) in the form of BAM files was utilized in this example. Additional details and information is found at Grossman et al., 2016, “Toward a Shared Vision for Cancer Genomic Data,” N Engl J Med (375), pg. 1109-1112, which is hereby incorporated by reference in its entirety for all purposes. High quality reads were selected and mapped with Bowtie2 against hg19 (1000 Genomes version) and PhiX phage (NC_001422), and only the unmapped reads were kept. Then, paired end reads were merged and converted to fastq files, which were used as input for the model 40 in accordance with this example, to yield predicted viral contigs.

### Example 1.4: Viral Databases

Viral contigs yielded by Example 1.3 were used as inputs to blasn. Additional details and information is found at Altschul et al., 1997, “Gapped BLAST and PSI-BLAST: a new generation of protein database search programs,” Nucleic Acids Res (25), pg. 3389-3402, which is hereby incorporated by reference in its entirety for all purposes. Three databases were used to search for viruses (with E-value threshold of 0.01):
(1) RefSeq reference human viruses, downloaded from the National Center for Biotechnology Information (NCBI), to which the present disclosure added human papillomaviruses strains that are not in RefSeq from PAVE (pave.niaid.nih.gov). Additional details and information is found at Sayers et al., 2021; Van Doorslaer et al., 2017, “The Papillomavirus Episteme: a major update to the papillomavirus sequence database,” Nucleic Acids Res (45), pg. D499-d506, each of which is hereby incorporated by reference in its entirety for all purposes. Reference viruses were searched using blastn, with default parameters except for a word size of 15 (lower than the default of 28), which was chosen to allow identification from short contigs.
(2) more divergent viruses obtained from RVDB (hive.biochemistry.gwu.edu/rvdb/) which was then filtered to remove non-viral elements, endogenous viruses, and accessions that were consistently not verified using blastn against the nonredundant (nr) blast nucleotide database. Additional details and information is found at Goodacre et al., 2018, “A Reference Viral Database (RVDB) To Enhance Bioinformatics Analysis of High-Throughput Sequencing for Novel Virus Detection,” mSphere (3), pg. doi: 10.1128/mSphereDirect.00069-18, which is hereby incorporated by reference in its entirety for all purposes.
(3) Human endogenous viruses. The present disclosure curated a database of potentially functional HERVs through evaluation of viral protein completeness (in contrast to a previous study that evaluated HERV expression in distinct RNAseq datasets). Additional details and information is found at Tokuyama et al., 2018, “ERVmap analysis reveals genome-wide transcription of human endogenous retroviruses,” Proc Natl Acad Sci USA (115), pg. 12565-12572, doi: 10.1073/pnas. 1814589115, which is hereby incorporated by reference in its entirety for all purposes. The initial genomic locations of reported HERV elements were obtained from the HERVd HERV annotation database (herv.img.cas.cz). Additional details and information is found at Paces et al., 2004, “HERVd: the Human Endogenous Retro Viruses Database: update,” Nucleic Acids Res (32), pg. D50, which is hereby incorporated by reference in its entirety for all purposes. The nucleotide sequences in hg19 for each reported HERV were extracted using twoBitToFa. Additional details and information is found at Karolchik et al., 2004, “The UCSC Table Browser data retrieval tool,” Nucleic Acids Res (32), pg. D493-496, which is hereby incorporated by reference in its entirety for all purposes. The present disclosure then applied blastx against NR with E-value cutoff of 1E-4, as well as a profile search against collected POL proteins, where the profile was obtained by collecting POL genes annotated in GenBank in lentiviruses (as of September 2016) and aligning their amino acid sequences using MAFFT. Additional details and information is found at Yutin et al., 2012, “Phylogenomics of prokaryotic ribosomal proteins,” PLOS One (7), pg. e36972, doi: 10.1371/journal.pone.0036972; Katoh et al., 2013, “MAFFT multiple sequence alignment software version 7: improvements in performance and usability,” Mol Biol Evol (30), pg. 772-780, doi: 10.1093/molbev/mst010, each of which is hereby incorporated by reference in its entirety for all purposes. Sequences with at least one identified retroviral protein motif of: POL/RT, GAG or ENV were extracted, yielding 3,044 HERVs that were considered for search in TCGA samples (Table 5).

### Example 1.5: Analysis of Divergent Viruses

All instances of divergent viruses identified in TCGA samples were verified using blastn against nr, to support that the virus strain is indeed the best match to a viral contig generated by the disclosed methods. Non-reference viruses (divergent viruses and viruses of non-human hosts) that were identified and verified in more than one sample were additionally searched using the STAR aligner across tumor types where these viruses were identified through viRNAtrap. Additional details and information is found at Dobin et al. 2013, “STAR: ultrafast universal RNA-seq aligner,” Bioinformatics (29), pg. 15-21, which is hereby incorporated by reference in its entirety for all purposes. The following accessions were additionally searched using STAR to increase sample coverage (as these were the most interesting divergent strains found across multiple samples): Bermuda grass latent virus (NC_032405), Armadillidium vulgare iridescent virus IIV31 (NC_024451), Geobacillus virus (NC_009552), and the Human lung-associated vientovirus (NC_055523).

### Example 1.6: Genomic Correlates of Viral Expression

The present disclosure correlated viral expression with genomic markers across TCGA samples. Chromosomal aneuploidy levels for TCGA samples were extracted from and the total number of chromosome-arm-level alterations was used. Additional details and information is found at Taylor et al., 2018, “Genomic and Functional Approaches to Understanding Cancer Aneuploidy,” Cancer Cell (33), pg. 676-689.e673, which is hereby incorporated by reference in its entirety for all purposes. The tumor mutation burden was defined to be the total number of somatic mutations in each sample, downloaded from the Xena browser (xenabrowser.net). Additional details and information is found at Goldman et al., 2020, “Visualizing and interpreting cancer genomics data via the Xena platform,” Nat Biotechnol (38), pg. 675-678, which is hereby incorporated by reference in its entirety for all purposes. CIBERSORT software was applied to TCGA samples using the default set of 22 immune-cell signatures. See Newman et al., 2015.

### Example 1.7: Experimental Validation of the Geobacillus Virus E2 in Ovarian Cancer Cell Lines

Reverse-transcriptase qPCR (RT-qPCR) RNA was extracted using TRIzol reagent (Invitrogen, cat. no. 15596026). Extracted RNA was used for reverse-transcriptase PCR using a High-capacity cDNA reverse transcription kit (Thermo Fisher, cat. no. 4368814). Quantitative PCR was performed using a QuantStudio 3 real-time PCR system. GAPDH was used as an internal control. The fold change was calculated using the 2-AACt method.

### Example 1.8: Identification of Trichomonas vaginalis-Positive Samples

UCEC unmapped (to hg19) reads were aligned to the reference genome of Trichomonas vaginalis (GCF_000002825) strain G3 using blastn with E-value<1e-8 and more than 90% identity. Additional details and information is found at Altschul et al., 1997; Carlton et al., 2007, “Draft genome sequence of the sexually transmitted pathogen Trichomonas vaginalis,” Science (315), pg. 207-212, which is hereby incorporated by reference in its entirety for all purposes. These thresholds were set to remove false positives that were frequent when aligning against Trichomonas vaginalis when examining both blastn and STAR aligner. See Altschul et al., 1997; Dobin et al, 2013. TV reads for each TV-positive sample were verified by manual inspection of the output alignments.

### Example 1.9: Statical Methods

Survival analysis, including Kaplan Meier curves plots, log rank test and proportional hazards test p-values were obtained using the Python lifelines package (v0.26.4). Additional details and information is found at Davidson-Pilon, 2019, “Lifelines: survival analysis in Python,” Journal of Open Source Software (4), pg. 1317 which is hereby incorporated by reference in its entirety for all purposes. P-values comparing TMB and aneuploidy between two groups correspond were computed with two-sided Wilcoxon rank-sum tests. Heatmap clustograms were generated through seaborn clustermap.

### Example 2: The Model Framework

To identify viruses in the human transcriptome, the present disclosure first trained a neural network to distinguish viral reads based on short sequences. The present disclosure collected positive (viral) and negative (human) transcripts that were segmented into 48 bp fragments and divided into training and test sets (FIG. 5A, method 2000, method 3000, method 4000, etc.). The present disclosure used different metrics to evaluate the ability of the model to identify viral sequences based on short segments. The model yielded test-set performance: area under the receiver operating characteristic curve (AUROC) of 0.81 area under the precision recall curve (AUPRC) of 0.82 (FIG. 5B), an accuracy of 0.71, recall of 0.83, precision of 0.67, and F1-score of 0.74, for the validation and test sets, respectively (FIG. 5C). Examining the average model performance across segments from different human viruses, the present disclosure found that human single-stranded DNA viruses from taxon Monodnaviria were assigned with high confidence, whereas, for RNA viruses, the present disclosure observed more variation in model confidence. For example, the model confidently predicted the viral origin of sequences from Ebola and influenza viruses, but assigned borderline scores to sequences from several Phenuiviridae members such as Dabie bandavirus (FIG. 5D, Table 1).

Based on the trained neural network, a computational framework was built (FIG. 5A; method 2000 of FIGS. 2A through 2D, method 3000 of FIG. 3, method 4000 of FIG. 4, etc.) to identify viral contigs from tumor RNAseq and applied the framework to 7272 samples from 14 cancer types in The Cancer Genome Atlas (TCGA), from which 6717 were tumor samples and 555 were non-cancer samples matched to a cancer sample from the same individual (Table 2). Additional details and information is found at Weinstein et al., 2013, “The Cancer Genome Atlas Pan-Cancer analysis project,” Nat Genet (45), pg. 1113-1120, which is hereby incorporated by reference in its entirety for all purposes. In pre-processing, reads were extracted that were not aligned to the human genome (hg19) or to the phiX phage that was identified as a frequent contaminant. Additional details and information is found at Mukherjee et al., 2015, “Large-scale contamination of microbial isolate genomes by Illumina PhiX control,” Stand Genomic Sci (10), pg. 18, which is hereby incorporated by reference in its entirety for all purposes. The computational framework, an example of model 40, was then applied to unaligned RNA reads (to reduce the running time of computational framework), to detect viral reads and assemble predicted viral contigs. Finally, in post-processing analysis, the present disclosure used blastn to compare the assembled viral contigs to three curated viral databases. See Altschul et al., 1997. The present disclosure identified viral contigs originating from reference viruses that are expected in cancer tissues, human endogenous viruses, and candidate novel or more divergent viruses, which are expressed in different cancer types

### Example 3: Identifying Reference Tumor Viruses

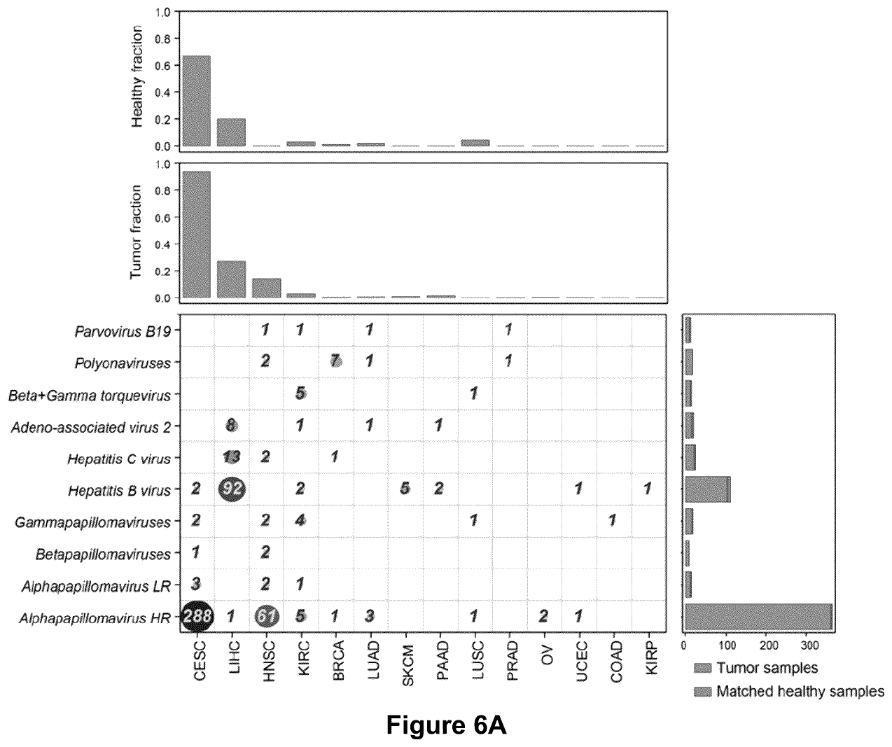

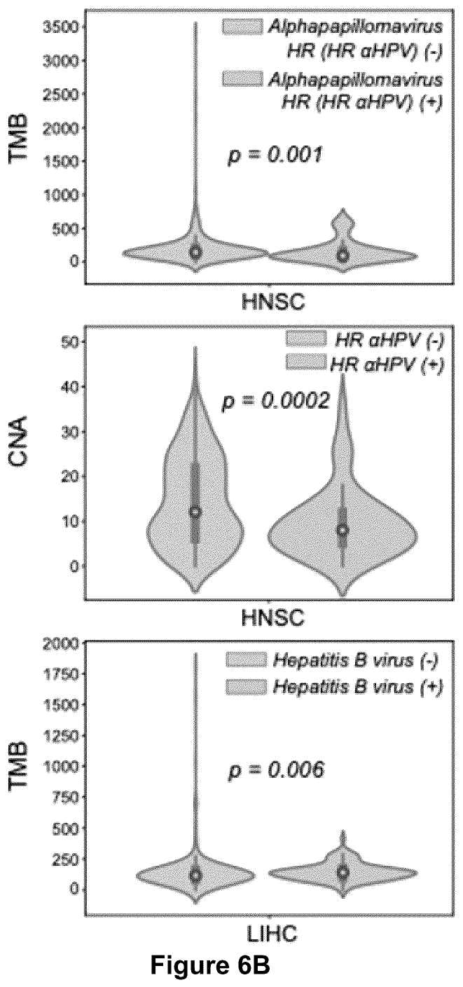

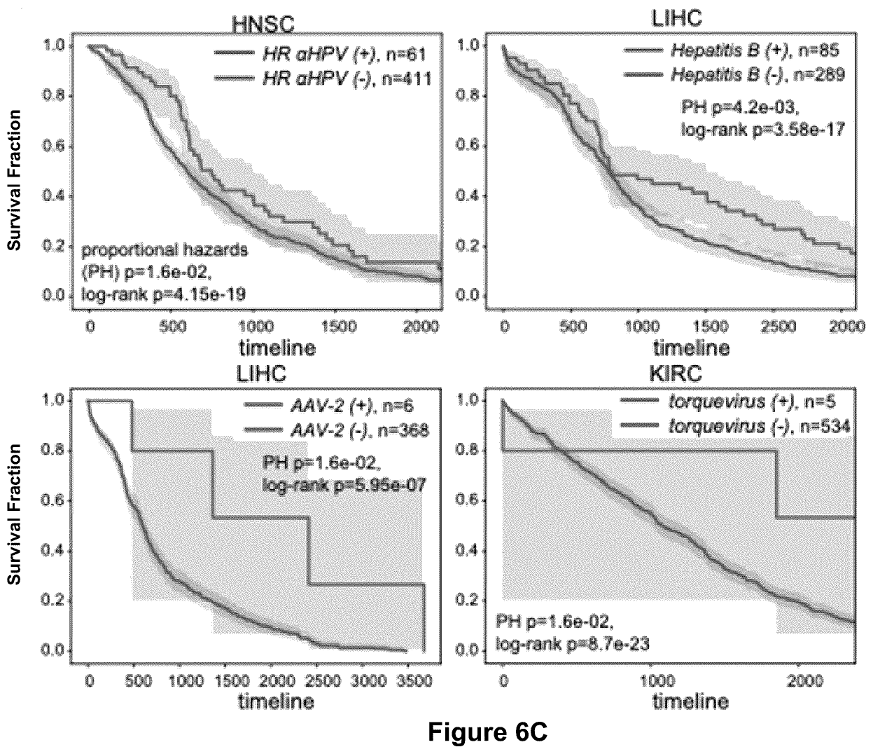

The present disclosure first characterized the presence of known cancer-associated human viruses in different tumor types. High-risk human Alphapapillomavirus strains (HR-αHPVs) were most frequently detected; the type observed in the majority of TCGA samples is HPV16. This is expected because HR-αHPVs, such as HPV16 and HPV18, underlie approximately 5% of cancer cases worldwide while low-risk human Alphapapillomavirus (LR-αHPV) strains, such as HPV54 and HPV201, are mostly associated with the development of genital warts but not cancer. Additional details and information is found at Coursey et al., 2021, “Regulation of Human Papillomavirus 18 Genome Replication, Establishment, and Persistence by Sequences in the Viral Upstream Regulatory Region,” J Virol (95), pg. e0068621; Doorbar et al., 2012, “The biology and life-cycle of human papillomaviruses,” Vaccine 30(5), pg. F55-70; each of which is hereby incorporated by reference in its entirety for all purposes. The present disclosure found at least one HR-αHPV in 288 CESC samples (286 squamous cell carcinoma samples and 2 non-cancer samples). The present disclosure found 61 HNSC samples, and a total of 14 samples across other cancer types, that include a contig from at least one HR-αHPV (FIG. 6A). LR-αHPVs were identified in a small set of samples mostly from matched non-cancer tissues, including cervix and head and neck (FIG. 6A, Table 3).

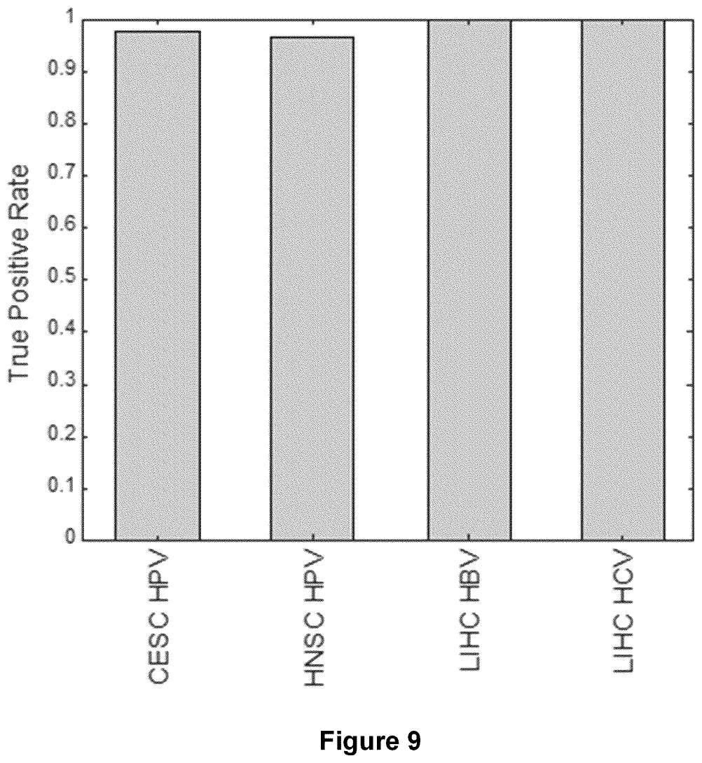

Hepatitis B virus (HBV) was the second most frequently detected virus across TCGA samples. HBV infections and Hepatitis C virus (HCV) infections are two primary causes of liver cancer and may co-occur in a patient. Additional details and information is found at Cantalupo et al., 2018, which is hereby incorporated by reference in its entirety for all purposes. The present disclosure determined HBV expression in 85 LIHC tumor samples and 7 non-cancer samples, and HCV in 13 LIHC tumor samples. HBV was also found in a few tumor samples and matched non-cancer samples from other cancer types (FIG. 6A). The present disclosure determined, by comparing samples predicted as virus-positive by viRNAtrap to the samples annotated as virus-positive in the TCGA clinical annotations, the present disclosure determined that the true positive of viRNAtrap were above 95% for HR-αHPVs (in CESC and HNSC), and for HCV and HBV in LIHC, supporting that viRNAtrap correctly identifies samples expressing known cancer viruses (FIG. 9). In addition, the present disclosure found adeno-associated virus 2 (AAV2) in 8 LIHC samples, 6 from tumors and 2 from non-cancer samples. AAV2 is a small DNA virus that has the potential to integrate into human genes and contribute to oncogenesis, although the current evidence is insufficient for AAV2 to be included in the consensus list of oncogenic viruses. Additional details and information is found at Schäffer et al., 2021, “Integration of adeno-associated virus (AAV) into the genomes of most Thai and Mongolian liver cancer patients does not induce oncogenesis,” BMC Genomics (22), pg. 814; Bayard et al., 2018, “Cyclin A2/E1 activation defines a hepatocellular carcinoma subclass with a rearrangement signature of replication stress,” Nat Commun (9), pg. 5235, each of which is hereby incorporated by reference in its entirety for all purposes. A recent study that addressed discrepancies in AAV2 expression across TCGA samples found at least one AAV2 read in 11 LIHC samples. However, in three of these samples only one AAV2 read was found, which is not sufficient for detection with the viRNAtrap pipeline. Notably, previous studies that systematically characterized viral presence across TCGA did not identify AAV2 in more than six LIHC samples, demonstrating the sensitivity of viRNAtrap compared to other computational methods. Additional details and information is found at Cantalupo et al., 2018; Shaffer et al., 2021. The present disclosure additionally detected AAV2 in one KIRC sample, one PAAD sample and one matched non-cancer sample from LUAD (FIG. 6A).

The present disclosure found several samples that express human polyomaviruses, especially polyomaviruses 6 and 7. Most notably, the present disclosure found seven BRCA samples and two HNSC samples that express polyomaviruses. The present disclosure additionally found Parvovirus B19 sequences in a few samples (three cancer and one matched non-cancer); this virus has been mostly associated with normal tissues, but was also previously identified in isolated tumor cases. Additional details and information is found at Cossart, et al., 1975, “Parvovirus-like particles in human sera,” Lancet (1), pg. 72-73; Adamson-Small et al., 2014, “Persistent parvovirus B19 infection in non-erythroid tissues: possible role in the inflammatory and disease process,” Virus Res (190), pg. 8-16; Dickinson et al., 2019, “Newly detected DNA viruses in juvenile nasopharyngeal angiofibroma (JNA) and oral and oropharyngeal squamous cell carcinoma (OSCC/OPSCC),” Eur Arch Otorhinolaryngol (276), pg. 613-617; Li et al., 2007, “Detection of parvovirus B19 nucleic acids and expression of viral VP1/VP2 antigen in human colon carcinoma,” Am J Gastroenterol (102), pg. 1489-1498, each of which is hereby incorporated by reference in its entirety for all purposes. The present disclosure investigated possible genomic correlates of the expression of these viruses, including the tumor mutation burden (TMB, the rate of somatic mutations in a tumor, which is a biomarker and is annotated for all TCGA samples), and the chromosome-level aneuploidy (Methods). The present disclosure determined that HR-αHPV-positive samples have lower TMB and ancuploidy levels compared to HR-αHPV-negative samples (FIG. 6B). In contrast, LIHC cancer patients positive for HBV showed significantly higher TMB compared to HBV-negative samples (FIG. 6B). The present disclosure additionally examined the association between viral expression and overall survival. The present disclosure found that HR-αHPV-positive HNSC patients have significantly better survival compared to HR-αHPV-negative patients (FIG. 6C), in agreement with previous studies. Additional details and information is found at Sethi et al., 2012, “Characteristics and survival of head and neck cancer by HPV status: a cancer registry-based study,” Int J Cancer (131), pg. 1179-1186; Sarkar et al., 2017, “Human papilloma virus (HPV) infection leads to the development of head and neck lesions but offers better prognosis in malignant Indian patients,” Med Microbiol Immunol (206), pg. 267-276, each of which is hereby incorporated by reference in its entirety for all purposes. Notably, the present disclosure found a positive and significant association between viral presence and overall survival of LIHC patients with HBV or AAV2, and of KIRC patients with torqueviruses (FIG. 6C).

### Example 4: Uncovering Expression Patterns of HERVs in Cancer Tissues

To further demonstrate the utility of the disclosed model, the expression of HERVs was analyzed across different tumor types in TCGA (HERVs were not used to train the viRNAtrap model). HERVs constitute approximately 8% of the human genome; most HERV sequences are remnants of ancestral retroviral infection that became fixed in the germline DNA. Additional details and information is found at Curty et al., 2020, “Human Endogenous Retrovirus K in Cancer: A Potential Biomarker and Immunotherapeutic Target,” Viruses (12), doi: 10.3390/v12070726; Kolbe et al., 2020, “Human Endogenous Retrovirus Expression Is Associated with Head and Neck Cancer and Differential Survival,” Viruses (12), doi: 10.3390/v12090956, each of which is hereby incorporated by reference in its entirety for all purposes. While HERV proteins are found expressed in different conditions including cancer tissues, the impact of HERVs on cancer progression and clinical outcomes is not well understood. Additional details and information is found at Kämmerer et al., 2011, “Human endogenous retrovirus K (HERV-K) is expressed in villous and extravillous cytotrophoblast cells of the human placenta,” J Reprod Immunol (91), pg. 1-8; Armbruester et al., 2002, “A novel gene from the human endogenous retrovirus K expressed in transformed cells,” Clin Cancer Res (8), pg. 1800-1807; Wang-Johanning et al., 2008, “Human endogenous retrovirus K triggers an antigen-specific immune response in breast cancer patients,” Cancer Res (68), pg. 5869-5877; Wang-Johanning et al., 2001, “Expression of human endogenous retrovirus k envelope transcripts in human breast cancer,” Clin Cancer Res (7), pg. 1553-1560; Kassiotis, 2014, “Endogenous retroviruses and the development of cancer,” J Immunol (192), pg. 1343-1349, each of which is hereby incorporated by reference in its entirety for all purposes. Specifically, the HERV-K family, which most recently integrated to the human genome and is one of the most abundant HERV families in the human genome (along with HERV-H), was previously reported in tumor tissues and cell lines. Additional details and information is found at Xue et al, 2020, “Human Endogenous Retrovirus K (HML-2) in Health and Disease,” Front Microbiol (11), pg. 1690; Kim et al., 2020, “Crossing the kingdom border: Human diseases caused by plant pathogens,” Environ Microbiol (22), pg. 2485-2495, each of which is hereby incorporated by reference in its entirety for all purposes.

To comprehensively characterize HERV members that are expressed in different tumors, the present disclosure established a database of potentially functional HERVs that were extracted from the human genome (Methods). The model contigs were aligned against this database, to identify patterns of HERV expression in the 14 cancer types considered throughout the present disclosure.

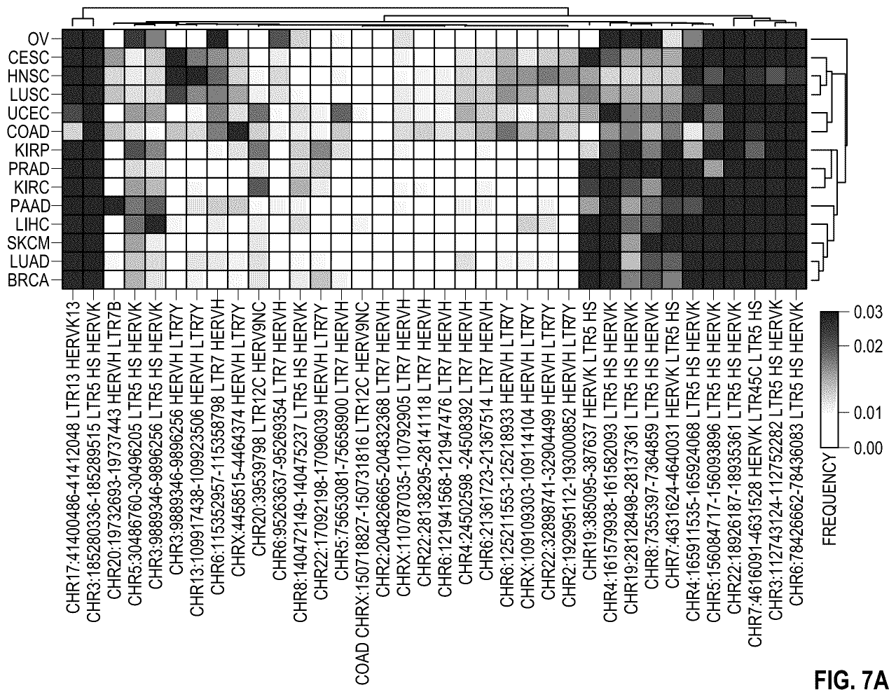

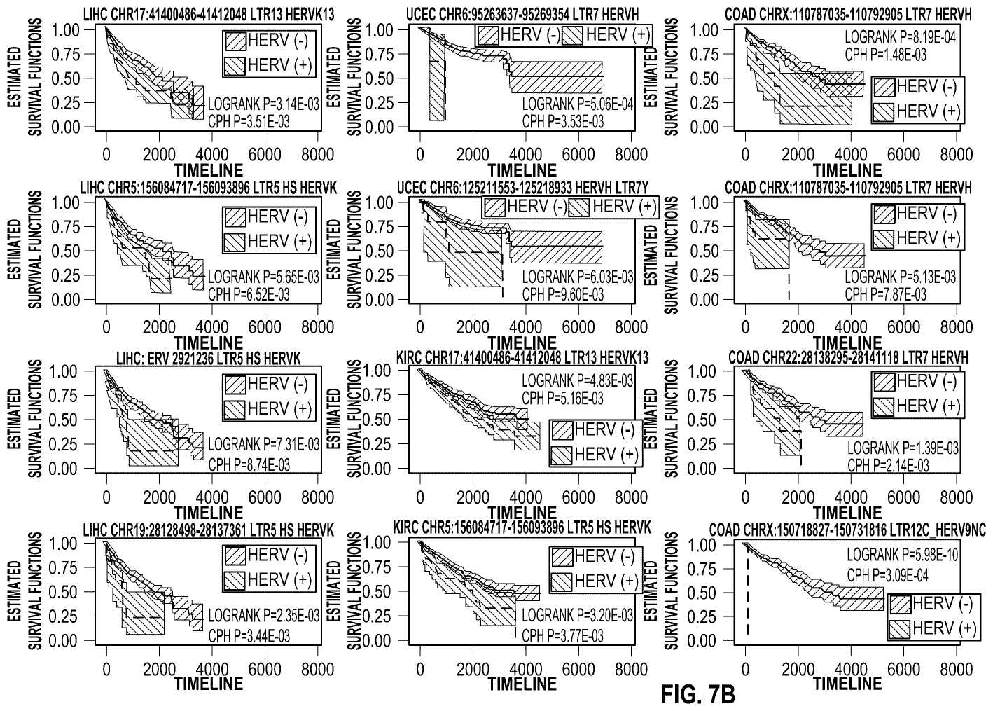

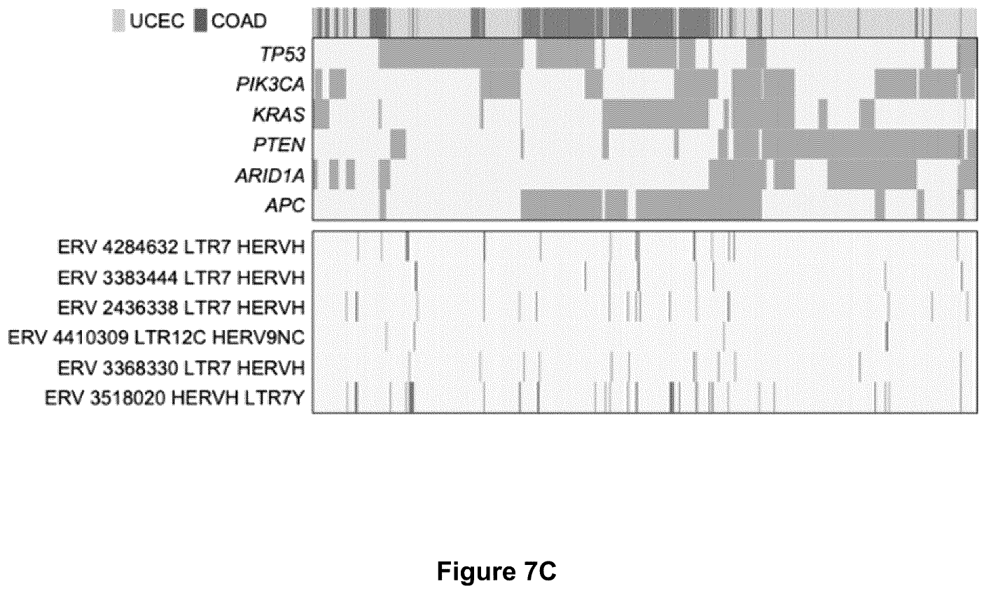

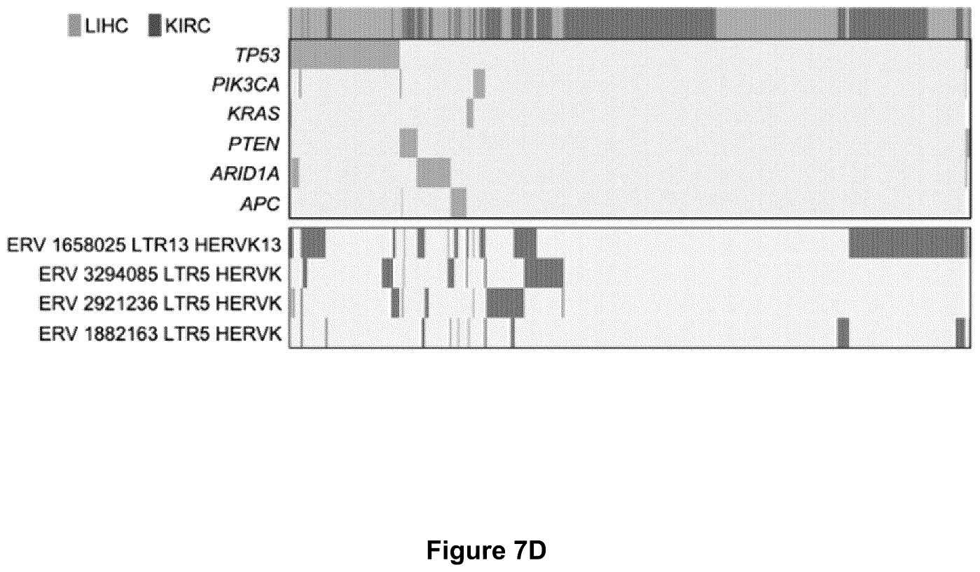

It was determined that the most abundantly expressed HERV families were HERV-K and HERV-H. The fraction of samples expressing different individual HERV members was used to cluster tumor types. Interestingly, it was found that squamous cell carcinomas (including cervical, lung, and head and neck) were clustered together based on the proportional distribution of expressed HERV members (FIG. 7A). The HERVs that were most abundantly expressed across different cancers included some that are in proximity to cancer-associated genes or single nucleotide polymorphisms (SNPs) (Table 4). Specifically, one HERV-H member (chr2:204826665-204832368) was located 365 bp from the ICOS (Inducible T-cell costimulatory) gene, which has been associated with tumor immune responses. Additional details and information is found at Fan et al., 2014, “Engagement of the ICOS pathway markedly enhances efficacy of CTLA-4 blockade in cancer immunotherapy,” J Exp Med (211), pg. 715-725; Xiao et al., 2020, “ICOS Is an Indicator of T-cell-Mediated Response to Cancer Immunotherapy,” Cancer Res (80), pg. 3023-3032; Faget et al., 2012, “ICOS-ligand expression on plasmacytoid dendritic cells supports breast cancer progression by promoting the accumulation of immunosuppressive CD4+ T cells,” Cancer Res (72), pg. 6130-6141; Conrad et al., 2012, “Plasmacytoid dendritic cells promote immunosuppression in ovarian cancer via ICOS costimulation of Foxp3(+) T-regulatory cells,” Cancer Res (72), pg. 5240-5249, each of which is hereby incorporated by reference in its entirety for all purposes. In addition, one HERV9 member (chrX:150718827-150731816) is located 330 bp from the PASDI cancer/testis antigen gene (each of these two HERVs are found in 10 TCGA samples, Table 4).

Associations between HERV transcript presence and patients' overall survival (FIG. 7B) were assessed. It was found that patients with HERV-K- and HERV-H-positive cancer samples had significantly lower overall survival compared to HERV-K- and HERV-H-negative patients in COAD, KIRC, UCEC and LIHC. Notably, every significant association identified between HERV presence and overall survival in these cancer types is negative (Table 5). One HERV-H member (chr22:28138295-28141118) whose expression is significantly associated with poor survival in colon cancer is located 3146 bp from the MNI (meningioma 1) gene, whose high expression has been previously associated with poor survival of colorectal cancer patients. Additional details and information is found at Ho et al., 2019, “High expression of meningioma 1 is correlated with reduced survival rates in colorectal cancer patients,” Acta Histochem (121), pg. 628-637, which is hereby incorporated by reference in its entirety for all purposes.

To investigate the link between HERV expression and poor survival, the TMB and aneuploidy scores were compared between patients expressing HERVs and those without HERV expression. HERVs that were associated with poor survival were not associated with TMB or aneuploidy (Table 6). It was found that HERVs associated with poor overall survival were generally more likely to be expressed in the presence of somatic mutations in frequently mutated cancer driver genes, such as TP53, KRAS, ARIDIA and PTEN (using hyper-geometric enrichment, Table 7). However, a strong association with mutations in any specific gene were not found, and HERV expression was found even in samples with no somatic mutations in any of these genes (FIGS. 7C and 7D, Table 8).

### Example 5: Finding Divergent Viruses in Human Cancer

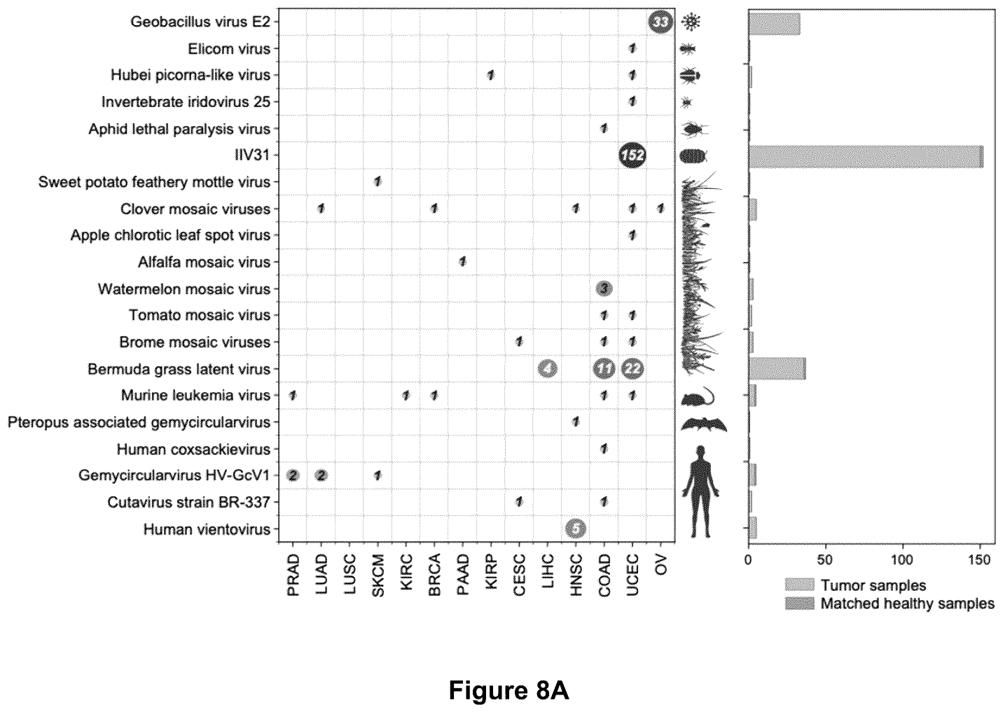

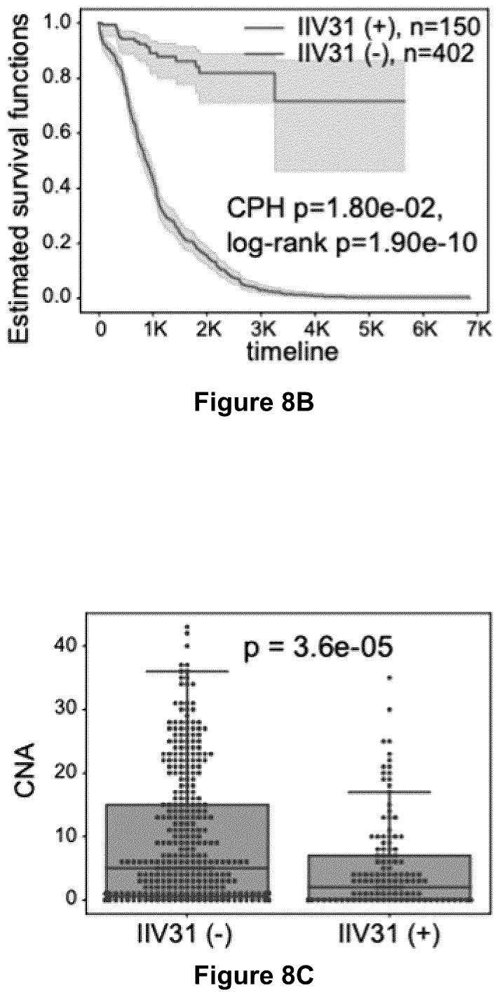

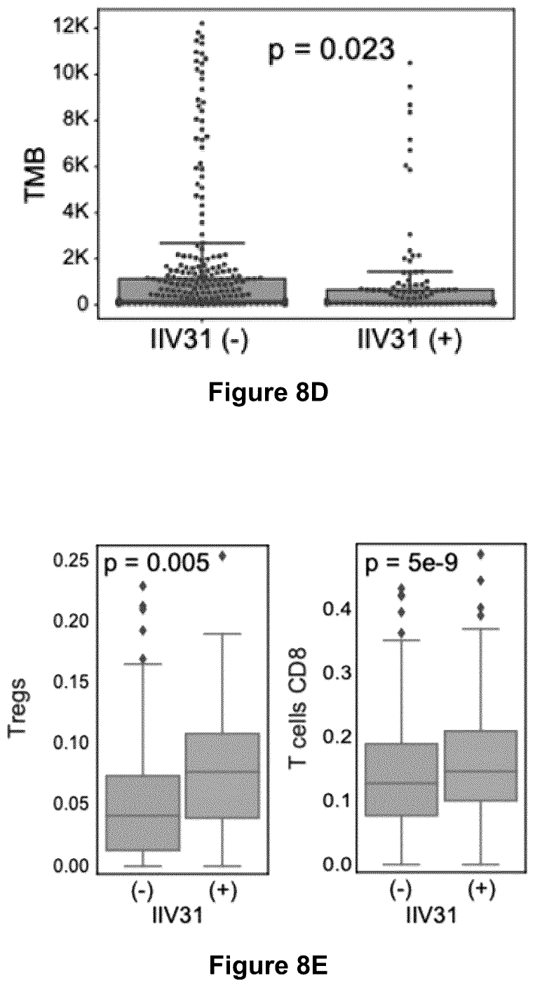

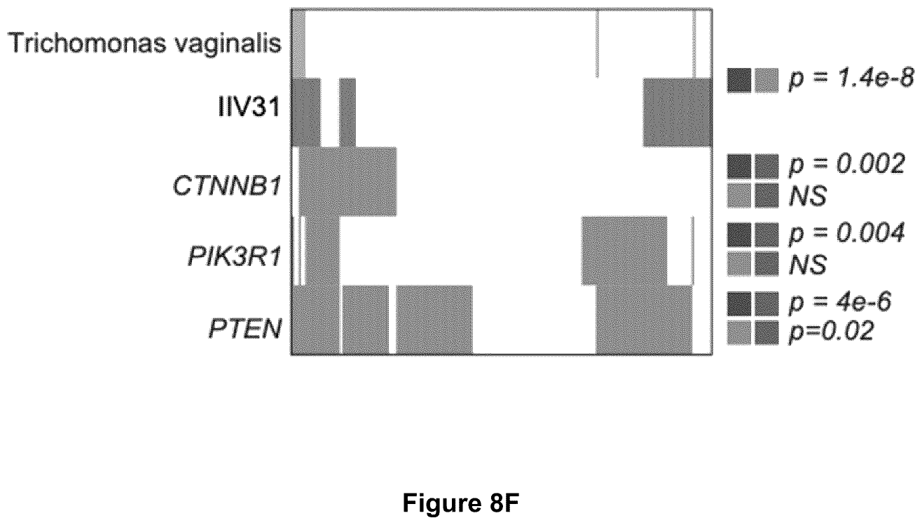

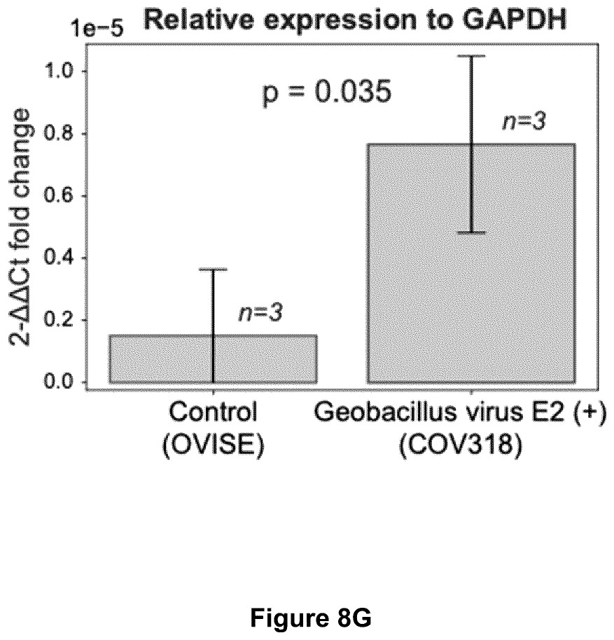

Tumor expression of divergent viruses that have rarely or never been previously reported in human cancers were investigated. Contigs produced by a model in accordance with the present disclosure were aligned against a database of viruses (Methods) from different hosts that were not expected to be found in tumor tissues, including human, bat, mouse, insect, plant, and bacterial viruses. (FIG. 8A). Multiple contigs of mosaic plant viruses were found in distinct samples from most tumor types, especially adenocarcinomas. For example, watermelon mosaic virus was found in 3 colorectal cancer samples, and Bermuda grass latent virus, which was previously reported in a COAD sample, was identified in multiple samples from three cancer types (COAD, LIHC, UCEC; FIG. 8A). Additional details and information is found at Tang et al., 2013, which is hereby incorporated by reference in its entirety for all purposes. Mosaic plant viruses have been previously detected in human feces, which could suggest viral entry and travel through the digestive tract. Additional details and information is found at Zhang et al., 2006m “RNA viral community in human feces: prevalence of plant pathogenic viruses,” PLOS Biol (4), pg. e3, doi: 10.1371/journal.pbio.0040003; Balique et al., 2015, “Can plant viruses cross the kingdom border and be pathogenic to humans?” Viruses (7), pg. 2074-2098, each of which is hereby incorporated by reference in its entirety for all purposes. However, it is unclear how mosaic plant viruses would reach other tumor tissues, such as the liver and the endometrium, and whether these are associated with an unidentified source of laboratory contamination.

Notably, expression in five head and neck carcinoma samples of a Vientovirus were identified, a member of the recently characterized human virus family Redondoviridae that was associated with human oro-respiratory tract (FIG. 8A, Table 9). Additional details and information is found at Abbas et al., 2019, “Redondoviridae, a Family of Small, Circular DNA Viruses of the Human Oro-Respiratory Tract Associated with Periodontitis and Critical Illness,” Cell Host Microbe (25), 719-729.e714, which is hereby incorporated by reference in its entirety for all purposes. Also found was expression of a Gemycircularvirus HV-GcV1 in distinct samples from several cancer types, and Cutavirus expression in one COAD and one CESC sample each. Additional details and information is found at Halary et al., 2016, “Novel Single-Stranded DNA Circular Viruses in Pericardial Fluid of Patient with Recurrent Pericarditis,” Emerg Infect Dis (22), pg. 1839-1841, which is hereby incorporated in its entirety for all purposes. Additionally human coxsackievirus were detected in a COAD sample, confirming a previous report. Additional details and information is found at Dalldorf et al., 1948, “An Unidentified, Filtrable Agent Isolated from the Feces of Children with Paralysis,” Science (108), pg. 61-62; Tang et al., 2013, which is hereby incorporated by reference in its entirety for all purposes.

Expression of a few arthropod viruses were found in TCGA, almost exclusively in UCEC samples (FIG. 8A), most notable of which was Armadillidium vulgare iridescent virus (IIV31). Additional details and information is found at Federici et al., 1980, “Isolation of an iridovirus from two terrestrial isopods, the pill bug, Armadillidium vulgare, and the sow bug, Porcellio dilatatus,” Journal of Invertebrate Pathology (36), pg. 373-381. Reads that align to IIV31 proteins in 152 endometrial cancer samples (which constitute more than 25% of endometrial cancer samples studied) were also detected. While previous reports of IIV31 in these samples were not found, reads that align to the same strain were recently detected in a few DNA sequencing samples, but were filtered because these were not included in databases of multiple pipelines. Additional details and information is found at Zapatka et al., 2020, which is hereby incorporated by reference in its entirety for all purposes. IIV31 is in Betairidovirinae; members of this subfamily of dsDNA viruses infect a wide variety of arthropods, including common insect parasites of humans. Additional details and information is found at Williams, 2008, “Natural invertebrate hosts of iridoviruses (Iridoviridae),” Neotrop Entomol 37, pg. 615-632, which is hereby incorporated by reference in its entirety for all purposes. One study speculated on the role of Betairidovirinae transmitted by mosquitos in human disease, but their presence in humans has not been reported before. Additional details and information is found at Li et al., 2017, “Investigation on Mosquito-Borne Viruses at Lancang River and Nu River Watersheds in Southwestern China,” Vector Borne Zoonotic Dis (17), pg. 804-812, which is hereby incorporated by reference in its entirety for all purposes. While Betairidovirinae are not considered to be pathogens of vertebrates, one study showed that the model Betairidovirinae insect iridovirus 6 (IIV6) was lethal to mice after injection, while heat-inactivated IIV6 was not. Additional details and information is found at Ohba et al., 1982, “Mammalian toxicity of an insect iridovirus,” Acta Virol (26), pg. 165-168, which is hereby incorporated by reference in its entirety for all purposes. Additional studies have shown that Betairidovirinae can infect vertebrate predators of infected insects as well as several vertebrate cell lines. Additional details and information is found at İnce et al., 2018, “Invertebrate Iridoviruses: A Glance over the Last Decade,” Viruses (10), doi: 10.3390/v10040161, which is hereby incorporated by reference in its entirety for all purposes. Therefore, Betairidovirinae may opportunistically infect vertebrates, including humans.

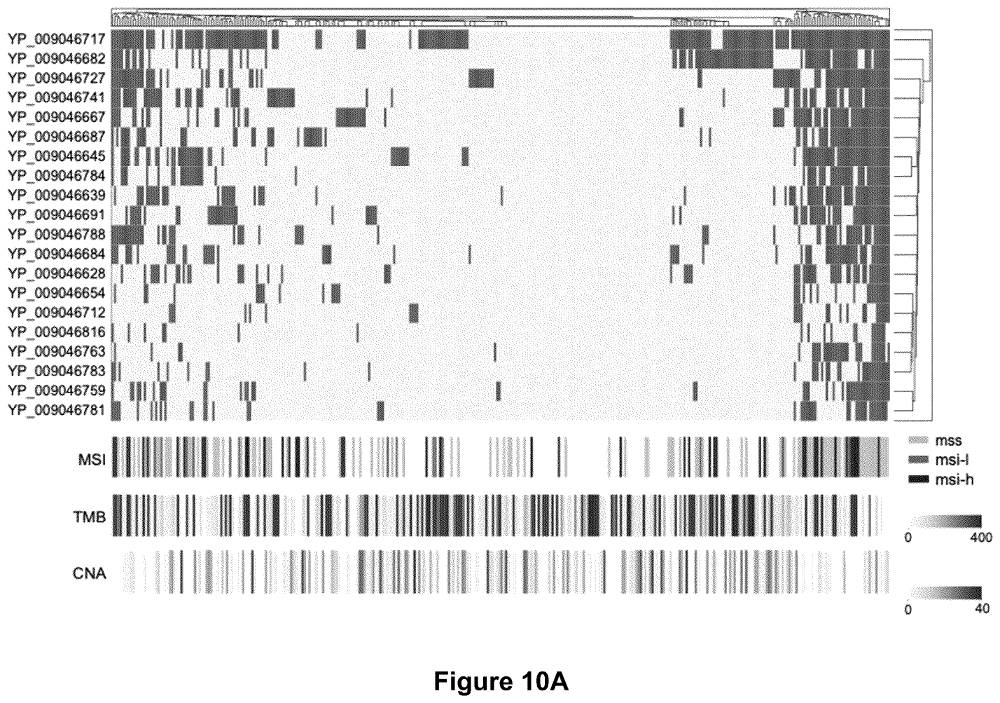

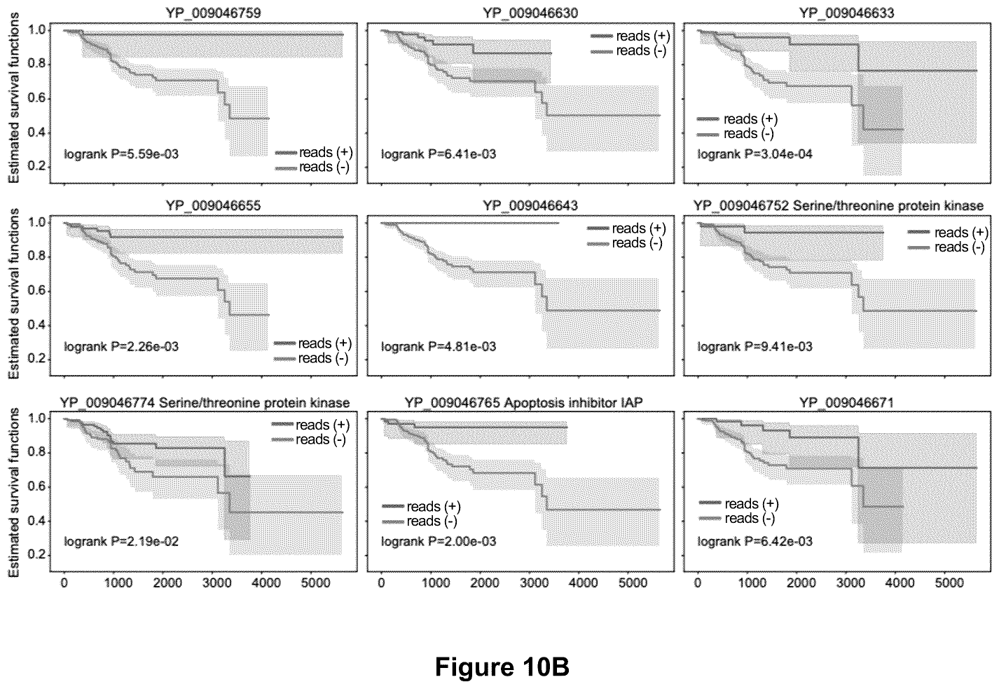

Different IIV31 genes expressed in UCEC samples were identified, and samples positive for IIV31 proteins originate from different batches and sequencing centers (Table 10). In addition, IIV31 presence was found and was strongly and positively associated with overall survival (FIG. 8B), and negatively associated with TMB and chromosome-level ancuploidy (FIGS. 8C and 8D). A path to contamination by IIV31 was not found; the multiple origins of IIV31-positive samples and significant associations between IIV31 expression and other cancer properties both suggest that IIV31 was not a contaminant. Of the most highly expressed IIV31 proteins, an IAP apoptosis inhibitor homolog and serine/threonine protein kinases were found that were individually associated with poor overall survival (YP_009046765, YP_009046752 and YP_009046774, respectively), as well as a RAD50 homolog (YP_009046808, FIGS. 10A and 10B, Table 10).

Significant positive association was found between IIV31 and CIBERSORT inferred CD8T cell frequency and Treg frequency (FIG. 8E). See Newman et al., 2015. These findings, together with the association with improved survival suggest that IIV31 could be linked with a different infection, either directly or indirectly. The association of IIV31 infection with Trichomonas vaginalis (TV) infection was explored. Additional details and information is found at Carlton et al., 2007, “Draft genome sequence of the sexually transmitted pathogen Trichomonas vaginalis,” Science (315), pg. 207-212, which is hereby incorporated by reference in its entirety for all purposes. TV is a single-celled protozoan pathogen that infects the human urogenital tract, and has been associated with increased risk of cervical cancer, which is enhanced by HPV coinfection. Additional details and information is found at Kissinger, 2015, “Trichomonas vaginalis: a review of epidemiologic, clinical and treatment issues” BMC Infect Dis (15), pg. 307; Yang et al., 2018, each of which is hereby incorporated by reference in its entirety for all purposes.

It was determined that TV is expressed in multiple UCEC tumor samples (this was verified 21 TV positive tumors with strict alignment parameters, due to high false positive rate when aligning against TV transcripts). Indeed, TV positive samples are highly enriched with IIV31 positive samples (Fisher exact test p-value=1.4e-8). Both TV and IIV31 are significantly associated with PTEN mutations, which are linked to better survival in endometrial cancers (whereas presence of IIV31 is also associated with mutations in CTNNB1 and PIK3R1, FIG. 8F). Additional details and information is found at Risinger et al., 1998, “PTEN mutation in endometrial cancers is associated with favorable clinical and pathologic characteristics,” Clin Cancer Res (4), pg. 3005-3010, which is hereby incorporated by reference in its entirety for all purposes.

Additionally Geobacillus virus E2 expression was identified in 33 ovarian cancer samples; this virus is likely the most frequently expressed virus in high grade serous ovarian cancer. To further validate the presence of the Geobacillus virus E2, the disclosed model 40 was applied to cell line data from CCLE. Additional details and information is found at Barretina et al., 2012, “The Cancer Cell Line Encyclopedia enables predictive modelling of anticancer drug sensitivity,” Nature (483), pg. 603-607, which is hereby incorporated by reference in its entirety for all purposes. The COV318 cell line was identified as Geobacillus virus E2-positive and the OVISE cell line was identified as a virus-negative control. Through qRT-PCR the expression E2 was validated in the predicted-positive cell line COV318 (FIG. 8G). These results verify that Geobacillus virus E2, which was never found in ovarian cancer before, is indeed expressed in ovarian cancer cells, and that viRNAtrap can be used to sensitively detect virus-positive samples. Geobacillus bacteria has been previously detected multiple ovarian cancer samples. Additional details and information is found at Banerjee et al., “The ovarian cancer oncobiome,” Oncotarget (8), pg. 36225-36245; Nejman et al., 2020, “The human tumor microbiome is composed of tumor type-specific intracellular bacteria,” Science (368), pg. 973-980, each of which is hereby incorporated by reference in its entirety for all purposes. Although the Geobacillus species harboring the phage could not be pinpointed, this was likely within those previously found in ovarian cancer samples. Additional details and information is found at id.

Murine leukemia virus expression was found in distinct samples from five cancer types. Robinson, 1982, “Retroviruses and cancer,” Rev Infect Dis (4), pg. 1015-1025, which is hereby incorporated by reference in its entirety for all purpose. However, murine leukemia virus contamination has been reported for cell culture due to human DNA preparation. Additional details and information is found at Uphoff et al., 2015, “Prevalence and characterization of murine leukemia virus contamination in human cell lines,” PLOS One (10), pg. e0125622, doi: 10.1371/journal.pone.0125622, which is hereby incorporated by reference in its entirety for all purposes.

The present disclosure additionally detected a novel virus in a matched non-cancer sample from one HNSC patient, with protein similarity to Pteropus (fruit bat)-associated Gemycircularvirus and several other gemycircularviruses (Table 9). Cancer patients where IIV31 was found (blue) and patients where IIV31 was not found (red).

### Example 6: Classifying Genetic Events with Distinct Contribution to Tumor Progression and Introduce Ordering of Different Classes of Oncogenic Events

The present disclosure identified representative altered genes for central cancer pathways and explored the temporal dynamics of these alterations at a pathway level. To this end, the core signaling pathways that were altered in cancer were curated. This data was used to distinguish representative altered genes for each pathway. The representative altered genes for each pathway were a minimal group of genes belonging to a biological pathway, such that each tumor sample had genetic alterations in at least one of the genes of the pathway. When such a set of genes was not found, the percentage of samples having an alteration in at least one of the representative genes was alternatively maximized.

For this task, the present disclosure utilized the feature selection framework, and applied the framework for each curated core signaling cancer pathway, to optimize the coverage of tumor samples by selected representative genes of the pathway. Through this approach, many different combinations of genes were evaluated, where the optimal solution with a minimal number of genes was selected for each core signaling cancer pathway. Then, the present disclosure developed temporal ordering models to order of events within each pathway separately, and sort the selected representative genetic alterations of each pathway by an estimate order in which the occur.

Pathways in which dysregulation was crucial for the oncogenic potential of other pathways were distinguished by identifying the pathways that were consistently dysregulated before impairments in other pathways (through a linear or clonal progression). Those consistently early oncogenic pathways present promising therapeutic potential and targeting these pathways may reduce the oncogenic potential of other alterations. The present disclosure searched for components of such pathways that were associated with improved survival and therapeutic responses, which can uncover new drug targets.

### Example 7: Development of an Approach to Identify Complex Combinations of Genetic and Epigenetic Events that Predict Clinical Outcomes

Understanding the molecular and cellular changes that drive cancer initiation and progression is challenging, hindered by the difficulty in the interpretation of noisy omics data. Therefore, complete mutation, copy number variation, methylation, and transcriptome data was processed from thousands of cancer patients (primarily from ICGC/TCGA and MSK-IMPACT pan-cancer cohorts). Additional details and information is found at Campell et al., 2020, “Patterns of somatic structural variation in human cancer genomes,” Nature 578(7793), pg. 112-121; Zehir et al., 2017, “Comprehensive detection of germline variants by MSK-IMPACT, a clinical diagnostic platform for solid tumor molecular oncology and concurrent cancer predisposition testing,” BMC medical genomics 10(1), pg. 1-9, each of which is hereby incorporated by reference in its entirety for all purposes. Notably, an approach was developed that employs Restricted Boltzmann machines (RBMs) to effectively construct prognostic profiles of cancer mutations. In contrast with NMF, RBMs are stochastic and generative neural networks, which are capable of learning a distribution over the input. This allowed the RBM to automatically discover the representations needed for feature detection or classification from raw data, facilitating feature learning and reduction from discrete and sparse data. From this, a new framework was developed that employed machine and deep learning methods to identify the dysregulated processes in cancer that correlate with clinical outcomes.

Accordingly, the present disclosure developed a method to integrate different types of biological datasets to predict treatment responses and clinical outcomes and understand how transcriptional and other types of dysregulations in cancer progression underlie patients' responses. The present disclosure applied this method to publicly available data, to investigate different types of process dysregulation that contribute to response prediction.

### Example 8: Machine Learning Frameworks to Learn Dynamics in Tumor Evolution from Different Data Types and Predict Phenotypic Features and Clinical Outcome

Understanding the mechanisms in tumor evolution underlying response and resistance therapy is critical to improving treatment. The present disclosure therefore developed a framework to study the mutated processes in cancer evolution that determine response to immunotherapy. Through different feature selection and classification methods, the present disclosure has shown that analyzing tumor mutations in the context of biological processes enhances the predictive performance of immunotherapy response compared to existing genomic predictors. Using feature selection methods, subsets of genes were identified within distinct biological processes in which the mutation burden presents an alternative biomarker to the genome-wide tumor mutation burden (TMB). To further enhance the predictive performance, trained nonlinear classifiers using mutated genes in distinct biological processes were used. It was reasoned that nonlinear classification methods have the potential to capture complex associations between ICI responses and mutated genes within a process. It was found that using a random forest method substantially improves the predictive capability of predictors trained using mutations in specific processes, demonstrating significantly better performance compared to the TMB. Among the processes that maintain the best performance are leukocyte and T-cell proliferation regulation, known to play an important role in immune infiltration and ICI treatment. The predictive performance of these process classifiers was consistent across multiple datasets and remained stable across varying sequencing coverage.

Different methods to predict immunotherapy benefit using mutations in the context of biological processes were investigated. This demonstrated several notable improvements over the tumor mutation burden. First, in some embodiments, the models utilize, or require, substantially fewer genes to be sequenced for prediction. Second, developing biomarkers based on distinct biological processes improves their interpretability, and allows investigation of the mechanisms underlining their clinical utility. In particular, it was found that using non-linear classifiers substantially improves the predictive capability of mutated processes, by simultaneously accounting for mutations associated with either resistance or response to treatment. The methods implemented throughout the present disclosure may be applied to construct mutated process predictors of response to other treatments in different cancer types.

Accordingly, the present disclosure investigated mutated biological processes in cancer progression that predicted therapy response by employing different machine learning methods, and pinpoint specific processes that are highly predictive of treatment benefit.

The present disclosure provides a new methodology to construct biologically interpretable genomic predictors for therapy response, by incorporating biological pathway information with machine learning strategies to pinpoint mutated processes that underlie treatment responses.

### Example 9: Identification and Characterizing Unique Viruses and Bacteria that are Associated with Cancer

To allow discovery of new viral-disease associations, the present disclosure provided a deep learning-based framework to rapidly characterize the landscape of viral expression in diverse human tissues. The present disclosure was based on previous establishment of Seeker, a deep learning method and webtool to rapidly identify bacterial viruses from metagenomic sequencing. Additional details and information is found at Auslander et al., 2020, “Seeker: alignment-free identification of bacteriophage genomes by deep learning,” Nucleic Acids Res, 48(21), pg. e121, doi: 10.1093/nar/gkaa856, which is hereby incorporated by reference in its entirety for all purposes. Machine and deep learning techniques can efficiently overcome some of the limitations associated with homology-based approaches and rapidly identify viral reads where seeker has been successfully applied to identify novel and divergent viruses, including new viral families. Additional details and information is found at Auslander et al., 2020; Ren et al., 2017, “VirFinder: a novel k-mer based tool for identifying viral sequences from assembled metagenomic data,” Microbiome, 5(1), pg. 69, each of which is hereby incorporated by reference in its entirety for all purposes.

From this, a model (e.g., model 40) was provided that employs a deep learning model to accurately distinguish viral reads from RNA sequencing, and utilized the model scores to assemble viral contigs. The model was applied to 14 cancer types from TCGA, to characterize the landscape of viral infections in the human cancer transcriptome. The ability of the model to identify different types of viruses that are expressed in tumors by constructing three viral databases was demonstrated. The contigs found by the model were compared to sequence in reference databases. The present disclosure first evaluated known cancer-associated viruses that are expressed in different tumor types. Then, the present disclosure curated a database of potentially functional human endogenous retroviruses (HERVs) and analyzed expression patterns of different HERVs across human cancers to find that HERV expression was associated with poor survival rates. Finally, the present disclosure employed the model to identify divergent viruses that are expressed in tumor tissues. Notably, the present disclosure identified Redondoviridae members that are expressed in head and neck carcinomas, and a Betairdovirinae member that was expressed in more than 25% of endometrial cancer samples. Therefore, the present disclosure provided the first deep learning-based method to identify viruses from human RNA sequencing and demonstrated its ability to rapidly characterize viruses that are expressed in tumors and uncover viral instances that have not been previously found in these samples using alignment based methods. The model was applied to identify new viruses that are expressed in a variety of other malignancies, introducing new avenues to study viral diseases.

Accordingly, to allow identification of novel and divergent viruses in the cancer transcriptome, the present disclosure provided a model, the first computer-implemented technique for alignment free detection of viruses based on deep learning. By combining the scores assigned by the neural network with read assembly, the model allowed identification of divergent viruses that have not been implicated in cancer.

### Example 10: Overview of Exemplary Systems and Methods of the Present Disclosure

The present disclosure provides a model 40, which is an alignment-free method to identify viral reads in RNAseq datasets based on a deep learning model and to assemble predicted viral contigs. Additional details and information is found at Abdurrahamn et al., preprint, “Characterizing the landscape of viral expression in cancer by deep learning,” Github, print, which is hereby incorporated by reference in its entirety for all purposes.

In some embodiments, the systems and methods of the present disclosure includes a plurality of steps, such as four steps.

In some embodiments, the first step included building a Tensorflow model to predict whether reads of a fixed length come from viruses or not. In some embodiments, this was precomputed based on 48 bp reads.

In some embodiments, the second step included, given one or more input files of RNAseq reads (could be paired or unpaired), mapping reads to the human genome. In some embodiments, this was performed independently by a user.

In some embodiments, the third step included using the Tensorflow model to predict which of the unmapped reads are viral. In some embodiments, this was performed by the included systems and methods of the present disclosure.

In some embodiments, the fourth step included assembling the predicted viral reads into longer contigs. In some embodiments, this was performed by using either slow (native Python implementation) or fast (C implementation) modes.

In some embodiments, the systems and methods of the present disclosure utilized an operating system (e.g., operating system 30 of FIG. 1): Linux (CentOS version 7 and Ubuntu 20.04 LTS) and MacOS.

In some embodiments, the systems and methods of the present disclosure utilized python versions: Python 3.6, 3.7, 3.8 and 3.9.

### Example 11: Usage of the Systems and Methods of the Present Disclosure

In some embodiments, to use the systems and methods of the present disclosure, a user provided an input directory including one or more input FASTQ files of unmapped reads with file names ending in *_unmapped.fastq, and a path to an output directory, where a FASTA including predicted viral contigs was generated for each FASTQ of unmapped reads in the input directory. For pairs of files with paired reads from the same sample, which may be stored separately for other sequence analysis, the user was advised to concatenate the two files into one combined file for input to viRNAtrap because viRNAtrap treats each distinct input file as if it comes from a distinct sample.

### Example 12: Exemplary Execution (e.g., Running) of the Systems and Methods of the Present Disclosure

In some embodiments, to execute an instance of the systems and methods of the present disclosure, an example input fastq file is executed.

In some embodiments, the output file generated is evaluated using the expected output.

In some embodiments, there was a one-to-one correspondence between input files in directory input_fastq/and output files in directory output_contigs (or whatever subdirectories the user specifies). If an input file led to zero predicted viral contigs, then the corresponding output file was created but was empty. If one reran the command with the same input files and the same output_contigs/output directory, one first removed the previous output files.

The package came with a small example that was intended to be used to test if one had installed viRNAtrap correctly. The expected output was in subdirectory expected_output. To test if the command worked as expected, run an additional command to compare the output in the new installation to the expected output. The installation was correct if the above diff command returns either no differences or small differences in the less significant digits for the scores in brackets, such as:

5c5
< >contig2[0.8009399]
---
> >contig2[0.8009398]

In some embodiments, the systems and methods of the present disclosure include a fast mode.

The fast mode calls a C library to assemble the viral contigs from the model-predicted viral reads. In some embodiments, the C library was first compiled using a command, such as gcc-o src/assemble_read_c.so-shared-fPIC-O3 src/assemble_read_c.c.

In some embodiments, the library was be compiled using an equivalent command for other C compilers.

In some embodiments, the system and methods of the present disclosure utilized multiple threads, to process multiple input files in parallel, for instance, to operate in parallel using 28 threads.

In some embodiments, in multitreaded mode, the systems and methods of the present disclosure use one thread per file, up to the minimum of the number of available threads and num_threads, where the default num_threads were 48.

In some embodiments, the systems and methods of the present disclosure utilized one or more parameters of Table 11.

TABLE 11
Parameters

| Parameter | Type | Description | Default |
|---|---|---|---|
| input | path (txt) | path to directory | — |
| | | where input fastq is | |
| | | located | |
| output | path (txt) | path to directory | — |
| | | where output fasta | |
| | | will be generated | |
| fastmode | present/absent | run with fast mode | False (no argument) |
| | | (calling C function | |
| | | to assemble viral | |
| | | contigs) | |
| multi_proc | present/absent | run with multi- | False (no argument) |
| | | processing (if | |
| | | multiple files are in | |
| | | the input directory) | |
| num_threads | integer | number of threads to | Integer (no argument or 48 if |
| | | use | multi_proc is True) |
| model_path | path (txt) | path to Tensorflow | /model/model_lr_0.005_pool_5_emb_ |
| | | model to predict | 25_l2_0.02_64.hdf5 |
| | | viral reads | |

In some embodiments, the neural network to predict viral sequences based on 48 bp reads is found in the model directory (model/model_lr_0.005_pool_5_emb_25_12_0.02_64. hdf5). In some embodiments, a user provided trained Tensorflow model replaced this model, but required modification of other functions in virnatrap.

### REFERENCES CITED AND ALTERNATIVE EMBODIMENTS

All references cited herein are incorporated herein by reference in their entirety and for all purposes to the same extent as if each individual publication or patent or patent application was specifically and individually indicated to be incorporated by reference in its entirety for all purposes.

The present invention can be implemented as a computer program product that includes a computer program mechanism embedded in a non-transitory computer-readable storage medium. For instance, the computer program product could contain instructions for operating the user interfaces disclosed herein and described with respect to the Figures. These program modules can be stored on a CD-ROM, DVD, magnetic disk storage product, USB key, or any other non-transitory computer readable data or program storage product.

Many modifications and variations of this invention can be made without departing from its spirit and scope, as will be apparent to those skilled in the art. The specific embodiments described herein are offered by way of example only. The embodiments were chosen and described in order to best explain the principles of the invention and its practical applications, to thereby enable others skilled in the art to best utilize the invention and various embodiments with various modifications as are suited to the particular use contemplated. The invention is to be limited only by the terms of the appended claims, along with the full scope of equivalents to which such claims are entitled.

1. A method for identifying a viral sequence in a subject of a species, the method comprising:
using a computer system comprising one or more processing cores and a memory:
(A) obtaining, in electronic form, a plurality of nucleic acid sequence reads from a biological sample obtained from the subject, wherein the plurality of sequence reads is free of sequence reads associated with a reference genome of the species;
(B) encoding at least a portion of each respective sequence read into a corresponding vector that represents a sequence of the respective sequence read, thereby obtaining a plurality of vectors;
(C) assigning each respective sequence read in the plurality of sequence reads a corresponding scalar model score by inputting the vector, in the plurality of vectors, corresponding to the respective sequence read into a model;
(D) selecting a subset of the plurality of sequence reads as a plurality of contig seeds, wherein each sequence read in the subset of sequence reads has a corresponding scalar model score that satisfies a first threshold score;
(F) aligning sequence reads in the plurality of sequence reads to the plurality of contig seeds through common k-mer sequences thereby forming a plurality of contigs; and
(G) using the plurality of contigs to identify one or more viral sequences in the subject.

2. The method of claim 1, wherein the model includes a one-dimensional convolutional layer followed by one or more fully connected layers.

3. The method of claim 1, wherein the encoding is one hot encoding.

4. The method of claim 1, wherein each sequence read has a length of between 30 base pairs and 400 base pairs.

5. The method of claim 1, wherein the plurality of sequence reads is transcriptomic sequence reads.

6. The method of claim 1, wherein the plurality of sequence reads is genomic sequence reads.

7. The method of claim 1, wherein the aligning (F) to a respective contig seed in the plurality of contig seeds terminates when an average scalar model score for sequence reads aligning to the respective contig seed fails to satisfy a second threshold score.

8. The method of claim 1, wherein each corresponding scalar model score is a value between zero and one and a sequence read in the plurality of sequence reads satisfies the first threshold score when it has a value that is between 0.60 and 1.0.

9. The method of claim 1, wherein each corresponding scalar model score is a value between zero and one and a sequence read in the plurality of sequence reads satisfies the first threshold score when it has a value that is between 0.70 and 1.0.

10. The method of claim 7 wherein each corresponding scalar model score is a value between zero and one and the average scalar model score for sequence reads aligning to the respective seed fails to satisfy the second threshold score when the average scalar model score is less than 0.60.

11. The method of claim 7 wherein each corresponding scalar model score is a value between zero and one and the average scalar model score for sequence reads aligning to the respective seed fails to satisfy the second threshold score when the average scalar model score is less than 0.50.

12. (canceled)

13. The method of claim 1, wherein the common k-mer sequences have a common base pair length, wherein the common base pair length is an integer between 12 and 45.

14. (canceled)

15. The method of claim 2, wherein the model assigns the corresponding model score using an activation function in the final fully connected layer in the one or more fully connected layers.

16. (canceled)

17. (canceled)

18. The method of claim 1, wherein the model comprises 1000 or more weights that are evaluated by the model during the assigning (C) for each respective sequence read in the plurality of sequence reads.

19. The method of claim 1, wherein the model comprises 10,000 or more weights that are evaluated by the model during the assigning (C) for each respective sequence read in the plurality of sequence reads.

20. The method of claim 1, wherein the plurality of sequence reads comprises 10,000 or more sequence reads that each have a length of 35 nucleic acids or more.

21. The method of claim 1, wherein the plurality of sequence reads comprises 100,000 or more sequence reads that each have a length of 35 nucleic acids or more.

22. A computer system for identifying a viral sequence in a subject of a species, the computer system comprising:
at least one processor; and
a memory, the memory storing at least one program for execution by the at least one processor, the at least one program comprising instructions for:
(A) obtaining, in electronic form, a plurality of nucleic acid sequence reads from a biological sample obtained from the subject, wherein the plurality of sequence reads is free of sequence reads associated with a reference genome of the species;
(B) encoding at least a portion of each respective sequence read into a corresponding vector that represents a sequence of the respective sequence read, thereby obtaining a plurality of vectors;
(C) assigning each respective sequence in the plurality of sequence reads a corresponding scalar model score by inputting the vector, in the plurality of vectors, corresponding to the respective sequence read into a model;
(D) selecting a subset of the plurality of sequence reads as a plurality of contig seeds, wherein each sequence read in the subset of sequence reads has a corresponding scalar model score that satisfies a first threshold score;
(F) aligning sequence reads in the plurality of sequence reads to the plurality of contig seeds through common k-mer sequences thereby forming a plurality of contigs; and
(G) using the plurality of contigs to identify one or more viral sequences in the subject.

23. A non-transitory computer-readable storage medium having stored thereon program code instructions that, when executed by a processor, cause the processor to perform a method for identifying a viral sequence in a subject of a species, the method comprising:
(A) obtaining, in electronic form, a plurality of nucleic acid sequence reads from a biological sample obtained from the subject, wherein the plurality of sequence reads is free of sequence reads associated with a reference genome of the species;
(B) encoding at least a portion of each respective sequence read into a corresponding vector that represents a sequence of the respective sequence read, thereby obtaining a plurality of vectors;
(C) assigning each respective sequence read in the plurality of sequence reads a corresponding scalar model score by inputting the vector, in the plurality of vectors, corresponding to the respective sequence read into a model;
(D) selecting a subset of the plurality of sequence reads as a plurality of contig seeds, wherein each sequence read in the subset of sequence reads has a corresponding scalar model score that satisfies a first threshold score;
(F) aligning sequence reads in the plurality of sequence reads to the plurality of contig seeds through common k-mer sequences thereby forming a plurality of contigs; and
(G) using the plurality of contigs to identify one or more viral sequences in the subject.

24. A method for determining a prognosis of a subject surviving a cancer, the method comprising:
using a computer system comprising one or more processing cores and a memory:
(A) obtaining, in electronic form, a plurality of nucleic acid sequence reads from a biological sample obtained from the subject, wherein the plurality of sequence reads is free of sequence reads associated with a reference genome of the species of the subject; and
(B) determining whether sequence reads from an exogenous virus are present in the plurality of sequence reads, wherein,
when sequence reads from an exogenous virus are present in the plurality of sequence reads, the method further comprises up-weighting the prognosis that the subject will survive the cancer, and
when the plurality of sequence reads is free of sequences from an exogenous virus, the method further comprises down-weighting the prognosis that the subject will survive the cancer.

25. The method of claim 24, wherein the exogenous virus is an arthropod virus.

26. The method of claim 25, wherein the arthropod virus is in the Betairidovirinae family.

27. The method of claim 26, wherein the arthropod virus is Armadillidium vulgare iridescent virus.

28. The method of claim 24, wherein the cancer is endometrial cancer.

29. The method of claim 24, wherein the biological sample is a tumor biopsy.

30-31. (canceled)
